(19) United States
(12) Patent Application Publication
(10) Pub. No.: US 2021/0330813 A1
(43) Pub. Date: Oct. 28, 2021

(54) RNAI INDUCED C9ORF72 SUPPRESSION FOR THE TREATMENT OF ALS/FTD

(71) Applicant: Uniqure IP B.V., Amsterdam (NL)

(72) Inventors: Raygene Michaël MARTIER, Amsterdam (NL); Pavlina Stefanova KONSTANTINOVA, Amsterdam (NL)

(21) Appl. No.: 17/196,531

(22) Filed: Mar. 9, 2021

Related U.S. Application Data
(63) Continuation of application No. PCT/EP2019/074198, filed on Sep. 11, 2019.

(30) Foreign Application Priority Data
Sep. 12, 2018 (EP) 18194026.3

(51) Int. Cl. A61K 48/00; C12N 15/86; C12N 15/113

(52) U.S. Cl. CPC A61K 48/005; C12N 15/86; C12N 2310/141; C12N 2750/14143; C12N 2320/32; C12N 15/113

(57) ABSTRACT

The present invention provides for specific target RNA sequences that can in particular be applied in RNAi based gene therapy approaches for the treatment of ALS and/or FTD. These specific target RNA sequences were found by selecting target RNA sequences that were conserved in the C9ORF72 target RNA sequence and were shown to provide for efficient silencing. Also are provided combinations of target RNA sequences that are useful in the treatment of ALS and/or FTD.

### CROSS-REFERENCE TO RELATED APPLICATIONS

This application is a continuation of International Application No. PCT/EP2019/074198 filed Sep. 11, 2019 and claims the benefit of priority to European Patent Application No. 18194026.3 filed Sep. 12, 2018, the entire contents of which are hereby incorporated by reference.

### SEQUENCE LISTING

The instant application contains a Sequence Listing which is being submitted in ASCII format via EFS-WEB and is hereby incorporated by reference in its entirety. Said ASCII copy, created on Mar. 4, 2021, is named 069818-0595SequenceListing.txt and is 44.9 KB.

### BACKGROUND

C9ORF72 is a protein which in humans is encoded by the gene C9ORF72. The human C9ORF72 gene is located on the short (p) arm of chromosome 9 open reading frame 72, from base pair 27,546,542 to base pair 27,573,863. The protein is found in many regions of the brain, in the cytoplasm of neurons as well as in presynaptic terminals. Disease causing mutations, GGGGCC repeats, in the gene were linked to frontotemporal dementia (FTD) and of amyotrophic lateral sclerosis (ALS) and were first reported by DeJesus-Hernandez et al. (Neuron (2011) 72 (2): 245-56) and Renton et al. (Neuron (2011), 72 (2): 257-68.). The presence of the repeats results in the formation of RNA foci (sense, antisense) repeat-associated non-AUG (RAN) peptide translation (sense, antisense), which may underlie the cause of disease.

Hence, as the transcripts of the 5′-GGGGCC-3′ repeats, including corresponding antisense transcripts comprising 5′-GGCCCC-3′, are perceived to be the cause of the disease, approaches have been taken to sequence specifically target C9ORF72 transcripts. The paradigm underlying the approach involves a reduction of mutant protein and/or transcripts to thereby reduce the toxic effects resulting therefrom to achieve a reduction and/or delay of disease symptoms, or even to prevent disease symptoms altogether. Such approaches have focused largely on using synthetic oligonucleotides. Approaches for specific targeting of C9ORF72 transcripts utilising RNAi have also been proposed.

RNA interference (RNAi) is a naturally occurring mechanism that involves sequence specific down regulation of mRNA. The down regulation of mRNA results in a reduction of the amount of protein that is expressed. RNA interference is triggered by double stranded RNA. One of the strands of the double stranded RNA is substantially or completely complementary to its target, the mRNA. This strand is termed the guide strand. The mechanism of RNA interference involves the incorporation of the guide strand in the RNA-induced silencing complex (RISC). This complex is a multiple turnover complex that via complementary base paring binds to its target mRNA. Once bound to its target mRNA it can either cleave the mRNA or reduce translation efficiency. RNA interference has since its discovery been widely used to knock down specific target genes. The triggers for inducing RNA interference that have been employed involve the use of siRNAs or shRNAs. In addition, molecules that can naturally trigger RNAi, the so-called miRNAs, have been used to make artificial miRNAs that mimic their naturally occurring counterparts. These strategies have in common that they provide for substantially double stranded RNA molecules that are designed to target a gene of choice. RNAi based therapeutic approaches that utilise the sequence specific modality of RNAi are under development and several are currently in clinical trials.

### SUMMARY OF THE INVENTION

The present invention provides for specific target RNA sequences that can in particular be applied in RNAi based gene therapy approaches. These specific target RNA sequences were found by selecting target RNA sequences that were conserved in the C9ORF72 target RNA sequence and were shown to provide for efficient silencing of expressed transcripts. Useful target RNA sequences were selected targeting intron 1, exon 2, exon 11 and the antisense transcripts encoded by the human C9ORF72 gene. Useful was also found to be the combination of targeting the antisense transcript and targeting the sense transcripts by selectively targeting a target RNA sequence in intron 1, exon 2, or exon 11.

### DETAILED DESCRIPTION

The current invention relates to gene therapy, and in particular to the use of RNA interference in gene therapy for targeting RNA encoded by the human C9ORF72 gene. Expanded hexanucleotide repeats, (GGGGCCn), in the C9ORF72 gene have been associated with ALS and/or FTD. ALS and/or FTD are diseases of the central nervous system which affect millions of people and for which there is no cure. ALS is characterized by motor neuron degeneration, in the brain and in the spinal cord, that eventually results in respiratory failure, with a median survival of three years after onset. FTD is a common form of early onset dementia affecting younger people. C9ORF72 has been linked to behavioural variant FTD, which involves degeneration of the frontotemperal lobe. The C9ORF72 gene comprising the expanded hexanucleotide repeat, is estimated to be present in about 40% of familial ALS and in about 8-10% of sporadic ALS.

As depicted in FIG. 1, the human C9ORF72 gene expresses several RNA transcripts. The AS RNA transcript, is expressed from within an intronic region and contains the reverse complement of the hexanucleotide repeat (GGCCCC)n. Furthermore, three splice variants of mRNA are expressed, V1, V2 and V3. The V1 transcript does not comprise the repeat sequence. V2 and V3 comprise intronic sequences that contain the hexanucleotide repeats. The hexanucleotide repeats can be translated into polypeptides, being expressed from both sense and antisense transcripts, in so called repeat-associated non-AUG (RAN) translation. Expressed dipeptide repeats (DPR) can be (GA)n, (GR)n, (PA)n, (PR)n and (GP)n). The hexanucleotide repeats from both sense and antisense transcripts also can form RNA foci which can be observed in the nucleus. Hence, reducing RNA expression levels is to reduce DPR and/or RNA foci, which are believed to be associated with disease, thereby benefiting affected patients.

The current invention now provides for an expression cassette encoding a first RNA sequence and a second RNA sequence wherein the first and second RNA sequence are substantially complementary, wherein the first RNA sequence has a sequence length of at least 19 nucleotides and is substantially complementary to a target RNA sequence comprised in an RNA encoded by a human C9ORF72 gene (Reference Sequence: NG_031977.1)).

The first RNA sequence that is to be expressed in according with the invention is to be comprised, in whole or a substantial part thereof, in a guide strand, also referred to as antisense strand as it is complementary (“anti”) to the sense target RNA sequence, the sense target RNA sequence being comprised in an RNA encoded by a human C9ORF72 gene. The second RNA sequence, which is also referred to as “sense strand”, may have substantial sequence identity with or be identical with the target RNA sequence. The first and second RNA sequences are comprised in a double stranded RNA and are substantially complementary. The said double stranded RNA according to the invention is to induce RNA interference to thereby reduce expression of C9ORF72 transcripts, which may include knock down of 5′-GGGGCC-3′ repeat and/or 5′-GGCCCC-3′ repeat containing transcripts, but of non-disease associated C9ORF72 transcripts as well, knocking down both mutant and wild type gene expression. It is understood that substantially complementary in this context means that it is not required to have all the nucleotides of the first and second RNA sequences to be base paired, i.e. to be fully complementary, or all the nucleotides of the first RNA sequence and the target RNA sequence to be base paired. As long as the double stranded RNA is capable of inducing RNA interference to thereby sequence specifically target a sequence comprising the target RNA sequence, such substantial complementarity is contemplated in accordance with the invention.

In one embodiment the double stranded RNA according to the invention comprises a first RNA sequence and a second RNA sequence, wherein the first and second RNA sequence are substantially complementary, and wherein the first RNA sequence has a sequence length of at least 19 nucleotides and is substantially complementary to a target RNA sequence of an RNA encoded by a human C9ORF72 gene, which first RNA sequence is capable of inducing RNA interference to sequence specifically reduce expression of an RNA transcript comprising the target RNA sequence. In a further embodiment, said induction of RNA interference to reduce expression of an RNA transcript comprising the target RNA sequence means that it is to reduce C9ORF72 gene expression.

One can easily determine whether this is the case by using standard luciferase reporter assays and appropriate controls such as described in the examples and as known in the art (Zhuang et al. 2006 Methods Mol Biol. 2006; 342:181-7). For example, a luciferase reporter comprising a target RNA sequence can be used to show that the double stranded RNA according to the invention is capable of sequence specific knock down. Furthermore, such as shown i.a. in the example section, levels of C9ORF72 expression and/or of mutant C9ORF72 can be determined by detecting C9ORF72 RNA (nuclear and/or cytoplasmic), RNA foci, C9ORF72 protein, or C9ORF72 associated dipeptide repeat proteins (DeJesus-Hernandez et al. 2011 Neuron. 72 (2): 245-56) and Ash P E et al. 2013 Neuron. February 20; 77:639.

As said, the double stranded RNA is capable of inducing RNA interference. Double stranded RNA structures that are suitable for inducing RNAi are well known in the art. For example, a small interfering RNA (siRNA) can induce RNAi. An siRNA comprises two separate RNA strands, one strand comprising the first RNA sequence and the other strand comprising the second RNA sequence. An siRNA design that is often used involves 19 consecutive base pairs with a 3′ overhang. The first and/or second RNA sequence may comprise a 3′-overhang. The 3′-overhang preferably is a dinucleotide overhang on both strands of the siRNA. Such a design is based on observed Dicer processing of larger double stranded RNAs that results in siRNAs having these features. The 3′-overhang may be comprised in the first RNA sequence. The 3′-overhang may be in addition to the first RNA sequence. The length of the two strands of which an siRNA is composed may be 19, 20, 21, 22, 23, 24, 25, 26 or 27 nucleotides or more. Each of the two strands comprises the first and second RNA sequence. The strand comprising the first RNA sequence may also consist thereof. The strand comprising the first RNA sequence may also consist of the first RNA sequence and the overhang sequence.

siRNAs may also serve as Dicer substrates. For example, a Dicer substrate may be a 27-mer consisting of two strands of RNA that have 27 consecutive base pairs. The first RNA sequence is positioned at the 3′-end of the 27-mer duplex. At the 3′-ends, like with siRNAs, each or one of the strands may comprise a two nucleotide overhang. The 3′-overhang may be comprised in the first RNA sequence. The 3′-overhang may be in addition to the first RNA sequence. 5′ from the first RNA sequence, additional sequences may be included that are either complementary to the target RNA sequence adjacent or not. The other end of the siRNA dicer substrate is blunt ended. This dicer substrate design may result in a preference in processing by Dicer such that an siRNA can be formed like the siRNA design as described above, having 19 consecutive base pairs and 2 nucleotide overhangs at both 3′-ends. In any case, siRNAs, or the like, are composed of two separate RNA strands (Fire et al. 1998, Nature. 1998 Feb. 19; 391 (6669):806-1 1) each RNA strand comprising or consisting of the first and second RNA sequence in accordance with to the invention.

The first and second nucleotide sequences that are substantially complementary preferably do not form a double stranded RNA of 30 consecutive base pairs or longer, as these can trigger an innate immune response via the double-stranded RNA (dsRNA)-activated protein kinase pathway. Hence, the double stranded RNA is preferably less than 30 consecutive base pairs. Preferably, a pre-miRNA scaffold, a pri-miRNA scaffold, a shRNA, or an siRNA such as designed in accordance with the invention comprising the first and second RNA sequence as described herein does not comprise 30 consecutive base pairs.

The first and second RNA sequences can also be comprised in an shRNA. An shRNA may comprise or consist of from the 5′-end till the 3′-end, 5′-second RNA sequence-loop sequence-first RNA sequence-optional 2 nt overhang sequence-3′. Alternatively, a shRNA may comprise from 5′-first RNA sequence-loop sequence-second RNA sequence—optional 2 nt overhang sequence-3′. Such an RNA molecule forms intramolecular base pairs via the substantially complementary first and second RNA sequence. Suitable loop sequences are well known in the art (i.a. as shown in Dallas et al. 2012 Nucleic Acids Res. 2012 October; 40(18):9255-71 and Schopman et al., Antiviral Res. 2010 May; 86(2):204-11). The loop sequence may also be a stem-loop sequence, whereby the double stranded region of the shRNA is extended. Like the siRNA dicer substrate as described above, a shRNA can be processed by e.g. Dicer to provide for an siRNA having an siRNA design such as described above, having e.g. 19 consecutive base pairs and 2 nucleotide overhangs at both 3′-ends. In case the shRNA is to be processed by Dicer, it is preferred to have the first and second RNA sequence at the end of the shRNA, i.e. such that the putative strands of the siRNA are linked via a stem loop sequence: 5′-first RNA sequence-stem loop sequence-second RNA sequence-optional 2 nt overhang sequence-3′. Or, conversely, 5′-second RNA sequence-stem loop sequence-first RNA sequence-optional 2 nt overhang sequence-3′. Another shRNA design may be a shRNA structure that is processed by the RNAi machinery to provide for an activated RISC complex that does not require Dicer processing (Liu et al., Nucleic Acids Res. 2013, Apr. 1; 41(6):3723-33, incorporated herein by reference), so called AgoshRNAs, which are based on a structure very similar to the miR451 scaffold as described below. Such a shRNA structure comprises in its loop sequence part of the first RNA sequence. Such a shRNA structure may also consist of the first RNA sequence, followed immediately by the second RNA sequence.

A double stranded RNA according to the invention may also be incorporated in a pre-miRNA or pri-mi-RNA scaffold. MicroRNAs, i.e. miRNA, are guide strands that originate from double stranded RNA molecules that are endogenously expressed e.g. in mammalian cells. A miRNA is processed from a pre-miRNA precursor molecule, similar to the processing of a shRNA or an extended siRNA as described above, by the RNAi machinery and incorporated in an activated RNA-induced silencing complex (RISC) (Tijsterman M, Plasterk R H. Dicers at RISC; the mechanism of RNAi. Cell. 2004 Apr. 2; 1 17(1):1-3). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is a shRNA-like molecule that can subsequently be processed by dicer to result in an siRNA-like double stranded RNA duplex. The miRNA, i.e. the guide strand, that is part of the double stranded RNA duplex is subsequently incorporated in RISC. An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31 (13), 3406-3415, 2003), the natural miRNA sequence as it is present in the RNA structure (i.e. duplex, pre-miRNA or pri-miRNA), and the sequence present in the structure that is substantially complementary therewith are removed and replaced with a first RNA sequence and a second RNA sequence according to the invention. The first RNA sequence and the second RNA sequence are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two separate RNA strands that are hybridized via complementary base pairing) as found in nature often are not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first RNA sequence is described e.g. in Liu Y P Nucleic Acids Res. 2008 May; 36(9):281 1-24, which is incorporated herein by reference.

A pri-miRNA can be processed by the RNAi machinery of the cell. The pri-miRNA comprising flanking sequences at the 5′-end and the 3′-end of a pre-miRNA hairpin and/or shRNA like molecule. Such a pri-miRNA hairpin can be processed by Drosha to produce a pre-miRNA. The length of the flanking sequences can vary but may be around 80 nt in length (Zeng and Cullen, J Biol Chem. 2005 Jul. 29; 280(30):27595-603; Cullen, Mol Cell. 2004 Dec. 22; 16(6):861-5). The minimal length of the single-stranded flanks can easily be determined as when it becomes too short, the RNA molecule may lose its function because e.g. Drosha processing fails resulting in sequence specific inhibition being reduced or even absent. In one embodiment, the pri-miRNA scaffold carrying the first and second RNA sequence according to the invention has a 5′-sequence flank and a 3′ sequence flank relative to the predicted pre-miRNA structure of at least 5, at least 10, at least 15, at least 20, at least 30, at least 40, or at least 50 nucleotides. Preferably, the pri-miRNA derived flanking sequences (5′ and 3′) comprised in the miRNA scaffold are derived from the same naturally occurring pri-miRNA sequence. Preferably, pre-miRNA and/or the pri-miRNA derived flanking sequences (5′ and 3′) and/or loop sequences comprised in the miRNA scaffold are derived from the same naturally occurring pri-miRNA sequence, e.g. as shown and listed in table 16. As the (putative) guide strand RNA as comprised in the endogenous miRNA sequence can be replaced by a sequence including (or consisting of) the first RNA sequence, and the passenger strand sequence replaced by a sequence including (or consisting of) the second RNA sequence, it is understood that flanking sequences and/or loop sequences of the pri-miRNA or pre-miRNA sequences of the endogenous sequence may include minor sequence modifications such that the predicted structure of the scaffold miRNA sequence (e.g. M-fold predicted structure) is the same as the predicted structure of the endogenous miRNA sequence.

The first and second RNA sequence, which can form a double stranded RNA, of the invention are encoded by an expression cassette. It is understood that when the double stranded RNA is to be e.g. an siRNA, consisting of two RNA strands, that there may be two expression cassettes required. One encoding an RNA strand comprising the first RNA sequence, the other cassette encoding an RNA strand comprising the first RNA strand. When the double stranded RNA is comprised in a single RNA molecule, e.g. encoding a shRNA, pre-miRNA or pri-miRNA, one expression cassette may suffice. A pol II expression cassette may comprise a promoter sequence a sequence encoding the RNA to be expressed followed by a polyadenylation sequence. In case the double stranded RNA that is expressed comprises a pri-miRNA scaffold, the encoded RNA sequence may encode for intron sequences and exon sequences and 3′-UTR's and 3′-UTRs. A pol III expression cassette in general comprises a promoter sequence, followed by a sequence encoding the RNA (e.g. shRNA sequence, pre-miRNA, or a strand of the double stranded RNAs to be comprised in e.g. an siRNA or extended siRNA). A pol I expression cassette may comprise a pol I promoter, followed by the RNA encoding sequence and a 3′-Box. Expression cassettes for double stranded RNAs are well known in the art, and any type of expression cassette can suffice, e.g. one may use a pol III promoter, a pol II promoter or a pol I promoter (i.a. ter Brake et al., Mol Ther. 2008 March; 16(3):557-64, Maczuga et al., BMC Biotechnol. 2012 Jul. 24; 12:42).

As is clear from the above, the first and second RNA sequence that are comprised in a double stranded RNA can contain additional nucleotides and/or nucleotide sequences. The double stranded RNA may be comprised in a single RNA sequence or comprised in two separate RNA strands. Whatever design is used, it is designed such that from the first and second RNA sequence an antisense RNA molecule comprising the first RNA sequence, in whole or a substantial part thereof, of the invention can be processed by the RNAi machinery such that it is incorporated in the RISC complex to have its action, i.e. to induce RNAi against the RNA target sequence comprised in an RNA encoded by the C9ORF72 gene. The sequence comprising or consisting of the first RNA sequence, in whole or a substantial part thereof, being capable of sequence specifically targeting RNA encoded by a human C9ORF72 gene. Hence, as long as the double stranded RNA is capable of inducing RNAi, such a double stranded RNA is contemplated in the invention. In one embodiment, the double stranded RNA according to the invention is comprised in a pre-miRNA scaffold, a pri-miRNA scaffold, a shRNA, or an siRNA. Preferably the first and second RNA sequence as encoded by the expressed cassette are to be contained in a single transcript. It is understood that the expressed transcript in subsequent processing, i.e. cleavage, results in the single transcript being processed into multiple separate RNA molecules.

The term complementary is herein defined herein as nucleotides of a nucleic acid sequence that can bind to another nucleic acid sequence through hydrogen bonds, i.e. nucleotides that are capable of base pairing. Ribonucleotides, the building blocks of RNA are composed of monomers (nucleotides) containing a sugar, phosphate and a base that is either a purine (guanine, adenine) or pyrimidine (uracil, cytosine). Complementary RNA strands form double stranded RNA. A double stranded RNA may be formed from two separate complementary RNA strands or the two complementary RNA strands may be comprised in one RNA strand. In complementary RNA strands, the nucleotides cytosine and guanine (C and G) can form a base pair, guanine and uracil (G and U), and uracil and adenine (U and A) can form a base pair as well. The term substantial complementarity means that is not required to have the first and second RNA sequence to be fully complementary, or to have the first RNA sequence and target RNA sequence or sequences of RNA encoded by a human C9ORF72 gene to be fully complementary.

The substantial complementarity between the first RNA sequence and the target RNA sequence preferably consists of at most two mismatched nucleotides, more preferably having one mismatched nucleotide, most preferably having no mismatches. It is understood that one mismatched nucleotide means that over the entire length of the first RNA sequence when base paired with the target RNA sequence one nucleotide does not base pair with the target RNA sequence. Having no mismatches means that all nucleotides of the first RNA sequence base pair with the target RNA sequence, having 2 mismatches means two nucleotides of the first RNA sequence do not base pair with the target RNA sequence. The first RNA sequence may also comprise additional nucleotides that do not have complementarity to the target RNA sequence, and may be longer than e.g. 21 nucleotides, in such a scenario, the substantial complementarity is determined over the entire length of the target RNA sequence. This means that the target RNA sequence in this embodiment has either no, one or two mismatches over its entire length when base paired with the first RNA sequence.

As shown in the example section, double stranded RNAs comprising a first nucleotide sequence length of 21 and 22 nucleotides were tested. These first RNA sequences had no mismatches and were fully complementary with the target RNA sequence. Having a few mismatches between the first nucleotide sequence and the target RNA sequence may however be allowed according to the invention, as long as the double stranded RNA according to the invention is capable of reducing expression of transcripts comprising the target RNA sequence, such as a luciferase reporter or e.g. a transcript comprising the target RNA sequence. In this embodiment, substantial complementarity between the first RNA sequence and the target RNA sequence consists of having no, one or two mismatches over the entire length of either the first RNA sequence or the target RNA sequence encoded by an RNA of the human C90RF72, whichever is the shortest.

As said, a mismatch according to the invention means that a nucleotide of the first RNA sequence does not base pair with the target RNA sequence encoded by an RNA of the human C90RF72. Nucleotides that do not base pair are A and A, G and G, C and C, U and U, A and C, C and U, or A and G. A mismatch may also result from a deletion of a nucleotide, or an insertion of a nucleotide. When the mismatch is a deletion in the first RNA sequence, this means that a nucleotide of the target RNA sequence is not base paired with the first RNA sequence when compared with the entire length of the first RNA sequence. Nucleotides that can base pair are A-U, G-C and G-U. A G-U base pair is also referred to as a G-U wobble, or wobble base pair. In one embodiment the number of G-U base pairs between the first RNA sequence and the target RNA sequence is 0, 1 or 2. In one embodiment, there are no mismatches between the first RNA sequence and the target RNA sequence and a G-U base pair or G-U pairs are allowed. Preferably, there may be no G-U base pairs between the first RNA sequence and the target RNA sequence, or the first RNA sequence and the target RNA sequence only have base pairs that are A-U or G-C. Preferably, there are no G-U base pairs and no mismatches between the first RNA sequence and the target RNA sequence. The first RNA sequence of the double stranded RNA according to invention preferably is fully complementary to the target RNA sequence, said complementarity consisting of G-U, G-C and A-U base pairs. The first RNA sequence of the double stranded RNA according to invention more preferably is fully complementary to the target RNA sequence, said complementarity consisting of G-C and A-U base pairs.

In one embodiment the first RNA sequence and the target RNA sequence have at least 15, 16, 17, 18, or 19 nucleotides that base pair. Preferably the first RNA sequence and the target RNA sequence are substantially complementary, said complementarity comprising at least 19 base pairs. In another embodiment, the first RNA sequence has at least 8, 9, 10, 11, 12, 13 or 14 consecutive nucleotides that base pair with consecutive nucleotides of the target RNA sequence. In another embodiment, the first RNA sequence has at least 19 consecutive nucleotides that base pair with consecutive nucleotides of the target RNA sequence. In another embodiment the first RNA sequence comprises at least 19 consecutive nucleotides that base pair with 19 consecutive nucleotides of the target RNA sequence. In still another embodiment, the first RNA sequence has at least 17 nucleotides that base pair with the target RNA sequence and has at least 15 consecutive nucleotides that base pair with consecutive nucleotides of the target RNA sequence. The sequence length of the first nucleotide is preferably at most 21, 22, 23, 24, 25, 26, or 27 nucleotides. In another embodiment, the first RNA sequence has at least 20 consecutive nucleotides that base pair with 20 consecutive nucleotides of the target RNA sequence. In another embodiment the first RNA sequence comprises at least 21 consecutive nucleotides that base pair with 21 consecutive nucleotides of the target RNA sequence.

As said, it may be not required to have full complementarity (i.e. full base pairing (no mismatches) and no G-U base pairs) between the first nucleotide sequence and the target RNA sequence as such a first nucleotide sequence can still allow for sufficient suppression of gene expression. Also, not having full complementarity may be contemplated for example to avoid or reduce off-target RNA sequence specific gene suppression while maintaining sequence specific inhibition of transcripts comprising the target RNA sequence. However, it may be preferred to have full complementarity as it may result in more potent inhibition. Without being bound by theory, having full complementarity between the first RNA sequence and the target RNA sequence may allow for the activated RISC complex comprising said first RNA sequence (or a substantial part thereof) to cleave its target RNA sequence, whereas having mismatches may hamper cleavage and can result in mainly allowing inhibition of translation, of which the latter may result in less potent inhibition.

With regard to the second RNA sequence, the second RNA sequence is substantially complementary with the first RNA sequence. The second RNA sequence combined with the first RNA sequence forms a double stranded RNA. As said, this is to form a suitable substrate for the RNA interference machinery such that a guide sequence derived from the first RNA sequence is comprised in the RISC complex in order to sequence specifically inhibit expression of its target, i.e. RNA encoded by a human C9ORF72 gene. The sequence of the second RNA sequence has sequence similarity with the target RNA sequence. However, the substantial complementarity if the second RNA sequence with the first RNA sequence may be selected to have less substantial complementarity as compared with the substantial complementarity between the first RNA sequence and the target RNA sequence. Hence, the second RNA sequence may comprise 0, 1, 2, 3, 4, or more mismatches, 0, 1, 2, 3, or more G-U wobble base pairs, and may comprise insertions of 0, 1, 2, 3, 4, nucleotides and/or deletions of 0, 1, 2, 3, 4, nucleotides. Preferably the first RNA sequence and the second RNA sequence are substantially complementary, said complementarity comprising 0, 1, 2 or 3 G-U base pairs and/or wherein said complementarity comprises at least 17 base pairs. These mismatches, G-U wobble base pairs, insertions and deletions, are with regard to the first RNA sequence, i.e. the double stranded region that is formed between the first and second RNA sequence. As long as the first and second RNA sequence can substantially base pair, and are capable of inducing sequence specific inhibition of an RNA encoded by a human C9ORF72 gene, such substantial complementarity is allowed according to the invention. It is also understood that substantially complementarity between the first RNA sequence and the second RNA sequence may depend on the double stranded RNA design of choice. It may depend for example on the miRNA scaffold that is chosen for in which the double stranded RNA is to be incorporated.

As is clear from the above, the substantial complementarity between the first RNA sequence and the second RNA sequence, may comprise mismatches, deletions and/or insertions relative to a first and second RNA sequence being fully complementary (i.e. fully base paired). In one embodiment, the first and second RNA sequences have at least 11 consecutive base pairs. Hence, at least 11 consecutive nucleotides of the first RNA sequence and at least 11 consecutive nucleotides of the second RNA sequence are fully complementary. In another embodiment the first and second RNA sequence have at least 15 nucleotides that base pair. Said base pairing between at least 15 nucleotides of the first RNA sequence and at least 15 nucleotides of the second RNA sequence may consist of G-U, G-C and A-U base pairs, or may consist of G-C and A-U base pairs. In another embodiment, the first and second RNA sequence have at least 15 nucleotides that base pair and have at least 11 consecutive base pairs. In another embodiment, the first RNA sequence and the second RNA sequence are substantially complementary, wherein said complementarity comprises at least 17 base pairs. Said 17 base pairs may preferably be 17 consecutive base pairs, said base pairing consisting of G-U, G-C and A-U base pairs or consisting of G-C and A-U base pairs.

As said, the current invention now provides for an expression cassette encoding a first RNA sequence and a second RNA sequence wherein the first and second RNA sequence are substantially complementary, wherein the first RNA sequence has a sequence length of at least 19 nucleotides and is substantially complementary to a target RNA sequence comprised in an RNA encoded by a human C9ORF72 gene. As shown in the examples, suitable target RNA sequences in accordance with the invention are provided (see e.g. table 14). Hence, in one embodiment, an expression cassette is provided encoding a first RNA sequence and a second RNA sequence wherein the first and second RNA sequence are substantially complementary, wherein the first RNA sequence has a sequence length of at least 19 nucleotides and is substantially complementary to a target RNA sequence selected from the group listed in table 14 comprised in an RNA encoded by a human C9ORF72 gene.

Preferably, the target RNA sequence in accordance with the invention is selected from the group listed in table 1 below, consisting of SEQ ID NOs. 2, 4, 15, 21, 31, 32, and 46.

TABLE 1
Selected target RNA sequences in C9ORF72.

| | SEQ ID | TARGET RNA SEQUENCE | |
|---|---|---|---|
| ref. | NO. | (5′-NNNN-3′) | length |
| C2 | 2 | UCACAGUACUCGCUGAGGGUGA | 22 |
| C4 | 4 | AACUCAGGAGUCGCGCGCUAGG | 22 |
| C15 | 15 | UUCCCGGCAGCCGAACCCCAAA | 22 |
| C21 | 21 | GCAGGCAAUUCCACCAGUCGCU | 22 |
| C31 | 31 | GCCCAAGAGUUUGAAGUUACC | 21 |
| C32 | 32 | AUUCUUGGUCCUAGAGUAAGGC | 22 |
| C46 | 46 | UCUCUUCGGAACCUGAAGAUAG | 22 |

Table 1. SEQ ID NOs. 2, 4 and 31 correspond with target RNA sequences of transcripts encoded by the human C9ORF72 gene of intron 1; SEQ ID NO. 15 and 21 corresponds with a target RNA sequence in antisense transcripts encoded by the human C9ORF72 gene; SEQ ID NO. 32 corresponds with a target RNA sequence of transcripts encoded by the human C9ORF72 gene of exon 2; SEQ ID NO. 46 corresponds with a target RNA sequence of transcripts encoded by the human C9ORF72 gene of exon 11.

For these target RNA sequences it was found that surprisingly highly advantageous suitable first and second RNA sequences could be made in accordance with the invention to provide for an expression cassette encoding said first RNA sequence and said second RNA sequence, wherein the first and second RNA sequence are substantially complementary, wherein the first RNA sequence has a sequence length of at least 19 nucleotides and is substantially complementary to one of said target RNA sequences to highly efficiently induce RNAi to reduce C9ORF72 gene expression. It is understood that the reduction of gene expression may include a reduction of transcripts that encode hexanucleotide repeat sequences as well.

As shown in the examples, the first and second RNA sequence of the invention, may be preferably incorporated in a pre-miRNA or a pri-miRNA scaffold derived from miR101 or a pri-miRNA or pre-miRNA scaffold derived from miR451. Pri-miRNA scaffolds for miR451 and miR101 are depicted in FIG. 4B. These scaffolds were found to be in particular useful as these scaffolds both can induce RNA interference and can be combined in a single transcript. These scaffolds also allow to induce RNA interference that can result in mainly guide strand induced RNA interference. The pri-miR451 scaffold does not result in a passenger strand because the processing is different from the canonical miRNA processing pathway (Cheloufi et al., 2010 Jun. 3; 465(7298):584-9 and Yang et al., Proc Natl Acad Sci USA. 2010 Aug. 24; 107(34):15163-8). The pri-miR101 scaffold is produced by the canonical miRNA processing pathway but it was found that many of the miR101 scaffolds produced mainly guide strands (see e.g. C2, C4, C32, and C33) and very low amounts of passenger strands. Hence, both scaffolds represent excellent candidates to develop a gene therapy product as unwanted potential off-targeting by passenger strands can be largely, if not completely, avoided. As the passenger strand (corresponding to the second sequence) may result in targeting of transcripts other than C9ORF72 RNA, using such scaffolds may allow one to avoid such unwanted targeting. Hence, it is preferred that scaffolds are selected that produce less than 5% of passenger strands, more preferably less than 4%, most preferably less than 3% of passenger strands.

As is shown in the examples, a first RNA sequence of 21 (for a miR101 scaffold) or 22 nucleotides (e.g. for a miR451) in length may be selected and incorporated in a miRNA scaffold. Such a miRNA scaffold sequence is subsequently processed by the RNAi machinery as present in the cell. When reference is made to miRNA scaffold it is understood to comprise pri-miRNA structures or pre-miRNA structures. As shown in the examples, such miRNA scaffolds, when processed in a cell, result in guide sequences comprising the first RNA sequence, or a substantial part thereof, in the range of 18-23 nucleotides in length for the 101 scaffold and in the range of 21-30 nucleotides in length for the 451 scaffold. Such guide strands being capable of reducing C9ORF72 transcript expression by targeting the selected target sequences. As is clear from the above, and as shown in the examples, the first RNA sequence as it is encoded by the expression cassette of the invention, is comprised in part or in whole, in a guide strand when it has been processed by the RNAi machinery of the cell. Hence, the guide strand that is to be generated from the RNA encoded by the expression cassette, comprising the first RNA sequence and the second RNA sequence is to comprise at least 18 nucleotides of the first RNA sequence. Preferably, such a guide strand comprises at least 19 nucleotides, 20 nucleotides, 21 nucleotides, or 22 nucleotides. A guide strand can comprise the first RNA sequence also as a whole. In selecting a miRNA scaffold, the first RNA sequence can be selected such that it is to replace the original guide strand. As shown in the example section, this does not necessarily mean that guide strand produced from such an artificial scaffold are identical in length as the first RNA sequence selected, nor that the first RNA sequence is in its entirety to be found in the guide strand that is produced.

A miRNA 451 scaffold, as shown in the examples, and as shown in FIG. 4b and FIG. 34 preferably comprises from 5′ to 3′, firstly 5′-CUUGGGAAUGGCAAGG-3′ (SEQ ID NO. 112), followed by a sequence of 22 nucleotides, comprising or consisting of the first RNA sequence, followed a sequence of 17 nucleotides, which can be regarded to be the second RNA sequence, which is complementary over its entire length with nucleotides 2-18 of said sequence of 22 nucleotides, subsequently followed by sequence 5′-MWCUUGCUAUCCCAGA-3′ (wherein M is an A or a C and W is an A or a U) (SEQ ID NO.113). Preferably the first 5′-C nucleotide of the latter sequence is not to base pair with the first nucleotide of the first RNA sequence. Such a scaffold may comprise further flanking sequences as found in the original pri-miR451 scaffold. Alternatively, the flanking sequences, 5′-CUUGGGAAUGGCAAGG′-3′ and 5′-MWCUUGCUAUCCCAGA-3′ may be replaced by flanking sequences of other pri-mRNA structures. As is clear from the above, the sequence of the scaffold may differ not only with regard to the (putative) guide strand sequence, and sequence complementary thereto, as present in the wild-type scaffold (FIG. 4b), but may also comprise additional mutations in the 5′, loop and 3′ sequence as well (see FIG. 34), as additional mutations may be required to provide for an RNA structure that is predicted to mimic the secondary structure of the wild-type scaffold. Such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention thus expressing a shRNA-like structure having a sequence of 22 nucleotides, comprising or consisting of the first RNA sequence, followed a sequence of 17 nucleotides, which can be regarded to be the second RNA sequence, which is complementary over its entire length with nucleotides 2-18 of said sequence of 22 nucleotides. The latter shRNA-like structure derived from the miR451 scaffold can be referred to as a pre-miRNA scaffold from miR451.

A miRNA 101 scaffold, as shown in the examples and in FIGS. 4b and 33, preferably comprises from 5′ to 3′, firstly 5′-UGCCCKGGNN-3′ (SEQ ID NO.114; wherein N can be any nucleotide; K is C or U), followed by a second RNA sequence of 22 nucleotides in length, subsequently followed by a loop sequence 5′ SUCUAUUCUAAANN-′3 (SEQ ID NO.115), (S is G or C), wherein the last 3′ two nucleotides of the loop sequence are to base pair with the last 3′ nucleotides of the second RNA sequence, followed by a first RNA sequence of 21 nucleotides in length, wherein the first 20 consecutive nucleotides are complementary (i.e. base pair) to the second RNA sequence and the last N of said 5′-UGCCCKGGNN-3′ sequence, and wherein the last 3′ nucleotide does not form a base-pair with said 5′-UGCCCKGGNN-3′ sequence. The second RNA sequence comprises a bulge (non-base paired nucleotide) at position 5, counting from the 3′-end, of the said 22 nucleotides. Lastly, the miRNA 101 scaffold comprises the sequence 5′-RSAUGGCA-3′ (SEQ ID NO.116; wherein R is an A, U or a G).As is clear from the above, the sequence of the scaffold differs not only with regard to the (putative) guide and passenger strand sequences as present in the wild-type scaffold (FIG. 4b), but may also comprise additional mutations in the 5′, loop and 3′ sequence as well (see FIG. 33), as additional mutations may be required to provide for an RNA structure that is predicted to mimic the secondary structure of the wild-type scaffold. Such a scaffold may be comprised in a larger RNA transcript, e.g. a pol II expressed transcript, comprising e.g. a 5′ UTR and a 3′UTR and a poly A. Such a scaffold may comprise further flanking sequences as found in the original pri-mi101 scaffold. Alternatively, the flanking sequences, 5′-UGCCCKGGNN-3′ and 5′-RSAUGGCA-3′ may be replaced by flanking sequences of other pri-mRNA structures. Flanking structures may also be absent. An expression cassette in accordance with the invention thus expressing a shRNA-like structure, a pre-miRNA structure of miR101. Such a shRNA-like structure consisting of, starting at the 5′-end, a second RNA sequence of 22 nucleotides in length, subsequently followed by a loop sequence SUCUAUUCUAAANN-′3 (SEQ ID NO.115), wherein the last 3′ two nucleotides of the loop sequence are to base pair with the last 3′ nucleotides of the second RNA sequence, followed by a first RNA sequence of 21 nucleotides in length, wherein the first 20 consecutive nucleotide are complementary to the second RNA sequence. The second RNA sequence comprises a bulge (non-base paired nucleotide) at position 5, counting from the 3′-end, of the said 22 nucleotides.

In one embodiment, an expression cassette according to the invention is provided, wherein said first RNA sequence is substantially complementary to a target RNA sequence comprised in antisense RNA transcripts encoded by the human C9ORF72 gene. Preferably said first RNA sequence is substantially complementary to SEQ ID NO. 15 or 21. More preferably said first RNA sequence has a length of 19, 20, 21, or 22 nucleotides. More preferably said first RNA sequence is fully complementary over its entire length with said first RNA target sequence. Most preferably said first RNA sequence has a length of 19, 20, 21, or 22 nucleotides, wherein said first RNA sequence is fully complementary over its entire length with said first RNA target sequence. Said first RNA strand can be SEQ ID NO. 68 or 74.

TABLE 2
Antisense target First RNA sequences.

| | SEQ ID | FIRST RNA SEQUENCE | |
|---|---|---|---|
| ref. | NO. | (5′-NNNN-3′) | length |
| C15 | 68 | UUUGGGGUUCGGCUGCCGGGAA | 22 |
| C21 | 74 | AGCGACUGGUGGAAUUGCCUGC | 22 |

As said, such a first RNA sequence is to be combined with a second RNA sequence. As described herein, the skilled person is well capable of designing and selecting a suitable second RNA sequence in order to provide for a first and second RNA sequence that can induce RNA interference when expressed in a cell. Suitable second RNA sequences that can be contemplated are listed below.

TABLE 3
Antisense target Second RNA sequences

| | SEQ ID | SECOND RNA SEQUENCE | |
|---|---|---|---|
| ref. | NO. | (5′-NNNN-3′) | length |
| C15 | 117 | CGGCAGCCGAACCCCAAC | 18 |
| C15 | 153 | CGGCAGCCGAACCCCAA | 17 |
| C21 | 118 | GCAAUUCCACCAGUCGCC | 18 |
| C21 | 154 | GCAAUUCCACCAGUCGC | 17 |

Said first RNA sequence is preferably comprised in a miRNA scaffold, more preferably a miR101 scaffold or a miR451 scaffold, such as shown in the examples. A suitable scaffold comprising a first and second RNA sequence in accordance with the invention can be a sequence such as SEQ ID NO. 119 or 120.

TABLE 4
Antisense pre-miRNA sequences

| | SEQ | first(second) RNA sequence | |
|---|---|---|---|
| | ID | (loop) second(first) RNA | |
| ref. | NO. | sequence [5′-NNNN-3′] | length |
| C15 | 119 | UUUGGGGUUCGGCUGCCGGGAACGGC | 40 |
| | | AGCCGAACCCCAAC | |
| C15 | 155 | UUUGGGGUUCGGCUGCCGGGAACGGC | 39 |
| | | AGCCGAACCCCAA | |
| C21 | 120 | AGCGACUGGUGGAAUUGCCUGCGCAA | 40 |
| | | UUCCACCAGUCGCC | |
| C21 | 156 | AGCGACUGGUGGAAUUGCCUGCGCAA | 39 |
| | | UUCCACCAGUCGC | |

Such first RNA sequences as described above can be comprised in expression cassettes, such as e.g. depicted in FIG. 5a and as depicted in FIGS. 30, 31 and 32. Such first RNA sequences can be comprised in RNA structures that are encoded by expression cassettes, such as depicted in FIGS. 4b, 33 and 34.

Such first and second RNA sequences as described above can be comprised in expression cassettes, such as e.g. depicted in FIG. 5a and as depicted in FIGS. 30, 31 and 32. Such first and second RNA sequences can be comprised in RNA structures that are encoded by expression cassettes, such as depicted in FIGS. 4b, 33 and 34.

Accordingly, targeting these target RNA sequences, utilizing such first and second RNA sequences, was found to be in particular useful for reducing expression of antisense RNA transcripts encoded by the human C9ORF72 gene.

In another embodiment, an expression cassette according to the invention is provided, wherein said first RNA sequence is substantially complementary to a target RNA sequence comprised in exon 2 containing RNA transcripts encoded by the human C9ORF72 gene. Preferably said first RNA sequence is substantially complementary to SEQ ID NO. 32. More preferably said first RNA sequence has a length of 19, 20, 21, or 22 nucleotides. More preferably said first RNA sequence is fully complementary over its entire length with said first RNA target sequence. Most preferably said first RNA sequence has a length of 19, 20, 21, or 22 nucleotides, wherein said first RNA sequence is fully complementary over its entire length with said first RNA target sequence. Preferably, said first RNA strand is selected from either SEQ ID NO. 86 or 91.

TABLE 5
Exon 2 target First RNA sequences

| | SEQ ID | FIRST RNA SEQUENCE | |
|---|---|---|---|
| ref. | NO. | (5′-NNNN-3′) | length |
| C32 | 86 | CCUUACUCUAGGACCAAGAAU | 21 |
| C32 | 91 | GCCUUACUCUAGGACCAAGAAU | 22 |

Such a first RNA sequence is to be combined with a second RNA sequence. As described herein, the skilled person is well capable of designing and selecting a suitable second RNA sequence in order to provide for a first and second RNA sequence that can induce RNA interference when expressed in a cell. Suitable second RNA sequences that can be contemplated are listed below.

TABLE 6
Exon 2 target Second RNA sequences

| | SEQ ID | SECOND RNA SEQUENCE | |
|---|---|---|---|
| ref. | NO. | (5′-NNNN-3′) | length |
| C32 | 121 | UCUUGGUCCUAGAGUAACGGAC | 22 |
| C32 | 122 | UUGGUCCUAGAGUAAGGA | 18 |
| C32 | 157 | UUGGUCCUAGAGUAAGG | 17 |

Said first RNA sequence is preferably comprised in a miRNA scaffold, more preferably a miR101 scaffold or a miR451 scaffold, such as shown in the examples. A suitable scaffold comprising a first and second RNA sequence in accordance with the invention can be a sequence such as SEQ ID NO. 123 or 124.

TABLE 7
Exon 2 target pre-miRNA sequences

| | SEQ | first(second) RNA sequence | |
|---|---|---|---|
| | ID | (loop) second(first) RNA | |
| ref. | NO. | sequence [5′-NNNN-3′] | length |
| C32 | 123 | UCUUGGUCCUAGAGUAACGGACGUCUAUUCUAA | 57 |
| | | AGUCCUUACUCUAGGACCAAGAAU | |
| C32 | 124 | GCCUUACUCUAGGACCAAGAAUUUGGUCCUAGA | 40 |
| | | GUAAGGA | |
| C32 | 158 | GCCUUACUCUAGGACCAAGAAUUUGGUCCUAGA | 39 |
| | | GUAAGG | |

Such first RNA sequences as described above can be comprised in expression cassettes, such as e.g. depicted in FIG. 5a and as depicted in FIGS. 30, 31 and 32. Such first RNA sequences can be comprised in RNA structures that are encoded by expression cassettes, such as depicted in FIGS. 33, 34 and 4b.

Such first and second RNA sequences as described above can be comprised in expression cassettes, such as e.g. depicted in FIG. 4b, 5a and as depicted in FIGS. 30, 31 and 32. Such first and second RNA sequences can be comprised in RNA structures that are encoded by expression cassettes, such as depicted in FIGS. 33 and 34.

Accordingly, targeting these target RNA sequences, utilizing such first and second RNA sequences, was found to be in particular useful for reducing expression of exon 2 containing RNA transcripts encoded by the human C9ORF72 gene.

In another embodiment, an expression cassette according to the invention is provided, wherein said first RNA sequence is substantially complementary to a target RNA sequence comprised in intron 1 containing RNA transcripts encoded by the human C9ORF72 gene. Preferably said first RNA sequence is substantially complementary to SEQ ID NO. 2, SEQ ID NO. 4 or SEQ ID NO. 31. More preferably said first RNA sequence has a length of 19, 20, 21, or 22 nucleotides. More preferably said first RNA sequence is fully complementary over its entire length with said first RNA target sequence. Most preferably said first RNA sequence has a length of 19, 20, 21, or 22 nucleotides, wherein said first RNA sequence is fully complementary over its entire length with said first RNA target sequence. Preferably, said first RNA strand is selected from the SEQ ID NO. 52, 59 and 85.

TABLE 8
Intron 1 target First RNA sequences.

| | SEQ ID | FIRST RNA SEQUENCE | |
|---|---|---|---|
| ref. | NO. | (5′-NNNN-3′) | length |
| C2 | 52 | CACCCUCAGCGAGUACUGUGA | 21 |
| C2 | 58 | UCACCCUCAGCGAGUACUGUGA | 22 |
| C4 | 54 | CUAGCGCGCGACUCCUGAGUU | 21 |
| C4 | 59 | CCUAGCGCGCGACUCCUGAGUU | 22 |
| C31 | 85 | GGUAACUUCAAACUCUUGGGC | 21 |

Such a first RNA sequence is to be combined with a second RNA sequence. As described herein, the skilled person is well capable of designing and selecting a suitable second RNA sequence in order to provide for a first and second RNA sequence that can induce RNA interference when expressed in a cell. Suitable second RNA sequences that can be contemplated are listed below.

TABLE 9
Intron 1 target Second RNA sequences.

| | SEQ ID | SECOND RNA SEQUENCE | |
|---|---|---|---|
| ref. | NO. | (5′-NNNN-3′) | length |
| C2 | 125 | ACAGUACUCGCUGAGGGUUGAC | 22 |
| C2 | 126 | AGUACUCGCUGAGGGUGC | 18 |
| C2 | 159 | AGUACUCGCUGAGGGUG | 17 |
| C4 | 127 | CUCAGGAGUCGCGCGCUGAGGG | 22 |
| C4 | 128 | CAGGAGUCGCGCGCUAGA | 18 |
| C4 | 160 | CAGGAGUCGCGCGCUAG | 17 |
| C31 | 129 | CCAAGAGUUUGAAGUUACCCAG | 22 |

Said first RNA sequence is preferably comprised in a miRNA scaffold, more preferably a miR101 scaffold or a miR451 scaffold, such as shown in the examples. A suitable scaffold comprising a first and second RNA sequence in accordance with the invention can be a sequence such as SEQ ID NO. 128, 129 or 130.

TABLE 10
Intron 1 target pre-miRNA sequences.

| | SEQ | first(second) RNA sequence | |
|---|---|---|---|
| | ID | (loop) second(first) RNA | |
| ref. | NO. | sequence [5′-NNNN-3′] | length |
| C2 | 130 | ACAGUACUCGCUGAGGGUUGACGUCUAUUCUAA | 57 |
| | | AGUCACCCUCAGCGAGUACUGUGA | |
| C2 | 131 | UCACCCUCAGCGAGUACUGUGAAGUACUCGCUG | 40 |
| | | AGGGUGC | |
| C2 | 161 | UCACCCUCAGCGAGUACUGUGAAGUACUCGCUG | 39 |
| | | AGGGUG | |
| C4 | 132 | CUCAGGAGUCGCGCGCUGAGGGCUCUAUUCUAA | 57 |
| | | AUCCUAGCGCGCGACUCCUGAGUU | |
| C4 | 133 | CCUAGCGCGCGACUCCUGAGUUCAGGAGUCGCG | 40 |
| | | CGCUAGA | |
| C4 | 162 | CCUAGCGCGCGACUCCUGAGUUCAGGAGUCGCG | 39 |
| | | CGCUAG | |
| C31 | 134 | CCAAGAGUUUGAAGUUACCCAGCUCUAUUCUAA | 57 |
| | | ACUGGUAACUUCAAACUCUUGGGC | |

Such first RNA sequences as described above can be comprised in expression cassettes, such as e.g. depicted in FIG. 5a and as depicted in FIGS. 30, 31 and 32. Such first RNA sequences can be comprised in RNA structures that are encoded by expression cassettes, such as depicted in FIGS. 4b, 33 and 34.

Accordingly, targeting these target RNA sequences, utilizing such first and second RNA sequences, was found to be in particular useful for reducing expression of intron 1 containing RNA transcripts encoded by the human C9ORF72 gene.

In another embodiment, an expression cassette according to the invention is provided, wherein said first RNA sequence is substantially complementary to a target RNA sequence comprised in exon 11 containing RNA transcripts encoded by the human C9ORF72 gene. Preferably said first RNA sequence is substantially complementary to SEQ ID NO. 46. More preferably said first RNA sequence has a length of 19, 20, 21, or 22 nucleotides. More preferably said first RNA sequence is fully complementary over its entire length with said first RNA target sequence. Most preferably said first RNA sequence has a length of 19, 20, 21, or 22 nucleotides, wherein said first RNA sequence is fully complementary over its entire length with said first RNA target sequence. Preferably, said first RNA sequence is either SEQ ID NO. 104 or 109.

TABLE 11
Exon 11 target First RNA sequences

| | SEQ ID | FIRST RNA SEQUENCE | |
|---|---|---|---|
| ref. | NO. | (5′-NNNN-3′) | length |
| C46 | 104 | UAUCUUCAGGUUCCGAAGAGA | 21 |
| C46 | 109 | CUAUCUUCAGGUUCCGAAGAGA | 22 |

Such a first RNA sequence is to be combined with a second RNA sequence. As described herein, the skilled person is well capable of designing and selecting a suitable second RNA sequence in order to provide for a first and second RNA sequence that can induce RNA interference when expressed in a cell. Suitable second RNA sequences that can be contemplated are listed below.

TABLE 12
Exon 11 target Second RNA sequences.

| | SEQ ID | SECOND RNA SEQUENCE | |
|---|---|---|---|
| ref. | NO. | (5′-NNNN-3′) | length |
| C46 | 135 | UCUUCGGAACCUGAAGAUUGAC | 22 |
| C46 | 136 | UUCGGAACCUGAAGAUAC | 18 |
| C46 | 163 | UUCGGAACCUGAAGAUA | 17 |

Said first RNA sequence is preferably comprised in a miRNA scaffold, more preferably a miR101 scaffold or a miR451 scaffold, such as shown in the examples. A suitable scaffold comprising a first and second RNA sequence in accordance with the invention can be a sequence such as SEQ ID NO. 133 or 134.

TABLE 13
Exon 11 target pre-miRNA sequences.

| | SEQ | first(second) RNA sequence | |
|---|---|---|---|
| | ID | (loop) second(first) RNA | |
| ref. | NO. | sequence [5′-NNNN-3′] | length |
| C46 | 137 | UCUUCGGAACCUGAAGAUUGACGUCUAUU | 57 |
| | | CUAAAGUUAUCUUCAGGUUCCGAAGAGA | |
| C46 | 138 | CUAUCUUCAGGUUCCGAAGAGAUUCGGAA | 40 |
| | | CCUGAAGAUAC | |
| C46 | 164 | CUAUCUUCAGGUUCCGAAGAGAUUCGGAA | 39 |
| | | CCUGAAGAUA | |

Such first RNA sequences as described above can be comprised in expression cassettes, such as e.g. depicted in FIG. 4b, 5a and as depicted in FIGS. 30, 31 and 32. Such first RNA sequences can be comprised in RNA structures that are encoded by expression cassettes, such as depicted in FIGS. 33 and 34.

Such first and second RNA sequences as described above can be comprised in expression cassettes, such as e.g. depicted in FIG. 5a and as depicted in FIGS. 30, 31 and 32. Such first and second RNA sequences can be comprised in RNA structures that are encoded by expression cassettes, such as depicted in FIGS. 4b, 33 and 34.

Accordingly, targeting these target RNA sequences, utilizing such first and second RNA sequences, was found to be in particular useful for reducing expression of exon 11 containing RNA transcripts encoded by the human C9ORF72 gene.

As described above, and as shown in the examples, these target sequences were found to be in particular suitable for reducing C9ORF72 gene expression via an RNAi approach that utilizes an expression cassette encoding a first RNA sequence and a second RNA sequence wherein the first and second RNA sequence are substantially complementary, wherein the first RNA sequence has a sequence length of at least 19 nucleotides and is substantially complementarity to a target RNA sequence comprised in an RNA encoded by a human C9ORF72 gene.

Moreover, and in further embodiments, one or more expression cassettes are provided for combined targeting of said target RNA sequences. In particularly useful was found to be the combination of targeting sense and antisense transcripts. Hence, combined targeting of RNA target sequences comprised in antisense RNA encoded by a human C9ORF72 gene, with targeting of RNA target sequences comprised in either an exon 2, exon 11 or intron 1 containing RNA encoded by the human C9ORF72 gene is contemplated in the invention. Such combined targeting is to reduce hexanucleotide repeat containing transcripts and/or expressed DPR polypeptides even further.

Combined targeting of RNA target sequences can be obtained by providing two separate expression cassettes. Examples of expression cassettes for a miR451 scaffold and a miR101 scaffold are depicted in FIGS. 30 and 31, respectively. Alternatively, and preferably, one expression cassette is provided that is to encode for each target a first RNA sequence combined with a second RNA sequence, such an expression cassette thus expressing a single RNA transcript comprising two separate first RNA sequences that can be processed by the cell to provide for two separate guide sequences, each separate guide sequence targeting one of the two targets, i.e. a sense target RNA sequence and an antisense target RNA sequence. As shown in the examples, first and second RNA sequences comprised in pre-miRNA or pri-miRNA structures are suitable to be comprised in such single RNA transcripts (FIG. 32).

Hence, in one embodiment, one or more expression cassettes are provided for combined targeting of SEQ ID NO.15, with one of the target RNA sequences selected from the group consisting of SEQ ID NO. 2, 4, 31, 32 and 46. In another embodiment, one or more expression cassettes in accordance with the invention are provided for combined targeting of SEQ ID NO. 21 with one of the target RNA sequences selected from the group consisting of SEQ ID NO. 2, 4, 31, 32 and 46.

Preferably a pol II promoter is used, such as a CAG promoter (i.a. Miyazaki et al. Gene. 79 (2): 269-77; Niwa, Gene. 108 (2): 193-9), a PGK promoter, or a CMV promoter (Such as depicted e.g. in FIG. 2 of WO2016102664, which is herein incorporated by references). As ALS and/or FTD affects neurons, it may in particularly be useful to use a neurospecific promoter. Examples of suitable neurospecific promoters are Neuron-Specific Enolase (NSE), human synapsin 1, caMK kinase and tubuline (Hioki et al. Gene Ther. 2007 June; 14(11):872-82). Other suitable promoters that can be contemplated are inducible promoters, i.e. a promoter that initiates transcription only when the host cell is exposed to some particular stimulus.

Said expression cassettes according to the invention can be transferred to a cell, using e.g. transfection methods. Any suitable means may suffice to transfer an expression cassette according to the invention. Preferably, gene therapy vectors are used that stably transfer the expression cassette to the cells such that stable expression of the double stranded RNAs that induce sequence specific inhibition of the C9ORF72 gene as described above can be achieved. Suitable vectors may be lentiviral vectors, retrotransposon based vector systems, or AAV vectors. It is understood that as e.g. lentiviral vectors carry an RNA genome, the RNA genome will encode for the said expression cassette such that after transduction of a cell, the said DNA sequence and said expression cassette is formed. Preferably a viral vector is used such as AAV. Preferably the AAV vector that is used is an AAV vector of serotype 5. AAV of serotype 5 (also referred to as AAV5) may be in particularly useful for transducing human neurons and human astrocytes such as shown in the examples. Thus, AAV5 can efficiently transduce different human cell types of the CNS including FBN, dopaminergic neurons, motor neurons and astrocytes and is therefore a suitable vector candidate to deliver therapeutic genes to the CNS to treat neurogenerative diseases, including but not limited to the treatment of ALS and/or FTD via targeting e.g. C9ORF72 as described herein. The production of AAV vectors comprising any expression cassette of interest is well described in; WO2007/046703, WO2007/148971, WO2009/014445, WO2009/104964, WO201 1/122950, WO2013/0361 18, which are incorporated herein in its entirety.

AAV sequences that may be used in the present invention for the production of AAV vectors, e.g. produced in insect or mammalian cell lines, can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. AAV capsids may consist of VP1, VP2 and VP3, but may also consist of VP1 and VP3.

In another embodiment, a host cell is provided comprising the said DNA sequence or said expression cassette according to the invention. For example, the said expression cassette or DNA sequence may be comprised in a plasmid contained in bacteria. Said expression cassette or DNA sequence may also be comprised in a production cell that produces e.g. a viral vector. Said expression cassette may also be provided in a baculovirus vector.

As shown in the example section, and as explained above, the double stranded RNA according to the invention, the DNA sequence according to invention, the expression cassette according to the invention and the gene therapy vector according to the invention are for use in a medical treatment, in particular for use in the treatment of ALS and/or FTD disease.

Said first and second RNA sequences in accordance with the invention, when expressed in a cell preferably reduce expression of RNA encoded by a human C9ORF72 gene both in the cell nucleus as in the cytoplasm. In particularly it was found that miRNA-scaffolds such as based on miR101 and miR451 were in particular useful as scaffolds for inducing sequence specific knock down of a selected target RNA sequence comprised in nuclear RNA targets, e.g. comprised in intronic sequences. It is understood that such uses may not necessarily be restricted to targeting C9ORF72 transcripts in the nucleus, but may be in general useful against other nuclear transcripts as well. In a further embodiment, such nuclear RNA targeting preferably comprises nuclear RNAs expressed in the CNS, preferably in neuronal cells, most preferably in human neuronal cells.

Accordingly said gene therapy vector when said first and second RNA sequences are expressed in a cell can advantageously reduce expression of C9 RAN protein levels. Furthermore, said first and second RNA sequences when expressed in a cell can also advantageously reduce expression of G4C2 foci and/or G2C4 foci.

The invention also provides for a medical treatment, said medical treatment when using an AAV vector (or likewise for any suitable gene therapy vector) comprising delivery of AAV vector in accordance with the invention to the CNS. Preferably, said medical treatment, utilizing a gene therapy vector in accordance with the invention, comprises transfer of the vector to a motorneuron, as affected cells in the CNS include motorneurons. Preferably, said medical treatment may also comprise transfer of the vector to a human frontal brain neuron and/or anterior brain neuron. Preferably, said gene therapy vector is administered to the spinal cord. In another embodiment, said gene therapy vector in accordance with the invention is administered to the frontal lobe and/or anterior temporal lobe.

Said delivery to the CNS may comprise intraparenchymal injections (Samaranch et al., Gene Ther. 2017 April; 24(4):253-261). Said delivery may also comprise delivery to the cerebrospinal fluid upon which affected CNS regions may be effectively transduced as the vector via the cerebrospinal fluid can reach affected areas in the disease, such as cortical areas and the spinal cord, via diffusion. Said delivery in further embodiments may thus comprise intrathecal (e.g. WO2015060722; Bailey et al., Mol Ther Methods Clin Dev. 2018 Feb. 15; 9:160-171) or subpial (Miyanohara et al., Mol Ther Methods Clin Dev. 2016 Jul. 13; 3:16046.) injections of the vector. Said delivery may also comprise intracerebroventricular (ICV) or intrastriatal injections. Preferably, the delivery does not comprise intraparenchymal injections, as such delivery routes may have a risk of inducing injury. Said delivery may also comprise a combination of said delivery methods. For example, intrathecal or subpial injection may be combined within tracerebroventricular and/or intrastriatal injections. Intrathecal or subpial injection may also be combined with intraparenchymal injections.

Such delivery methods representing an efficient way to deliver the gene therapy vector to the CNS, including affected cortical and spinal cord regions and to target the neurons. Such injections are preferably carried out through MRI-guided injections. Said methods of treatments are in particular useful for human subjects having ALS and/or FTD. It is understood that the treatment of ALS and/or FTD involves human subjects having ALS and/or FTD including human subjects having a genetic predisposition of developing ALS and/or FTD that do not yet show signs of the disease. Hence, the treatment of human subjects with ALS and/or FTD includes the treatment of any human subject carrying an C9ORF72 allele with hexanucleotide repeats.

### EXAMPLES

### Reduced C9ORF72 Levels Detected by RNA-Seq in C9-ALS Patients

The human C9ORF72 gene consists of 12 exons that can be transcribed in three different transcript variants (V1, V2, V3) (FIG. 1). Prudencio et al. (Nat Neurosci. 2015 August; 18(8):1175-82) have investigated the transcriptomes of cerebellum and frontal cortex from 7 controls and 8 patients with C9ORF72-associated ALS. The accompanying RNAseq data from that study has been uploaded to the NCBI Gene Expression Omnibus under accession number GSE67196 and was used in our analysis (FIGS. 2 and 3).

Notably, we found that C9ORF72 is expressed at higher levels (˜2 fold) in the cerebellum compared to cortex in both C9ORF72-ALS patients and controls. It was also found that C9ORF72 mRNA expression is consistently reduced in both cerebellum and cortex of C9ORF72ALS patients. In both cerebellum and cortex, the relative expression of V1, V2 and V3 in patients and controls were found to be similar, suggesting that the reduction of C9ORF72 mRNA levels in C9-ALS patients is not variant specific.

From a wild-type RNA, the C9ORF72 Intron 1 should be spliced out and degraded. In a G4C2 repeat containing transcript defective splicing may result in accumulation of transcripts. Read alignments from C9-ALS patients and controls were compared to investigate the sequence conservation of intronic and exonic regions of C9ORF72 RNA transcripts (FIG. 3b). The read depth in exon 1a, exon 1b, intron 1, exon 2 and exon 11 was estimated by correcting the total number of reads by the area size. We found a complete coverage of exon 2 to exon 11, though read depth in exon 1a, exon 1b and intronic regions was very low in both C9-ALS and control groups. Exonic regions from exon 2 to exon 11 were less covered in patients while read depth for intron 1 in the patients was comparable with controls. To estimate the relative coverage of the exons and introns, the ratio between the number of reads in C9-ALS patients and controls was determined (FIG. 1e). All C9ORF72 exonic regions were about twofold lower expressed in C9-ALS patients. Intron 1 had the same number of reads in patient samples compared to controls, while intron 2-4 was 1.4 times higher in C9-ALS patients than controls. Introns 5, 6 and 7 were excluded as these could potentially be 3′UTR of the short C9ORF72 variant and coverage of intron 8, 9 and 10 was not increased in C9-ALS patients. Similarly, in the frontal cortex samples of C9-ALS patients the coverage ratio between intronic and exonic regions was increased (fig. S1). Thus, although exonic regions were expressed twofold lower in C9-ALS patients, this was not the case for intron 1-4, suggesting that intronic C9ORF72 reads are relatively overexpressed in C9-ALS patients. Our data indicate that the higher amount of sequences from intron 1-4 in mRNA could be due to aberrant transcription.

Design of miRNAs Targeting Conserved C9ORF72 Regions
We selected target sites for a total silencing approach in exon 2 and 11, as target sites in these regions are present in all sense transcripts, spliced and unspliced (see FIG. 1). Further target sequences were selected in intron 1, intron 2 and in antisense transcripts. Sequences in intron 1, exon 2 and exon 11 of C9ORF72 were selected that were highly conserved between human, non-human primates and mouse. Furthermore, conservation of target sequences in intron 1, intron 2 and exon 1 and exon 11 was confirmed by alignment of target sequences with transcriptome sequence data. Target sequences were based on 21 or 22 nucleotide sequence lengths as the RNAi design involved miRNA scaffolds based on miR101 and miR451. Selected target sequences are listed below.

TABLE 14
Target sequences.

| | SEQ | Position | | |
|---|---|---|---|---|
| | ID | Refseq | TARGET RNA SEQUENCE | |
| ref. | NO. | NG_031977 | (5′-NNNN-3′) | Length |
| C1 | 1 | 5177-5197 | CCCACCCUCUCUCCCCACUAC | 21 |
| C2 | 2 | 5204-5225 | UCACAGUACUCGCUGAGGGUGA | 22 |
| C3 | 3 | 5267-5287 | GAGGGAAACAACCGCAGCCUG | 21 |
| C4 | 4 | 5301-5322 | AACUCAGGAGUCGCGCGCUAGG | 22 |
| C5 | 5 | 5308-5329 | AGUCGCGCGCUAGGGGCCGGGG | 22 |
| C6 | 6 | 5318-5338 | CUAGGGGCCGGGGCCGGGGCC | 21 |
| C7 | 7 | 5321-5341 | GGGGCCGGGGCCGGGGCCGGG | 21 |
| C8 | 8 | 5186-5207 | UCUCCCCACUACUUGCUCUCAC | 22 |
| C9 | 9 | 5208-5228 | AGUACUCGCUGAGGGUGAACAA | 22 |
| C10 | 10 | 5236-5257 | ACCUGAUAAAGAUUAACCAGAA | 22 |
| C11 | 11 | 5262-5293 | ACAAGGAGGGAAACAACCGCAG | 22 |
| C12 | 12 | 5423-5444 | UCACUCACCCACUCGCCACCGC | 22 |
| C13 | 13 | 5437-5458 | AGGAUGCCGCCUCCUCACUCAC | 22 |
| C14 | 14 | 5453-5474 | CAAACAGCCACCCGCCAGGAUG | 22 |
| C15 | 15 | 5471-5492 | UUCCCGGCAGCCGAACCCCAAA | 22 |
| C16 | 16 | 5490-5511 | CCGCUUCUACCCGCGCCUCUUC | 22 |
| C17 | 17 | 5514-5535 | UGCGUCGAGCUCUGAGGAGAGC | 22 |
| C18 | 18 | 5532-5553 | UGAGAGGGAAAGUAAAAAUGCG | 22 |
| C19 | 19 | 5557-5578 | CGACACCCAGCUUCGGUCAGAG | 22 |
| C20 | 20 | 5577-5598 | AGUCGCUAGAGGCGAAAGCCCG | 22 |
| C21 | 21 | 5592-5613 | GCAGGCAAUUCCACCAGUCGCU | 22 |
| C22 | 22 | 5847-5868 | UCGGGGUUCGCUAGGAACCCGA | 22 |
| C23 | 23 | 5886-5907 | AGGAGAUCAUGCGGGAUGAGAU | 22 |
| C24 | 24 | 5915-5936 | UGGAGACGCCUGCACAAUUUCA | 22 |
| C25 | 25 | 5953-5974 | AGUGGUGAUGACUUGCAUAUGA | 22 |
| C26 | 26 | 5983-6004 | AUGCAAGUCGGUGUGCUCCCCA | 22 |
| C27 | 27 | 6008-6029 | UGUGGGACAUGACCUGGUUGCU | 22 |
| C28 | 28 | 6033-6054 | CAGCUCCGAGAUGACACAGACU | 22 |
| C29 | 29 | 6073-6094 | AUUGUGACUUGGGCAUCACUUG | 22 |
| C30 | 30 | 6092-6112 | UUGACUGAUGGUAAUCAGUUG | 21 |
| C31 | 31 | 7315-7335 | GCCCAAGAGUUUGAAGUUACC | 21 |
| C32 | 32 | 11852-11873 | AUUCUUGGUCCUAGAGUAAGGC | 22 |
| C33 | 33 | 11853-11873 | UUCUUGGUCCUAGAGUAAGGC | 21 |
| C34 | 34 | 11903-11924 | CUUCUCAGUGAUGGAGAAAUAA | 22 |
| C35 | 35 | 11935-11956 | CCAACCACACUCUAAAUGGAGA | 22 |
| C36 | 36 | 12168-12189 | GGAAGAAUAUGGAUGCAUAAGA | 22 |
| C37 | 37 | 11776-11797 | AGCUGUUGCCAAGACAGAGAUU | 22 |
| C38 | 38 | 11779-11800 | UGUUGCCAAGACAGAGAUUGCU | 22 |
| C39 | 39 | 11782-11803 | UGCCAAGACAGAGAUUGCUUUA | 22 |
| C40 | 40 | 11785-11806 | CAAGACAGAGAUUGCUUUAAGU | 22 |
| C41 | 41 | 11787-11808 | AGACAGAGAUUGCUUUAAGUGG | 22 |
| C42 | 42 | 11863-11884 | UAGAGUAAGGCACAUUUGGGCU | 22 |
| C43 | 43 | 11916-11937 | GAGAAAUAACUUUUCUUGCCAA | 22 |
| C44 | 44 | 11923-11944 | AGUGUGGUUGGCAAGAAAAGUU | 22 |
| C45 | 45 | 11932-11953 | UGCCAACCACACUCUAAAUGGA | 22 |
| C46 | 46 | 30470-30491 | UCUCUUCGGAACCUGAAGAUAG | 22 |
| C47 | 47 | 30494-30514 | CUUGAUUUAACAGCAGAGGGC | 21 |
| C48 | 48 | 30517-30537 | UCUUAACAUAAUAAUGGCUCU | 21 |
| C49 | 49 | 31112-31133 | GAGCUUGAACAUAGGAUGAGCU | 22 |
| C50 | 50 | 32114-32135 | AAUACUACCUUGUAGUGUCCCA | 22 |

Table 14. C1-C11 and C22-C31 correspond with target RNA sequences of transcripts encoded by the human C9ORF72 gene of intron 1; C12-C21 correspond with target RNA sequences in antisense transcripts encoded by the human C9ORF72 gene; C32-C45 correspond with target RNA sequences of transcripts encoded by the human C9ORF72 gene of exon 2; C46-050 correspond with target RNA sequences of transcripts encoded by the human C9ORF72 gene of exon 11. Selected target RNA sequences listed are either 21 or 22 nucleotides in length, or both. For a target RNA sequence either a miRNA scaffold was designed based on a 21 nucleotide target RNA sequence (101-scaffold) or a 22 nucleotide target RNA sequence (451 scaffold). For C2, C4, C5, C8-C29, C32, C34-C36, C46, C49, and C50 both a scaffold based on miR101 and miR451 was designed, wherein the target RNA sequence for the 101 scaffold represents the first 5′ 21 nucleotides listed. The first RNA sequences that were used to replace the endogenous guide strand sequence in the miRNA scaffolds are listed below. The miC sequences were incorporated into human pri-miRNA miR-101-1 or miR-451 scaffold sequences. 200 nt 5′ and 3′ flanking regions were included and the mfold program (http://unafold.rna.albany.edu/?q=mfold) was used with standard settings to determine whether the miC candidates are folded into the secondary structures as depicted in FIG. 4b. Complete sequences were ordered from GeneArt gene synthesis (Invitrogen) and were subsequently cloned into an expression vector containing the CMV immediate-early enhancer fused to chicken (3-actin (CAG) promoter (Inovio, Plymouth Meeting, PA).

TABLE 15
First RNA sequences.

| | SEQ ID | FIRST RNA SEQUENCE | |
|---|---|---|---|
| ref. | NO. | (5′-NNNN-3′) | Length |
| C1 | 51 | GUAGUGGGGAGAGAGGGUGGG | 21 |
| C2 | 52 | CACCCUCAGCGAGUACUGUGA | 21 |
| C3 | 53 | CAGGCUGCGGUUGUUUCCCUC | 21 |
| C4 | 54 | CUAGCGCGCGACUCCUGAGUU | 21 |
| C5 | 55 | CCCGGCCCCUAGCGCGCGACU | 21 |
| C6 | 56 | GGCCCCGGCCCCGGCCCCUAG | 21 |
| C7 | 57 | CCCGGCCCCGGCCCCGGCCCC | 21 |
| C2 | 58 | UCACCCUCAGCGAGUACUGUGA | 22 |
| C4 | 59 | CCUAGCGCGCGACUCCUGAGUU | 22 |
| C5 | 60 | CCCGGCCCCUAGCGCGCGACUC | 22 |
| C8 | 61 | GUGAGAGCAAGUAGUGGGGAGA | 22 |
| C9 | 62 | UUGUUCACCCUCAGCGAGUACU | 22 |
| C10 | 63 | UUCUGGUUAAUCUUUAUCAGGU | 22 |
| C11 | 64 | CUGCGGUUGUUUCCCUCCUUGU | 22 |
| C12 | 65 | GCGGUGGCGAGUGGGUGAGUGA | 22 |
| C13 | 66 | GUGAGUGAGGAGGCGGCAUCCU | 22 |
| C14 | 67 | CAUCCUGGCGGGUGGCUGUUUG | 22 |
| C15 | 68 | UUUGGGGUUCGGCUGCCGGGAA | 22 |
| C16 | 69 | GAAGAGGCGCGGGUAGAAGCGG | 22 |
| C17 | 70 | GCUCUCCUCAGAGCUCGACGCA | 22 |
| C18 | 71 | CGCAUUUUUACUUUCCCUCUCA | 22 |
| C19 | 72 | CUCUGACCGAAGCUGGGUGUCG | 22 |
| C20 | 73 | CGGGCUUUCGCCUCUAGCGACU | 22 |
| C21 | 74 | AGCGACUGGUGGAAUUGCCUGC | 22 |
| C22 | 75 | UCGGGUUCCUAGCGAACCCCGA | 22 |
| C23 | 76 | AUCUCAUCCCGCAUGAUCUCCU | 22 |
| C24 | 77 | UGAAAUUGUGCAGGCGUCUCCA | 22 |
| C25 | 78 | UCAUAUGCAAGUCAUCACCACU | 22 |
| C26 | 79 | UGGGGAGCACACCGACUUGCAU | 22 |
| C27 | 80 | AGCAACCAGGUCAUGUCCCACA | 22 |
| C28 | 81 | AGUCUGUGUCAUCUCGGAGCUG | 22 |
| C29 | 82 | AAGUGAUGCCCAAGUCACAAU | 21 |
| C29 | 83 | CAAGUGAUGCCCAAGUCACAAU | 22 |
| C30 | 84 | CAACUGAUUACCAUCAGUCAA | 21 |
| C31 | 85 | GGUAACUUCAAACUCUUGGGC | 21 |
| C32 | 86 | CCUUACUCUAGGACCAAGAAU | 21 |
| C33 | 87 | GCCUUACUCUAGGACCAAGAA | 21 |
| C34 | 88 | UAUUUCUCCAUCACUGAGAAG | 21 |
| C35 | 89 | CUCCAUUUAGAGUGUGGUUGG | 21 |
| C36 | 90 | CUUAUGCAUCCAUAUUCUUCC | 21 |
| C32 | 91 | GCCUUACUCUAGGACCAAGAAU | 22 |
| C34 | 92 | UUAUUUCUCCAUCACUGAGAAG | 22 |
| C35 | 93 | UUCUCCAUUUAGAGUGUGGUUG | 22 |
| C36 | 94 | UUAUGCAUCCAUAUUCUUCCUU | 22 |
| C37 | 95 | AAUCUCUGUCUUGGCAACAGCU | 22 |
| C38 | 96 | AGCAAUCUCUGUCUUGGCAACA | 22 |
| C39 | 97 | UAAAGCAAUCUCUGUCUUGGCA | 22 |
| C40 | 98 | ACUUAAAGCAAUCUCUGUCUUG | 22 |
| C41 | 99 | CCACUUAAAGCAAUCUCUGUCU | 22 |
| C42 | 100 | AGCCCAAAUGUGCCUUACUCUA | 22 |
| C43 | 101 | UUGGCAAGAAAAGUUAUUUCUC | 22 |
| C44 | 102 | AACUUUUCUUGCCAACCACACU | 22 |
| C45 | 103 | UCCAUUUAGAGUGUGGUUGGCA | 22 |
| C46 | 104 | UAUCUUCAGGUUCCGAAGAGA | 21 |
| C47 | 105 | GCCCUCUGCUGUUAAAUCAAG | 21 |
| C48 | 106 | AGAGCCAUUAUUAUGUUAAGA | 21 |
| C49 | 107 | GCUCAUCCUAUGUUCAAGCUC | 21 |
| C50 | 108 | GGGACACUACAAGGUAGUAUU | 21 |
| C46 | 109 | CUAUCUUCAGGUUCCGAAGAGA | 22 |
| C49 | 110 | AGCUCAUCCUAUGUUCAAGCUC | 22 |
| C50 | 111 | UGGGACACUACAAGGUAGUAUU | 22 |

Table 15. C1-C11 and C22-C31 correspond with first RNA sequences targeting the human C9ORF72 gene of intron 1; C12-C21 correspond with first RNA sequences targeting antisense transcripts encoded by the human C9ORF72 gene; C32-C45 correspond with first RNA sequences targeting transcripts encoded by the human C9ORF72 gene of exon 2; C46-050 correspond with first RNA sequences targeting transcripts encoded by the human C9ORF72 gene of exon 11. Selected first RNA sequences listed are either 21 or 22 nucleotides in length. miRNA scaffold design was based on a 21 nucleotide target RNA sequence (101-scaffold) or a 22 nucleotide target RNA sequence (451 scaffold). For C2, C4, C5, C8-C29, C32, C34-C36, C46, C49, and C50 both a scaffold based on 101 and 451 was designed (See i.a. FIGS. 33 and 34 and table 16 below). The miC sequences were embedded in the pri-miR-101 and pri-miR-451 scaffold because we observed that these scaffolds can produce high amount of active guide strands. The pri-miC-101 and pri-miC-451 endogenous structures are predicted to produce mature miC lengths of 21 nt and 22 nt respectively (FIG. 4b). The miC constructs were expressed by the synthetic CMV early enhancer/chicken β actin (CAG) promotor (FIG. 5a). This promoter is known to drive stable and high expression of a transgene and to be highly active in the CNS.

TABLE 16
RNA sequences as encoded by expression
cassettes and comprised in expressed
transcripts and as depicted in FIGS. 33 and 34.

| | SEQ | first(second) RNA sequence | |
|---|---|---|---|
| | ID | (loop) second(first) RNA | |
| ref. | NO. | sequence [5′-NNNN-3′] | length |
| C15 | 139 | CUUGGGAAUGGCAAGGUUUGGGGUUCGGCU | 72 |
| | | GCCGGGAACGGCAGCCGAACCCCAACACUU | |
| | | GCUAUACCCAGA | |
| C21 | 140 | CUUGGGAAUGGCAAGGAGCGACUGGUGGAA | 72 |
| | | UUGCCUGCGCAAUUCCACCAGUCGCCACUU | |
| | | GCUAUACCCAGA | |
| C32 | 141 | UGCCCUGGCUUCUUGGUCCUAGAGUAACGG | 75 |
| | | ACGUCUAUUCUAAAGUCCUUACUCUAGGAC | |
| | | CAAGAAUGGAUGGCA | |
| C32 | 142 | CUUGGGAAUGGCAAGGGCCUUACUCUAGGA | 72 |
| | | CCAAGAAUUUGGUCCUAGAGUAAGGAUCUU | |
| | | GCUAUACCCAGA | |
| C2 | 143 | UGCCCUGGCCACAGUACUCGCUGAGGGUUG | 75 |
| | | ACGUCUAUUCUAAAGUCACCCUCAGCGAGU | |
| | | ACUGUGAGGAUGGCA | |
| C2 | 144 | CUUGGGAAUGGCAAGGUCACCCUCAGCGAG | 72 |
| | | UACUGUGAAGUACUCGCUGAGGGUGCUCUU | |
| | | GCUAUACCCAGA | |
| C4 | 145 | UGCCCUGGCACUCAGGAGUCGCGCGCUGAG | 75 |
| | | GGCUCUAUUCUAAAUCCUAGCGCGCGACUC | |
| | | CUGAGUUGGAUGGCA | |
| C4 | 146 | CUUGGGAAUGGCAAGGCCUAGCGCGCGACU | 72 |
| | | CCUGAGUUCAGGAGUCGCGCGCUAGAUCUU | |
| | | GCUAUACCCAGA | |
| C31 | 147 | UGCCCUAGACCCAAGAGUUUGAAGUUACCC | 75 |
| | | AGCUCUAUUCUAAACUGGUAACUUCAAACU | |
| | | CUUGGGCGGAUGGCA | |
| C46 | 148 | UGCCCUGGCCUCUUCGGAACCUGAAGAUUG | 75 |
| | | ACGUCUAUUCUAAAGUUAUCUUCAGGUUCC | |
| | | GAAGAGAGGAUGGCA | |
| C46 | 149 | CUUGGGAAUGGCAAGGCUAUCUUCAGGUUC | 72 |
| | | CGAAGAGAUUCGGAACCUGAAGAUACUCUU | |
| | | GCUAUACCCAGA | |

In Vitro Testing of miC-101 and miC-451 Constructs on Reporter Systems
To test the efficacy of the miC candidates, we designed Luc reporters bearing complementary C9ORF72 target regions fused to the renilla luciferase (RL) gene (FIG. 2d). Target sequences were synthesized (GeneArt) and cloned in the 3′UTR of the renilla luciferase (RL) gene of the psiCHECK-2 vector (Promega, Madison, Wis.). The firefly luciferase (FL) gene was also expressed in this vector and served as internal control (FIG. 5b). Co-transfections of reporters and miC constructs were carried out in 293T cells using Lipofectamine using standard culture and transfection conditions and in accordance with manufacturer's instructions. 48 hours post-transfection, cells were lysed in passive lysis buffer (Promega) at room temperature, and FL and RL activities were measured in lysate with the Dual-Luciferase Reporter Assay System (Promega). Relative luciferase activity was calculated as the ratio between RL and FL activities.

We first performed a prescreening for all the miC expression constructs by co-transfection with the corresponding Luc reporters in a 1:1 ratio. Of the miC variants designed to target the sense intronic transcripts, miC2_101 and miC4_101 showed a moderate knockdown (˜40%) and miC31_101 showed a strong knockdown (˜80%) (FIG. 6). These were selected for further optimization. Amongst candidates predicted to target total C9ORF72, miC32_101, miC33_101, miC38_451, miC39_451, miC40_451 and miC43_451 targeting exon 2 showed a strong knockdown of >80% (FIG. 7a). Similarly, miC46_101, miC49_451 and miC50_451 targeting exon 11 induced a strong knockdown (>80%) (FIG. 7b). Dilution of the selected miC candidates demonstrated that the most effective candidates were miC31_101 against intron 1 (FIG. 8a), miC32_101 against exon 2 (FIG. 8 b) and miC46_101 against exon 11 (FIG. 9a). For the antisense C9ORF72 transcript, miC15_451* and miC21_451* were selected as the most effective candidates with a knockdown efficiency of ˜70% (FIG. 7c). Both miC15_451* and miC21_451* showed an equal dose dependent knockdown (FIG. 9b).

Our data indicate that intron 1 is a difficult target region as all miC candidates in the highly structured repeat region between exon 1a and 1b did not induce a knockdown as strong as observed for the other target regions. The most potent knock down was observed with miC candidates downstream of exon 1b and in exonic regions 2 and 11. Nevertheless, a moderate knockdown of the G4C2 repeat containing sequences can be sufficient, miC2_101 and miC4_101 are considered suitable candidates to target the repeat containing transcripts in C9-ALS patients.

Bidirectional targeting, i.e. targeting of both sense and antisense transcripts as expressed from C9ORF72, is possible by introduction of e.g. a second miRNA scaffold (see FIG. 5a), wherein both miR451 and miR101 scaffolds are combined in a single expressed transcript. The region in the vicinity of the G4C2 repeat of C9ORF72 is transcribed in both sense and antisense transcripts and both strands have been linked to toxicity. Therefore, simultaneously targeting both strands could potentially add to therapeutic benefit. To investigate the feasibility for this approach using miRNAs we made concatenate constructs expressing two hairpins predicted to target both transcripts under control of the CAG-promotor. The first hairpin from the concatenated hairpin miRNA construct was in a miR-451 scaffold and targets the antisense transcript. The second hairpin was in a miR-101 scaffold against the sense transcripts. The most effective candidates on luc reporters for intron 1 sense and antisense were selected. The miC15*+31 construct was designed to express miC15_451* and miC31_101 and was tested on intron 1 and antisense reporters (FIGS. 10a and 10b). A silencing of up to 60% was observed on the intron 1 sense and on the antisense reporter. Similarly, miC21*+31 expressing miC21_451* and miC31_101 was made and tested and up to 60% knockdown was observed on both reporter constructs (FIGS. 11a and 11b). Both constructs showed a dose dependent reduction of the intron 1 sense and antisense reporters containing Luc. Our data demonstrates that two different miC can be properly processed and are active when expressed from a single promotor. Hence, a bidirectional miRNA-based approach to simultaneously target the sense and antisense transcripts of C9ORF72 is feasible, using e.g. two miC variants expressed in a concatenated fashion.

### Endogenous Knockdown of C9ORF72 Expression in HEK293T Cells by miC Variants

We next investigated whether the selected miC candidates reduce the endogenous levels of C9ORF72 in HEK293T cells. RNA was isolated from cells using standard procedures, DNA removed using DNase and endogenous levels of C9ORF72 transcipts was determined as described previously and gene expression levels were normalized to GADPH (Liu et al. (2017). Cell Chem. Biol. 24: 141-148).

Cells were transfected with the selected miC candidates and endogenous levels of C9ORF72 mRNA were determined 2 days post-transfection by RT-qPCR. As HEK293T cells lack the G4C2 expansion linked to the C9ORF72 pathology we first determined the expression of total C9ORF72 and intronic C9ORF72 mRNA. We found abundant expression of total C9ORF72, while the intronic C9ORF72 was detectable but at very low levels (FIG. 12a). For miC candidates in miR-101 scaffold, total C9ORF72 mRNA was reduced up to 50% by miC32_101, miC33_101 and miC46_101 (FIG. 12b). The intronic C9ORF72 transcripts were also decreased by ˜25% (FIG. 13). miC2_101 and miC4_101 targeting intron 1 were less effective in lowering the total C9ORF72 but the efficacy on intronic C9ORF72 was comparable to miC candidates targeting total C9ORF72. Thus, both candidates are useful to target the repeat containing transcripts without significantly changing the total C9ORF72 expression. miC31_101 targeting intron 1 showed the best efficacy for the intronic C9ORF72 (40%) but despite its intronic localization, the total C9ORF72 RNA level was also reduced by ˜40% (FIGS. 12b and 13). Reduction of C9ORF72 was also observed by the selected miC candidates in miR-451 scaffold but their efficacy was slightly lower. Altogether, we demonstrated reduction of endogenous levels of C9ORF72 in HEK293T cells, confirming that the miC candidates are functional in cells.

Different Processing Pattern from miR-101 and miR-451 Scaffolds
To assess the processing of the miC candidates, we analyzed the mature miC lengths and sequence composition of the guide and passenger strands by next-generation sequencing (NGS) for small transcriptome analysis (FIGS. 14 and 15). NGS was performed on small RNAs isolated from HEK293T cells that were transfected with the selected miC constructs. For each sample, we obtained between 15-30 million small RNA reads that were subsequently adaptor-trimmed and aligned against the corresponding reference sequence. All reads shorter than 10 nucleotide (nt), longer than 45 nt, or represented less than 10 times were excluded from the analysis.

miR-101 is processed into a miRNA duplex, first by Drosha cleavage at 3′end and then by Dicer cleavage at the hairpin structure. The miRNA duplex is then separated and one of the strands is incorporated into the RISC while the other strand is degraded. The miC-101 candidates were processed into a 20-23 nt long mature miRNA, (FIG. 14a). The most frequently found length of guide strands was 22 nt. Guide strand refers to the sequence comprising the first RNA sequence (or substantial part thereof, that is to target the selected target RNA sequence. The passenger strand refers to the sequence comprising the second RNA sequence (or substantial part thereof). The guide and passenger strand sequences all derived from the miRNA scaffold. The length of 22 nucleotides is longer than the 21 nucleotides of the first RNA sequence that was incorporated into the miRNA scaffold design. The length of the passenger strands ranged between 19-23 nt. In most cases, Drosha cleavage sites of the mature miC-101 at 3′ end of the pre-miC-101 candidates were precise and consistent with the prediction from miRBase except for miC33_101 and miC49_101. Following cleavage by Drosha, the hairpin of the pre-miC 101 is being cleaved by Dicer. Dicer cleavage in the hairpin generated more variability for almost all the miC variants. Processing of miC2_101, miC4_101, miC32_101 and miC33_101 yielded a high frequency of guide strands with very low percentage of the passenger strand. miC46_101 processing yielded more passenger strand, while miC49_101 and miC50_101 produced a relatively equal amount of guide and passenger strand (FIG. 14a).

The processing of the miC-451 candidates did not produce passenger strands but often generated longer guide strands than the predicted 22 nt by miRbase based on the wt miRNA structure. Drosha cleavage sites at 5′ end of the mature miC-451 are precise but the trimming of the 3′ ends of the mature miC-451 by PARN varies between candidates leading to a variety of mature lengths. miC39_451, miC43_451 and miC49_451 processing generated most often mature lengths between 21-26 nt long and processing of miC38_451 and miC50_451 often resulted in mature length longer than 27 nt (FIG. 14b).

Overall, we demonstrated that expressing different C9ORF72 target sequences from miR-101 scaffold yields a differential processing of mature guide and passenger strands. Using the miC-451 no passenger strands were detected.

miC-101 and miC-451 are Active in the Nucleus
pre-miRNAs are transported from the nucleus to cytoplasm for further processing and incorporation into the RNA-induced silencing complex (RISC). However, because C9ORF72 related ALS and FTD are characterized by accumulation of the G4C2 containing transcripts in the nucleus, active mature miC in the cell nucleus is useful from a therapeutic perspective. Based on the efficacy in vitro, we selected miC2_101 and miC4_101 as the promising candidates to target only the intronic transcripts. Similarly, miC32_101, miC46_101, miC49_451 and miC50_451 were selected for a total silencing approach based on their strong silencing efficacy in vitro. Human embryonic kidney (HEK)293T were transfected with the different miC candidates, nuclear and cytoplasmic fractions were separated and expression of the mature miC2, miC4, miC32, miC46, miC49 and miC50 in nucleus and cytoplasm was evaluated. We detected mature miC in both nucleus and cytoplasmic fractions for all miC candidates but the expression levels in nucleus was consistently ˜5 fold lower compared to cytoplasm (FIGS. 15a and 15b).

Next, we evaluated the silencing efficacy of the miC candidates in nucleus and cytoplasm by measuring the endogenous levels of total C9ORF72 mRNA. miC32_101, miC46_101 and miC49_451 caused a reduction of total C9ORF72 mRNA in both nucleus and cytoplasm (FIGS. 15c and 15d). However, the silencing efficacy in the nucleus was lower compared to cytoplasm, consistent with the lower miC levels detected in the nucleus. miC2_101 and miC4_101 which targets the intronic C9ORF72 transcripts had limited to no effect on the total C9ORF72 expression. Thus, our data suggest that the mature miC-101 and miC-451 can both shuttle from the cytoplasm to the cell nucleus and can actively induce knockdown of nuclear transcripts. Reduction of C9ORF72 was observed in both the nucleus and cytoplasm suggesting that both scaffolds can be used for further development into gene therapy for ALS and FTD.

### Reduction of Nuclear RNA Foci by miC Variants in (G4C2)44 Expressing Cells

A hallmark of the RNA mediated toxicity in ALS/FTD is the formation of toxic RNA foci by the repeat containing transcripts. We generated a cell model that develops nuclear RNA foci using methods as described previously (Su et al. (2014). Neuron 83: 1043-1050; Steptoet al. (2014) Acta Neuropathol. 127: 377-389). We expressed constructs consisting of (G4C2)44 or (G4C2)3 including 150 nt 5′ and 50 nt 3′ flanking regions linked to C9ORF72 exon 2 in HEK293T cells. Nuclear RNA foci were visualized by fluorescence in situ hybridization (FISH) using a TYE563-(C4G2)3 locked nucleic acid (LNA) probe. Using a green fluorescence protein (GFP) construct the transfection efficiency was determined to be ˜95-100% (data not shown). We observed sense RNA foci at 2 days post-transfection in ˜40% of (G4C2)44 cells, but antisense RNA foci were not detected (data not shown). RNA foci were primarily present in the nucleus. Control cells expressing a shorter (G4C2)3 repeat did not accumulate RNA foci. To evaluate whether the foci were RNA specific, transfected cells were treated with RNAse or DNase. Almost all observed foci were degraded by RNAse but not DNAse, confirming that the observed foci are primarily composed of RNA.

miC4_101 and miC32_101 were evaluated for efficacy on RNA foci formation by cotransfection. Both miC candidates significantly decreased the percentage of (G4C2)44 foci-positive cells by ˜50% after 24 hours. miScr served as control and had did not reduce the amount of foci in the cells. This confirms that our miC candidates are functional in reducing RNA foci in the cell nucleus and may reduce RNA foci in human patients as well.

### Reduction of Endogenous C9ORF72 in Cells by AAV5-miC

To further investigate the silencing of C9ORF72 in context of a gene therapy approach for ALS and FTD, we selected miC32_101 as a candidate to target total C9ORF72 based on the strong efficacy and low frequency of passenger strand formation. miC46_101 was selected as a candidate as well based on silencing efficacy, but its high amount of passenger strand observed could make it less suitable to treat patients which would indicate to useC46 candidate in a miR451 scaffold instead. Nevertheless, both candidate expression cassettes were incorporated in an AAV5 vector as previously described (Miniarikova et al. Mol Ther. 2018 Apr. 4; 26(4):947-962). Increasing doses of AAV5-miC32_101 and AAV5-miC46_101 were used to transduce HEK293T cells. Expression of the mature miC32 and miC46 was verified using TaqMan. Cells transduced with AAV5-GFP served as control for the transduction efficiency. At 3 days post-transduction, AAV5-GFP transduced ˜80% of HEK293T cells. The mature guide strand expression of miC32 and miC46 was expressed at a dose-dependent manner and resulted in a dose dependent reduction of total C9ORF72 expression at a maximum of ˜40-50%. The levels of mature miC32_101 and miC46_101 produced in transduced cells correlated well with C9ORF72 silencing. Hence, these results support further proof of concept studies in animal models of C9ORF72-ALS leading towards a miRNA-based gene therapy to treat ALS and FTD.

Overall, the combined results described above indicate that miRNAs targeting C9ORF72 could be used as therapeutics to reduce the gain of toxicity caused by the G4C2 expanded repeat of C9ORF72. We demonstrated the feasibility of different targeting approaches by miC to silence the sense, antisense or both transcripts of C9ORF72. In addition, the processing of miC in the miR-101 and miR-451 was demonstrated and both scaffolds produced mature miC that were functional in the cell nucleus and cytoplasm. Silencing of C9ORF72 was also demonstrated by miC delivered by AAV5 confirming suitability of further development of a miRNA-based gene therapy for ALS and FTD.

### AAV5 is a Promising Vector to Deliver Therapeutics to the Human CNS

The main areas affected in ALS patients are motor neurons in the brain and spinal cord, whereas neurons in the frontal and temporal lobes of the brain are mainly affected in patients with FTD. About 15% of patients develop both ALS and FTD, where different types of neurons in the brain and spinal cord are affected. There are multiple supporting evidences of other cell types of the CNS such as astrocytes, microglia, and oligodendrocytes also contributing to progression of the diseases. For example, astrocytes carrying the C9ORF72 hexanucleotide expansion showed toxicity toward motor neurons, supporting their role in ALS pathogenesis. Thus, may be advantageous if delivery of therapeutics to the CNS to treat ALS and/or FTD can target a large variety of neuronal and non-neuronal cell types. We generated and characterized different human derived iPSC-neurons and astrocytes to validate the transduction of AAV5 in different cell types of the CNS (FIGS. 16-18).

Human control (ND42245) and Frontotemporal Dementia (ND42765) iPSC cells derived from fibroblast were ordered from Coriell biorepository and was cultured on Matrigel (corning)-coated 6 wells plates in mTeSR1 (STEMCELL). For embryoid body-based neural induction, iPS cells were seeded on AggreWel1800 plates and cultured in STEMdiff Neural Induction Medium (STEMCELL) for 5 days with daily medium changes. Embroid bodies were harvested and plated on 6 wells plates coated with poly-D-lysine (Sigma-Aldrich) and laminin (Sigm-Aldrich) in STEMdiff Neural Induction Medium for 7 days with daily medium changes. Rosettes were selected with rosette selection medium and plated on poly-D-lysine and laminin coated 6-wells plates in STEMdiff Neural Induction Medium for 24 hours. For differentiation into FBN, STEMdiff Neural Induction Medium was replaced for STEMdiff Neuron Differentiation medium (STEMCELL) and neuroprogenitor cells were differentiated for 5 days. For differentiation into astrocytes, neuroprogenitor cells were differentiated in STEMdiff astrocyte differentiation medium. The neuroprogenitor cells were then plated on poly-D-lysine and laminin coated plates in STEMdiff Neuron Maturation medium (STEMCELL) for one week or STEMdiff Astrocytes Maturation medium for 3 weeks. The mature FBN and astrocytes were stored in liquid nitrogen in neuroprogenitor freezing medium (STEMCELL). Cryopreserved non-diseased mature dopaminergic neurons (iCELL Dopaneurons, 01279, Cat # C1028, Lot #102477) were ordered at FUJIFILM Cellular Dynamics, Inc. Cryopreserved non-diseased mature motor neurons (cat #40HU-005, lot #400089) were ordered at iXCells Biotechnologies. AAV5 GFP was produced and characterized as described. For transductions with AAV, FBN, DPN and MN were plated in 24-wells plates at 0.3*106 cells per well. Astrocytes were plated at 0.1*10⁶ cells per well in STEMdiff Astrocyte Maturation medium (STEMCELL) on matrigel coated plates. FBN were plated in STEMdiff Neuron Maturation medium (STEMCELL) on poly-D-lysine and laminin coated plates. Dopaminergic neurons were plated in iCell Neural Base medium FUJIFILM Cellular Dynamics, Inc) according to the manufacturer description on poly-D-lysine and laminin coated plates. Motor neurons were plated in motor neuron maintenance medium according to the manufacturer description matrigel coated plates. After 1 week of acclimation, cells were transduced with AAV5 for 1-2 weeks.

Induced pluripotent stem cells were induced into neural progenitor state and differentiated into frontal brain like neurons (FBN) or astrocytes. Immunohistochemistry was performed and about 60% of FBN were ß-tubulin III positive and glial fibrillary acidic protein (GFAP) negative implicating a successful differentiation rate into mature neurons. Similarly, mature astrocytes were ˜90% GFAP positive, confirming a successful differentiation. Mature dopaminergic neurons representing neurons in the mid brain region were obtained from cellular dynamic international (CDI) and were ˜90% tyroxine hydroxylase positive. Mature motor neurons were ordered at ixcellsbiotechnolgy and ˜85% were choline acetyltransferase (CHAT) positive. Transduction with AAV5-GFP showed that ˜90% of all the different neuronal cell types were GFP positive (FIG. 16). Merge images of Immunohistochemistry for GFP combined with either ß-tubulin III, TH, GFAP or CHAT antibodies confirmed that all four cell types were transduced by AAV5 (FIG. 17). To compare the transduction rate of the different cell types, we isolated DNA and RNA of transduced cells and looked at vector copies and GFP mRNA expression in the cells (FIGS. 18a and 18b). A dose dependent and similar transduction pattern was observed in all the different cells which correlated with the GFP expression. Thus, AAV5 can efficiently transduce different human cell types of the CNS including FBN, dopaminergic neurons, motor neurons and astrocytes and is a suitable vector candidate to deliver therapeutic genes to the CNS to treat neurogenerative diseases, and in particular for targeting C9ORF72 as described herein.

C9ORF72 Levels is Reduced in Cells from FTD Patient
Induced pluripotent stem cells from a healthy control person (ND42245) and a FTD patient (ND42765) were differentiated into FBN (FTD-FBN) and astrocytes (FIG. 28a). RT-qPCR was performed 2 weeks after maturation to detect total C9ORF72 mRNA and the sense intronic transcript levels to compare the expression levels in these cells. Primers amplifying a region spanning exon 2-exon 4 were used to detect total C9ORF72 mRNA and the sense intronic transcripts were detected with primers amplifying an intron region (Liu et al. (2017) Cell Chem. Biol. 24: 141-148.). The levels of total C9ORF72 mRNA was significantly reduced in the cells derived from the FTD patient. This is similar to the results observed in the transcriptome analysis of the RNA-seq data from C9ORF72 ALS patients as compared with controls (see FIG. 2a). A reduction of ˜60% was observed in FBN and ˜25% in astrocytes from FTD compared to control cells (FIG. 19a). Interestingly, the sense intronic transcript levels were increased by ˜30% in FBN and ˜20% in astrocytes from the FTD patient compared to the control (FIG. 19b). This is similar to the results observed in the transcriptome analysis of the RNA-seq data from C9ORF72 ALS patients as compared with controls (see FIG. 3b). Thus, while total C9ORF72 mRNA levels are reduced in the FTD patient relative to controls, the repeat containing RNA transcripts in the FTD patient appear to accumulate in IPSC derived FBNs and astrocytes from the patient.
Lowering of C9ORF72 in iPSC-Neurons by AAV5-miC
miC32 (SEQ ID NO. 86) and miC46 (SEQ ID NO. 104) were designed in exon 2 and exon 11 respectively, aiming for a total silencing of C9ORF72 mRNA, targeting all sense C9ORF72 transcripts, whether or not they contain the G4C2repeat (FIG. 1). miC2 and miC4 were designed in intron 1 to selectively silence the sense G4C2 repeat containing transcripts (sense intronic transcripts). To determine whether miC delivered by AAV5 is functional in cells, FTD-FBNs were transduced with AAV5-miC2, AAV5-miC4, AAV5-miC32 and AAV5-miC46. We observed high expression levels of all four mature miC after 2 weeks, suggesting a successful transduction by AAV5-miC and efficient processing into a mature miC (FIG. 20a). Next, we determined the efficacy of the miC candidates in FBNs. The sense intronic transcript levels was reduced by ˜40% in FBNs transduced with miC2 and miC4 while the C9ORF72 mRNA levels were apparently not affected (FIG. 20b). Thus, both C2 and C4 candidates target the repeat containing transcripts and allow to preserve normal levels of the C9ORF72 mRNA. For candidates targeting the total C9ORF72 mRNA, both miC32 and miC46 reduced the levels of C9ORF72 mRNA (˜50%) and the sense intronic transcript (˜40%). Thus, the repeat containing sense transcripts is also targeted by C32 and C46.

Additionally, we investigated silencing of C9ORF72 in motor neurons, which is highly affected in ALS. Control (healthy) mature motor neurons where obtained from IXcells and acclimatized for 1 week. Total and intronic C9ORF72 was detected in non-transduced motor neurons but the intronic C9ORF72 expression was very low and slightly above the detection limit (data not shown). After acclimatization, motor neurons were transduced with AAV5-miC32 and AAV5-miC46 for 2 weeks. We found high expression of the mature miC32 and miC46 after transductions confirming that human motor neurons are transduced by AAV5 (FIG. 21a). Consistently we observed ˜40% reduction of total C9ORF72 mRNA by both miC candidates and a mild reduction of the intronic C9ORF72 (˜20%) (FIGS. 21b and 21c). Thus, AAV5-miC can transduce motor neurons and induce lowering of C9ORF72. Altogether, reduction of total and intronic C9ORF72 levels were reduced in FBNs and motor neurons, confirming that both neuronal cell types are transduced, and the miC candidates are effective in these cells.

### Targeting C9ORF72 in the Nucleus of IPSC Derived Neurons by AAV-miC

Accumulation of the G4C2 containing transcripts in the cell nucleus appears to contribute to the progression of both ALS and FTD. These transcripts form RNA foci in the cell nucleus that sequester RNA binding proteins and inhibit their function, or are transported to the cytoplasm for RAN translation into toxic DPRs. Thus, efficacy in the cell nucleus by miC may contribute in a therapeutic approach that targets i.a. RNA mediated toxicity in ALS and FTD. We now evaluated whether transduction of iPSC neurons by AAV5-miC is sufficient to express the mature miC and reduce C9ORF72 levels in nucleus.

FTD-FBNs were transduced with AAV5-miC32 and AAV5-miC46 for one week and RNA was isolated from nuclear and cytoplasmic fractions. The percentage of RNA transcript in nucleus and cytoplasm was calculated. About 80% of total C9ORF72 mRNA was detected in the nucleus and ˜20% was measured in the cytoplasm of non-transduced FBN. Whereas, the sense intronic transcripts was predominately (˜95%) expressed in nucleus of FTD-FBNs (FIG. 22a). Thus, both C9ORF72 mRNA and the sense intronic transcripts was significantly higher expressed in the nucleus of FTD-FBNs. Next, the percentage of the mature miC and the silencing of C9ORF72 was determined in nucleus and cytoplasm after transducing FTD-FBNs with AAV5-miC32 and AAV5-miC46. About 20% of the mature miC32 was detected in the nucleus while ˜80% was measured in the cytoplasm (FIG. 22b). In cells treated with AAV5-miC46, ˜90% of the mature miC was expressed in the cytoplasm and ˜10% in the nucleus. About a 30% reduction of C9ORF72 mRNA was observed in the nucleus and ˜40% reduction was detected in the cytoplasm (FIGS. 23a and 23b). Consistently, ˜25% reduction of the sense intronic transcripts was observed in the nucleus (FIG. 23c). These results suggest that the mature miC32 and miC46 can both shuttle from the cytoplasm to the cell nucleus and can actively induce a reduction of C9ORF72 mRNA and the sense intronic transcripts. Reduction of C9ORF72 was observed in both nucleus and cytoplasm suggesting that the miRNAs are capable to target target accumulation of the repeat containing transcripts in both cellular structures.

### Reduction of C9ORF72 and RNA Foci in Tg(C9ORF72_3) Line 112 Mice

Having established the efficacy of AAV5-miC in different cells, including human neuronal cell types, we next evaluated their efficacy in vivo in the Tg(C9ORF72_3) line 112 mice (O'Rourke et al. (2015) Neuron 88: 892-901). This mouse model contains several tandem copies of the human C9ORF72 with repeat sizes ranging from 100-1000 repeats. Although the progressive neurodegeneration seen in ALS and FTD patients is not recapitulated, the Tg(C9ORF72_3) line 112 mice exhibit some of the pathological features such as RNA foci (starting at ˜3 months of age) and poly GP protein (starting at ˜6-20 months of age). Three months old mice were injected bilaterally in the striatum with AAV5-GFP, AAV5-miC32 and AAV5-miC46. Mice were sacrificed 6 weeks post injection to determine the genomic copy distribution of AAV5, mature miC expression, C9ORF72 lowering and the effect on RNA foci formation. A widespread distribution of AAV5 to the cortex, striatum and midbrain was observed after injections in the striatum (FIG. 24a). A weak transduction of the cerebellum was observed while the spinal cord was not transduced. Consistent with the AAV5 distribution, small RNA TaqMan showed high expression of miC32 and miC46 in the cortex and striatum which resulted in a 20-40% lowering of C9ORF72 mRNA and the sense intronic transcripts (FIGS. 24b, 25a and 25b)). A lowering of the mice C9ORF72 ortholog (3110043021 Rik) by AAV5-miC32 and AAV5-miC46 was also detected in striatum and cortex but no behavioral and/or phenotypic changes were.

We further investigated the miC processing in the mouse model. After transcription of the miC construct, the primary miR-101 is processed by Drosha cleavage at 3′ end and then by Dicer cleavage at the hairpin structure into a miRNA duplex. The miRNA duplex is then separated, and the guide strand is usually incorporated into the RNA-induced silencing complex (RISC) while in most cases the passenger strand is degraded. The processing of the miC32 and miC46 was analyzed by next-generation sequencing (NGS) for small transcriptome to determine the ratio of guide and passenger strands that are produced. small transcriptome analysis was performed on RNA isolated from striatum of 4 mice that was injected with AAV5-miC32 or AAV5-miC46. For each sample, we obtained between 15-30 million small RNA reads that were subsequently adaptor-trimmed and aligned against the corresponding reference sequence. All reads shorter than 10 nucleotide (nt), longer than 45 nt, or represented less than 10 times were excluded from the analysis. miC32 was processed into predominantly strands comprising a first RNA sequence, an RNA sequence that has complementarity to the C9orf72 target sequence (˜87%) of 19-20 nucleotide (nt) long (also referred to as guide strand), with low percentages of the sense strand, also referred to as passenger strand (˜13%). However, miC46 processing yielded higher amounts of sense strands (“passenger strand”) (˜82%) of between 19-22 nt long and low amounts (˜18%) of RNA strands targeting C9orf72 (FIG. 26a, table S1).

RNA foci formation formed by the repeat containing transcripts is considered a hallmark of the RNA mediated toxicity in ALS/FTD. Fluorescence in situ hybridization (FISH) using a TYE563-(C4G2)3 locked nucleic acid (LNA) probe RNA FISH showed that ˜60-80% of cells in cortex, hippocampus and cerebellum of the Tg(C9ORF72_3) line 112 mice contained RNA foci (FIG. 26b) (O'Rourke et al. (2015) Neuron 88: 892-901). We evaluated RNA foci formation in this mouse model and the presence of sense and antisense RNA foci in the cortex, hippocampus and cerebellum was confirmed, whereas low amounts of RNA foci were detected in the striatum. Next, the efficacy of AAV5-miC32 and AAV5-miC46 on reduction of RNA foci was determined. Both miC candidates significantly decreased the percentage of sense (G4C2) foci-positive cells in the cortex and hippocampus (FIGS. 27a, 27b and 29). Our data confirm that AAV5 delivered miC candidates against total C9ORF72 mRNA are functional in reducing nuclear RNA foci in brain tissues of the Tg(C9ORF72_3) line 112 mice.

To conclude, we demonstrated that AAV5 can transduce different cell types of the CNS relevant for ALS/FTD treatment and that miC candidates targeting C9ORF72 are effective in a mouse model for ALS/FTD. Furthermore, we show that total C9ORF72 mRNA and sense intronic C9ORF72 transcripts can be lowered in both nucleus and cytoplasm.

### EMBODIMENTS

1. An expression cassette encoding a double stranded RNA comprising a first RNA sequence and a second RNA sequence wherein the first and second RNA sequence are substantially complementary, wherein the first RNA sequence has a sequence length of at least 19 nucleotides and is substantially complementarity to a target RNA sequence comprised in an RNA encoded by a human C9orf72 gene.
2. An expression cassette according to embodiment 1, wherein said first and second RNA sequence are comprised in a pre-miRNA scaffold, a pri-miRNA scaffold or a shRNA.
3. An expression cassette according to embodiment 1 or 2, wherein said first and second RNA sequence are comprised in a pre-miRNA scaffold or a pri-miRNA scaffold from miR101 or miR451.
4. An expression cassette according to any one of embodiments 1-3, wherein said first RNA sequence is comprised in a guide sequence.
5. An expression cassette according to any one of embodiments 1-4, wherein said first RNA sequence and said second RNA sequence when expressed in a cell are processed by the cell to produce a guide sequence comprising the first RNA sequence.
6. An expression cassette according to any one of embodiments 1-5, wherein said first RNA sequence is substantially complementary to a target RNA sequence comprised in antisense RNA transcripts encoded by the human C9orf72 gene.
7. An expression cassette according to embodiment 6, wherein said first RNA sequence is substantially complementary to SEQ ID NO. 15 or SEQ ID NO. 21.
8. An expression cassette according to embodiment 7 wherein the first RNA sequence is of SEQ ID. 68 or SEQ ID NO. 74.
9. An expression cassette according to embodiment 8 wherein the first RNA sequence and second RNA sequence are selected from the group consisting of the combinations of SEQ ID NOs. 68 and 117 or 153 and SEQ ID NOs. 74 and 118 or 154.
10. An expression cassette according to embodiment 9, wherein said encoded RNA comprises an RNA sequence selected from the group consisting of SEQ ID NOs. 119, 120, 139, 140, 155 and 156.
11. An expression cassette according to any one of embodiments 1-5, wherein said first RNA sequence is substantially complementary to exon 2 sequence comprising RNA transcripts encoded by the human C9orf72 gene.
12. An expression cassette according to embodiment 11, wherein said first RNA sequence is substantially complementary to SEQ ID NO. 32.
13. An expression cassette according to embodiment 12 wherein the first RNA sequence is SEQ ID NO. 86 or SEQ ID NO. 91.
14. An expression cassette according to embodiment 13 wherein the first RNA sequence and second RNA sequence are selected from the group consisting of the combinations of SEQ ID NOs. 86 and 121, and SEQ ID NOs. 91 and 122 or 157.
15. An expression cassette according to embodiment 14, wherein said encoded RNA comprises an RNA sequence selected from the group consisting of SEQ ID NOs. 123, 124, 141, 142 and 158.
16. An expression cassette according to any one of embodiments 1-5, wherein said first RNA sequence is substantially complementary to intron 1 sequence comprising RNA transcripts encoded by the human C9orf72 gene.
17. An expression cassette according to embodiment 16, wherein said first RNA sequence is substantially complementary to SEQ ID NO. 2, SEQ ID NO. 4 or SEQ ID NO. 31.
18. An expression cassette according to embodiment 17 wherein the first RNA sequence is selected from the group consisting of SEQ ID NO. 52, SEQ ID NO. 54, SEQ ID NO. 58, SEQ ID NO. 59 and SEQ ID NO. 85.
19. An expression cassette according to embodiment 16 wherein the first RNA sequence and second RNA sequence are selected from the group consisting of the combinations of SEQ ID NO. 52 and 125; SEQ ID NO. 58 and SEQ ID NO. 126 or 159; SEQ ID NO. 54 and SEQ ID NO. 127, SEQ ID NO. 59 and SEQ ID NO128 and 160; and, SEQ ID NO. 85 and 129.
20. An expression cassette according to embodiment 19, wherein said encoded RNA comprises an RNA sequence selected from the group consisting of SEQ ID NOs 130, 131, 132, 133, 134, 143, 144, 145, 146, 147, 161 and 162.
21. An expression cassette according to any one of embodiments 1-5, wherein said first RNA sequence is substantially complementary to exon 11 sequence comprising RNA transcripts encoded by the human C9orf72 gene.
22. An expression cassette according to embodiment 21, wherein said first RNA sequence is substantially complementary to SEQ ID NO. 46.
23. An expression cassette according to embodiment 22 wherein the first RNA sequence is SEQ ID NO. 104 or SEQ ID NO. 109.
24. An expression cassette according to embodiment 23 wherein the first RNA sequence and second RNA sequence are selected from the group consisting of the combinations of SEQ ID NOs. 104 and 135, and, SEQ ID NOs. 109 and 136 or 163.
25. An expression cassette according to embodiment 24, wherein said encoded RNA comprises an RNA sequence selected from the group consisting of SEQ ID NOs. 137, 138, 148, 149 and 164.
26. An expression cassette comprising a combination of a first and second RNA sequence as defined in any one of embodiments 6-10 and a first and second RNA sequence as defined in any one of embodiments 11-15.
27. An expression cassette comprising a combination of a first and second RNA sequence as defined in any one of 6-10 and a first and second RNA sequence as defined in any one of embodiments 16-20.
28. An expression cassette comprising a combination of a first and second RNA sequence as defined in any one of embodiments 6-10 and a first and second RNA sequence as defined in any one of embodiments 21-25.
29. An expression cassette according to any one of embodiments 1-28, wherein the expression cassette comprises a PGK promoter, a CMV promoter, a neurospecific promoter or a CBA promoter operably linked to said first RNA sequence and said second RNA sequence.
30. A gene therapy vector comprising the expression cassette according to any one of embodiments 1-29.
31. A gene therapy vector according to embodiment 30, wherein the gene therapy vector is an AAV vector.
32. A gene therapy vector according to embodiment 30, wherein the gene therapy vector is an AAV vector of serotype 5.
33. A gene therapy vector according to any one of embodiments 30-32, for use in a medical treatment.
34. A gene therapy vector according according to embodiment 33, for use in the treatment of ALS and/or FTD.
35. A gene therapy vector according to embodiment 33 or embodiment 34, wherein said first and second RNA sequences when expressed in a cell reduce expression of RNA encoded by a human C9orf72 gene both in the cell nucleus as in the cytoplasm.
36. A gene therapy vector according to any one of embodiments 33-35, wherein said gene therapy vector reduces wherein said first and second RNA sequences when expressed in a cell reduce expression of C9 RAN protein levels.
37. A gene therapy vector according to any one of embodiments 33-36, wherein said first and second RNA sequences when expressed in a cell reduce expression of G4C2 foci and/or G2C4 foci.
38. A gene therapy vector according to any one of embodiments 33-37, wherein said medical treatment comprises transfer of the vector to a motorneuron.
39. A gene therapy vector according to any one of embodiments 33-384, wherein said medical treatment comprises transfer of the vector to a human frontal brain neuron and/or anterior brain neuron.
40. A gene therapy vector according to any one of embodiments 33-39, wherein said gene therapy vector is administered to the spinal cord.
41. A gene therapy vector according to any one of embodiments 33-40, wherein said gene therapy vector is administered to the frontal lobe and/or anterior temporal lobe.
42. A gene therapy vector according to any one of embodiments 33-41, wherein said vector is administered by intraparenchymal injection.

### FIGURES

FIG. 1. A schematic of the C9ORF72 gene and expressed transcript variants. A schematic of the C9ORF72 DNA is depicted. Black boxes represent exons; exon 1a (5001-5158), exon 1b (5374-5436), exon 2 (11703-12190), exon 3 (13277-13336), exon 4 (16391-16486), exon 5 (17218-17282), exon 6 (18568-18640), exon 7 (20260-20376), exon 8 (22071-22306)Exon 9 (28160-28217), exon 10 (30201-30310), exon 11(20445-32322) on Reference Sequence: NG_031977.1. White boxes represent non-coding regions. The lines in between represent intronic sequences. The hooked arrow pointing towards the left indicates the putative RNA antisense transcription start site, which starts downstream of exon 1b. The antisense transcription termination is not exactly defined, antisense transcripts include sequences encoded by the GGGGCC repeat region. The position of the GGGGCC repeat is indicated with a triangle and is positioned in between exon 1a and exon 2b. The DNA expresses three transcript variants from the C9ORF72 DNA, V1, V2 and V3, which are depicted as well. V1 does not contain the GGGGCC repeats sequence. The three transcripts depicted schematically represent RNA molecules as expressed in the nucleus prior to splicing and export to the cytoplasm.

FIG. 2a. C9ORF72 mRNA expression by RNAseq. C9ORF72 expression was determined in the cerebellum and frontal cortex of control subjects and patients. Shown is a plot depicting the expression levels determined. Expression of Fragments per Kilobase of transcript per million mapped reads (FPKM) was plotted (Y-axis) for controls (black dots) and for patients (triangles) against the brain region analysed (X-axis, cerebellum and frontal cortex). When comparing expression levels in the cerebellum with the frontal cortex, about a 2 to 3-fold higher expression was observed in the cerebellum for both controls and patients. When comparing expression levels between controls and patients, controls had about 2-3 fold higher expression in cerebellum as patients, and controls had about 2-fold higher expression than patients in the frontal cortex.

FIG. 2b. Relative expression of predicted C9ORF72 mRNA variants in control subjects and patients. The mRNA variants in C9-ALS and controls were predicted from the mapped and aligned RNA-seq data. Isoform V1 (black bar) is predicted from reads alignment from exon 1a to exon 5 (1950 bp), isoform V2 (grey bar) from exon 1b to exon 11 (3243 bp) and isoform V3 (white bar) from exon 1a to exon 11 (3338 bp). Expression of C9ORF72 isoforms is presented in percentage of the total (100%) C9ORF72 expression (y-axis). T-tests were performed between groups and n.s. indicates no significant differences in the levels of all isoforms. In both cerebellum and cortex, the proportion of the all three transcript variants in patients and controls subject were similar.

FIG. 3a. C9ORF72 sequence conservation in patients and control subjects. The read depth is shown on the y-axis and was estimated for exon 1a, exon 1b, exon 2 and exon 11 in cerebellum (x-axis) from C9-ALS patient (white triangle) and healthy controls (black dots). The read depth is calculated by correcting the total amount of reads obtained by RNAseq per region for the area size. The higher the read depth, the better the coverage of the region by RNAseq. The read depth in exon 1a, exon 1b and intron 1 was very low in both patients and control groups. When comparing patients to controls, no significant difference was observed. Exon 2 to exon 11 were highly covered in both patients and controls. When comparing read depth between patients and controls, Exon 2 and exon 11 showed statistically significant lower coverage in patients, consistent with lower C9ORF72 mRNA expression seen in patients (FIG. 2a).

FIG. 3b. Ratio of RNAseq reads between patients and controls in cerebellum. The total amount of reads counted in different intronic and exonic regions of patients were divided by the total amount of reads from the same region of controls. This ratio is expressed on the y-axis. Exonic and intronic regions are depicted on the x-axis. If the ratio of read is lower than 1, then the total amount of reads in the patients are lower than in the controls. If the ratio of read is higher than 1, then the total amount of reads in the patients are higher than in the controls. The estimated ratio of reads of all C9ORF72 exonic regions were about 0.4. Thus, about twofold lower reads patients in patients compared to controls. Intron 1 had a ratio of about 1, thus no significant difference between patient and controls. Intron 2-4 had 1,4 times higher reads in patients compared to controls. Introns 5, 6 and 7 were excluded as these could potentially be 3′UTR of the short C9ORF72 variant. Coverage of intron 8, 9 and 10 was lower in patients.

FIG. 4a. Schematic representation of the position of the miC candidates on human C9ORF72. The positions of the miC target sites are indicated with numbers. The numbers on the top are miC candidates designed in the miR101 scaffold (miC_101). The other numbers are miC candidates designed in miR451 scaffold. miC expression constructs miC1-miC11 and miC22-miC31 were designed in intron 1 to target only the sense intronic transcripts. miC32-miC50 were designed in exon 2 or exon 11 to target all sense C9ORF72 transcripts. miC12*-miC22* were designed on the antisense strand to target only the antisense transcripts and are indicated with an asterisk (*).

FIG. 4b. Schematic of the miC-101 and miC-451 secondary structures. The scaffolds were selected from miRBase database (www.mirbase.org). miR-101 can be processed into active guide strands and in some cases passenger strands. Both strands are depicted in the precursor miC 101 structure. miR-451 produces only guide strands.

FIG. 5a. Schematic of the miC constructs. The first drawing shows the composition of the miC-451 constructs. The second drawing shows the miC-101 constructs. The third drawing shows the miC451-101 constructs. All three constructs contain the CMV early enhancer/chicken β actin (CAG) promoter, the primary miC sequence in the miR451 and/or miR101 scaffold and the human growth hormone polyadenylation (hGH polyA) signal.

FIG. 5b. Schematic of the five reporter constructs. Reporter constructs were used to screen miC candidates. To represent the C9ORF72 sense transcripts, sequences from C9ORF72 intron 1 (int 1a and int 1b), exon 2 (ex2) and exon 11 (ex11) were cloned downstream of the renilla luciferase gene. In addition, firefly luciferase was co-expressed from the vector as an internal control. For the antisense reporter (AS), the intronic antisense sequence was cloned. The miC candidates and their binding positions are shown on top of each drawing. Between brackets are the scaffold (miR101 or miR451).

FIG. 6. Graph showing silencing of intron 1 reporters by the intron 1 targeting miC variants. HEK293T cells were co-transfected in a 1:1 ratio with the luciferase reporter constructs and the different miC variants. Renilla and firefly were measured 2 days post-transfection and renilla was normalized to firefly expression. Scrambled miRNA (miScr) served as a negative control and was set at 100% (y-axis). The first black bar represents the miScr and did not show knockdown. The miC candidates in the miR101 scaffold are shown in white bars. miC candidates in miR451 scaffold are shown in grey bars. miC2_101, miC4_101 and miC31_101 in miR101 scaffold miC variants showed most silencing efficacy of 57%, 61% and 72% respectively. All three are underlined in the graph.

FIG. 7a. Graph showing silencing of exon 2 reporter by the exon 2 targeting miC variants. See description in FIG. 6. The most effective miC variants in miR101 were miC32_101 and miC33_101 with a silencing efficacy of 84% and 83% respectively. The most effective miC variants in miR451 were miC38_451, miC39_451, miC40_451 and miC43_451 with a silencing efficacy of 77%, 79%, 78% and 88% respectively.

FIG. 7b. Graph showing silencing of exon 11 reporter by the exon 11 targeting miC variants. See description in FIG. 6. The most effective miC variant in miR101 was miC46_101 with a silencing efficacy of 84%. The most effective miC variants in miR451 were miC49_451 and miC50_451, with a silencing efficacy of 82%%, 82% respectively.

FIG. 7c. Graph showing silencing of antisense reporter by the antisense targeting miC variants. See description in FIG. 6. The most effective miC variant was miC15_451* and miC21_451*, both with a silencing efficacy of 66%.

FIG. 8a. Dose dependent silencing of intron 1 reporter. 10 ng of the intron 1 reporters was co-transfected with 1, 5, 10 and 25 ng (x-axis) of miC2_101, miC4_101 and miC31_101. All three miC variants showed a dose dependent silencing.

FIG. 8b. Dose dependent silencing of exon 2 reporter. 10 ng of the exon 2 reporter was co-transfected with 1, 5, 10 and 25 ng (x-axis) of miC32_101, miC33_101 and miC38_451, miC39_451, miC40_451 and miC43_451. All miC variants showed a dose dependent silencing. miC32_101 had the strongest silencing efficacy.

FIG. 9a. Dose dependent silencing of exon 11 reporter. 10 ng of the exon 11 reporter was co-transfected with 1, 5, 10 and 25 ng (x-axis) of miC46_101, miC49_451 and miC50_451. All miC variants showed a dose dependent silencing. miC46_101 had the strongest silencing efficacy.

FIG. 9b. Dose dependent silencing of the antisense reporter. 10 ng of the antisense reporter was co-transfected with 1, 5, 10 and 25 ng (x-axis) of miC15_451* and miC21_451. Both miC variants showed an equal dose dependent silencing.

FIG. 10a. Dose dependent silencing of the intron 1 reporter by miC15*+31. HEK293T cells were co-transfected with 10 ng Luc-intron 1b reporter and 1, 5, 10 or 25 ng of miC15*+31 construct. miC15*+31 expresses both miC15_451* and miC31_101 to simultaneously target both sense and antisense C9ORF72 transcripts. miC31_101 was used as positive control and showed a dose dependent silencing of intron 1 reporter (white dots). miC15_451* served as negative control and showed no silencing of the intron 1 reporter (white triangles). A dose depended silencing of the intron 1 reporter was observed by miC15*+31 (black squares).

FIG. 10b. Dose dependent silencing of the antisense reporter by miC15*+31. HEK293T cells were co-transfected with 10 ng of the antisense reporter and 1, 5, 10 or 25 ng of miC15*+31 construct. miC15_451* was used as positive control and showed a dose depended silencing of the antisense reporter (white triangles). miC31_101 served as negative control and showed no silencing of the antisense reporter (white dots). A dose depended silencing of the antisense reporter was observed by miC15*+31 (black squares).

FIG. 11a. Dose dependent silencing of the intron 1 reporter by miC21*+31. HEK293T cells were co-transfected with 10 ng Luc-intron 1b reporter and 1, 5, 10 or 25 ng of miC21*+31 construct. miC21*+31 expresses both miC21_451* and miC31_101 to simultaneously target both sense and antisense C9ORF72 transcripts. miC31_101 was used as positive control and showed a dose dependent silencing of intron 1 reporter (white dots). miC21_451* served as negative control and showed no silencing of the intron 1 reporter (white triangles). A dose depended silencing of the intron 1 reporter was observed by miC21*+31 (black squares).

FIG. 11b. Dose dependent silencing of the antisense reporter by miC21*+31. HEK293T cells were co-transfected with 10 ng of the antisense reporter and 1, 5, 10 or 25 ng of miC21*+31 construct. miC21_451* was used as positive control and showed a dose depended silencing of the antisense reporter (white triangles). miC31_101 served as negative control and showed no silencing of the antisense reporter (white dots). A dose depended silencing of the antisense reporter was observed by miC21*+31 (black squares).

FIG. 12a. Expression of total C9ORF72 mRNA and sense intronic transcripts in HEK293T cells. mRNA input levels were normalized to GAPDH and set relative to total C9ORF72 (y-axis). The expression of total C9ORF72 mRNA was about 10-fold higher compared to the sense intronic transcripts in HEK293T cells (x-axis).

FIG. 12b. Endogenous total C9ORF72 mRNA lowering in transfected HEK293T cells. RT-qPCR for total C9ORF72 mRNA was performed on RNA from HEK293T cells that were transfected with 250 ng of different miC plasmids (x-axis). mRNA input levels were normalized to GAPDH mRNA. miScr served as a negative control and was set at 100% (y-axis). White bars show miC candidates in miR101 scaffold. Grey bars show miC candidates in miR451 scaffold. striped bars show the concatenated miC candidates targeting both sense and antisense C9ORF72. All the candidates showed silencing of total C9ORF72 mRNA. The strongest silencing efficacy was observed by miC31_101, miC32_101, miC33_101, and miC46_101.

FIG. 13. Endogenous knockdown of the sense intronic transcripts in transfected HEK293T cells. RT-qPCR for the sense intronic transcripts was performed on RNA from HEK293T cells that were transfected with 250 ng of different miC plasmids (x-axis). mRNA input levels were normalized to GAPDH mRNA. miScr served as a negative control and was set at 100% (y-axis). White bars show miC candidates in miR101 scaffold. Grey bars show miC candidates in miR451 scaffold. striped bars show the concatenated miC candidates targeting both sense and antisense C9ORF72. The strongest silencing efficacy was observed by miC31_101. All other miC candidates except miC38_451, miC39_451 and miC40_451 also showed a silencing of the sense intronic transcripts.

FIG. 14a. Processing of miC-101 candidates. Small RNA NGS was performed on HEK293T cells were transfected 7 lead candidates in miR101 scaffold to determine the length and ratio of guide and passenger strands. The miC-101 candidates were mostly processed into a 20-23 nt long mature miRNA. The length of the passenger strands ranged between 19-23 nt. The percentage of reads are shown on the y-axis and the miC-101 candidates on the x-axis. Processing of miC2_101, miC4_101, miC32_101 and miC33_101 yielded a high frequency of guide strands (black and grey bars) with very low percentage of the passenger strands (white bars). miC46_101 processing yielded more passenger strand, while miC49_101 and miC50_101 produced a relatively equal amount of guide and passenger strands.

FIG. 14b. Processing of miC-451 candidates. The processing of the miC-451 candidates did not produce passenger strands but often generated longer guide strands than the predicted 22 nt obtained from miRbase miC39_451, miC43_451 and miC49_451 processing generated most often mature lengths between 21-26 nt long (black, grey and striped bars) and processing of miC38_451 and miC50_451 often resulted in mature length longer than 27 nt (white bars).

FIG. 15a. Cytoplasmic and nuclear expression of miC candidates in miR_101 scaffold. nuclear and cytoplasmic fractions were separated from HEK293T cells transfected with miC2_101, miC4_101, miC32_101, miC46_101 and miC49_101 (x-axis). The expression of the mature miC in nucleus and cytoplasm was evaluated (y-axis). Mature miC was detected in both nucleus (white bars) and cytoplasm (black bars) for all miC candidates but the expression levels in nucleus was consistently ˜5 fold lower compared to cytoplasm

FIG. 15b. Expression of miC candidates in miR-451 in cytoplasm and nucleus. nuclear and cytoplasmic fractions were separated from HEK293T cells transfected with miC49_451 and miC50_451 (x-axis) and the expression was determined (y-axis). Mature miC was detected in both nucleus (white bars) and cytoplasm (black bars) for both miC candidates. The expression levels in nucleus was ˜5 fold lower compared to cytoplasm.

FIG. 15c. Reduction of total C9ORF72 mRNA by miC-101 in nucleus and cytoplasm of HEK293T cells. C9ORF72 mRNA input levels were corrected for GAPDH and BL was set at 100% (y-axis). For cells transfected with miC2_101 miC4_101 (x-axis), a mild reduction of total C9ORF72 mRNA was observed in the nucleus and cytoplasm. A stronger reduction was observed in nucleus and cytoplasm of cells transfected with miC32_101 andmiC46_101. The efficacy was consistently stronger in the cytoplasm.

FIG. 15d. Reduction of total C9ORF72 mRNA by miC-451 in nucleus and cytoplasm of HEK293T cells. For cell transfected with miC49_101, reduction of total C9ORF72 mRNA was observed in both the nucleus and cytoplasm (x-axis). The efficacy was stronger in the cytoplasm. For cells transfected with miC50_451, a mild reduction was observed only in the cytoplasm.

FIG. 16. Transduction of different iPSC-derived cells by AAV5. Human iPSCs were differentiated into mature frontal brain-like neurons (FBN), dopaminergic neurons (DPN), astrocytes (Astr) and motor neurons (MN). All cells were GFP positive at 2 weeks post transduction with AAV5-CAG-GFP.

FIG. 17. Characterization of transduced iPSC-derived cells by AAV5. Transduced mature frontal brain-like neurons (FBN) was stained positive with Anti-beta III Tubulin (B tub III) antibody. Mature dopaminergic neurons (DPN) was stained positive with anti-tyrosine hydroxylase (TH) antibody. Mature astrocytes (Astr) was stained positive with anti-glial fibrillary acidic protein (GFAP) antibody. Mature motor neurons (MN) was stained positive with anti-choline acetyltransferase (ChAT) antibody.

FIG. 18a. Transduction efficiency of AAV5 in iPSC-derived neurons. Mature frontal brain-like neurons (FBN), dopaminergic neurons (DPN), astrocytes (Astr) and motor neurons (MN) was transduced with 4,5e11, 5,4e12 and 5,4e13 genomic copies (GC) of AAV5-CAG-GFP. The transduction efficiency was determined by quantification of the amount of GC of AAV5 in the transduced cells at 2 weeks post transduction. A similar dose depended transduction pattern was observed in all four cell types.

FIG. 18b. GFP mRNA expression in transduced iPSC-derived neurons. Mature frontal brain-like neurons (FBN), dopaminergic neurons (DPN), astrocytes (Astr) and motor neurons (MN) was transduced with 4,5e11, 5,4e12 and 5,4e13 genomic copies (GC) of AAV5-CAG-GFP (x-axis). The GFP mRNA expression was determined at 2 weeks post transduction. GFP mRNA input was corrected to GAPDH and set relative to PBS (y-axis). A similar dose depended GFP expression was observed in all four cell types.

FIG. 19a. total C9ORF72 mRNA expression in frontal brain like neurons (FBN) and Astrocytes (Astr) from FTD patient and healthy subject. RNA was isolated from cells after 2 weeks of maturation to detect the endogenous expressed total C9ORF72 mRNA. RNA input levels were corrected to GAPDH and calculated relative to cell line with the highest expression of C9ORF72 (Control-FBN). The levels are shown on the y-axis. Total C9ORF72 mRNA was ˜50% lower expressed in FBN and Astr from FTD patient (white bars) compared to the healthy control cells (black bar). Total C9ORF72 mRNA expression was higher in FBN compared to Astr.

FIG. 19b. Expression of sense intronic transcripts in frontal brain like neurons and Astrocytes from FTD patient and healthy subject. RNA was isolated from cells after 2 weeks of maturation to detect the sense intronic transcripts. RNA input levels were corrected to GAPDH and calculated relative to cell line with the highest expression of C9ORF72 (FTD-FBN). The RNA levels are expressed on the y-axis. The sense intronic transcripts was ˜20% higher expressed in cells from the FTD patient (white bars) compared to the healthy control (black bar). The sense intronic transcript levels was higher in FBN compared to Astr.

FIG. 20a. Expression of mature miC guide in frontal brain like neurons after transduction with AAV5-miC. Mature frotal brain like neurons from FTD patient (FTD FBN) was transduced with 2e12 gc of AAV5-miC2, AAV5-miC4, AAV5-miC32 and AAV5-miC46 (x-axis). Cells treated with the formulation buffer (mock) or AAV5-GFP served as controls. RNA was isolated 7 days post-transduction and expression of the mature miC2, miC4, miC32 and miC46 was determined. MicroRNA input levels was normalized to U6 small nuclear RNA and set relative to cells treated with AAV5-GFP (y-axis). High expression of all four mature miC variants was detected.

FIG. 20b. Silencing of total and intronic C9ORF72 in FTD frontal brain like neurons. Mature FTD FBN were transduced with 2e12 gc of AAV5-miC2, AAV5-miC4, AAV5-miC32 and AAV5-miC46 (x-axis). RNA was isolated 7 days post-transduction and total and intronic C9ORF72 was determined. mRNA input was normalized to GAPDH and set relative to cells treated with AAV5-GFP (y-axis). The sense intronic transcripts (white bars) levels was reduced by ˜40% in FBNs transduced with miC2 and miC4 while the total C9ORF72 mRNA (black bars) levels were not affected. For candidates targeting total C9ORF72 mRNA, both miC32 and miC46 reduced the levels from both total C9ORF72 mRNA (˜50%) and the sense intronic transcript (˜40%).

FIG. 21a. miC32 and miC46 expression in transduced motor neurons. Healthy motor neurons were transduced with AAV5-GFP, AAV5-miC32 and AAV5-miC46 for two weeks. Total RNA was isolated to detect the mature miC32 and miC46. High expression of the mature miC32 and miC46 was observed in the motor neurons.

FIG. 21b. C9ORF72 reduction in motor neurons by AAV5-miC. RNA was isolated from transduced motor neurons two weeks post transduction to detect total C9ORF72 mRNA. About 40% reduction of total C9ORF72 was observed by both miC32 and miC46.

FIG. 21c. Reduction of sense intronic transcripts in motor neurons by AAV5-miC. RNA was isolated from transduced motor neurons two weeks post transduction and RT-qPCR was performed to detect the sense intronic transcripts. About 20% reduction of the sense intronic transcripts was observed motor neurons treated with both miC32 and miC46.

FIG. 22a. Nuclear and cytoplasmic expression of total C9ORF72 mRNA and sense intronic transcripts in frontal brain like neurons of FTD patient. RNA was isolated from nuclear and cytoplasmic fractions of mature frontal brain like neurons from FTD patient (FTD-FBN) to detect total C9ORF72 mRNA and sense intronic transcripts. Total C9ORF72 mRNA and sense intronic transcripts was normalized to GAPDH. The sum of nuclear and cytoplasmic C9ORF72 expression values were set at 100% (y-axis). About 83% of the total C9ORF72 mRNA was observed in the nucleus (white bar) and ˜17% in the cytoplasm (white bar). For the sense intronic transcripts, 99% was detected in the nucleus and 1% in the cytoplasm.

FIG. 22b. miC expression in nucleus and nucleus and cytoplasm of FBN of FTD patient. FTD FBN were transduced with AAV5-GFP, AAV5-miC31 and AAV5-miC46 for 7 days. RNA was isolated from nucleus and cytoplasm and expression of mature miC31 and miC46 was determined. mRNA input was normalized to GAPDH. The sum of nuclear and cytoplasmic miC expression values was set at 100% (y-axis). For miC32, about 76% of the mature miC32 was found in the cytoplasm (black bar) and about 24% in the nucleus (white bar). For miC46, about 87% of the mature miC46 was detected in the cytoplasm and ˜13% in the nucleus.

FIG. 23a. Silencing of total C9ORF72 mRNA in nucleus of frontal brain like neurons (FBN) of FTD patient. RNA was isolated from nucleus of mature FTD FBN transduced with the formulation buffer (mock), AAV5-GFP, AAV5-miC32 and AAV5-miC46 (x-axis). Total C9ORF72 mRNA was normalized to GAPDH and set relative to GFP (y-axis). Total C9ORF72 mRNA was reduced by ˜30% in the nucleus by both miC candidates.

FIG. 23b. Silencing of total C9ORF72 mRNA in cytoplasm of FBN of FTD patient. RNA was isolated from cytoplasm of mature FTD FBN transduced with the formulation buffer (mock), AAV5-GFP, AAV5-miC32 and AAV5-miC46 (x-axis). Total C9ORF72 mRNA was normalized to GAPDH and set relative to GFP (y-axis). Total C9ORF72 mRNA was reduced by ˜40% in the cytoplasm by both miC candidates.

FIG. 23c. Silencing of sense intronic transcripts in nucleus of FBN of FTD patient. RNA was isolated from nucleus of mature FTD FBN transduced with the formulation buffer (mock), AAV5-GFP, AAV5-miC32 and AAV5-miC46 (x-axis). The sense intronic transcripts was normalized to GAPDH and set relative to GFP (y-axis). A reduction by ˜30% in the nucleus by both miC candidates.

FIG. 24a. Vector copy distribution of AAV5 in mice upon intrastriatal injection. Three months old Tg(C9ORF72_3) line 112 mice were injected with 5e10gc of AAV5-GFP (black dots), 5e10gc of AAV5-miC32 (black triangles) and 1e10gc AAV5-miC46 (black squares) bilaterally in the striatum. All mice were sacrificed 6 weeks after surgeries and frontal cortex, striatum, mid brain, cerebellum and spinal cord were collected (x-axis) to determine the vector copy distribution (y-axis). The highest copies of AAV5 was detected in the cortex and striatum, followed by midbrain. Low vector copies were detected in the cerebellum, and no vector copies was detected in the spinal cord.

FIG. 24b. Expression of mature miC32 an miC46 in cortex and striatum of transduced Tg(C9ORF72_3) line 112 mice. Total RNA was isolated from the cortex and striatum (x-axis) for small RNA taqman. MicroRNA input levels was normalized to U6 small nuclear RNA and set relative to AAV-GFP mice (y-axis). High expression of mature miC was detected in both cortex and striatum by both miC candidates.

FIG. 25a. Silencing of total C9ORF72 mRNA in striatum and cortex of Tg(C9ORF72_3) line 112 mice. Total C9ORF72 mRNA was normalized to GAPDH and set relative to GFP (y-axis). Total C9ORF72 mRNA was reduced by ˜40% in cortex by both miC32 (black triangles) and miC46 (black squares). A reduction of ˜10-30% was observed in the striatum by both miC candidates.

FIG. 25b. Silencing of sense intronic transcripts in striatum and cortex of Tg(C9ORF72_3) line 112 mice. The sense intronic transcripts was normalized to GAPDH and set relative to GFP (y-axis). A reduction of ˜40% was observed in the cortex by both miC32 and miC46. About 30% reduction was observed in the striatum by both miC candidates.

FIG. 26a. Processing of miC32 and miC46 in mice. small RNA NGS was performed on striatum of transduced mice to determine the ratio of guide and passenger strands (y-axis). miC32 (x-axis) was processed into 87% of guide strand (black bar) and 13% of passenger strand (white bar). miC46 (x-axis) was processed into about 20% of guide strand and 80% of passenger strand.

FIG. 26b. detection of RNA foci in cortex of Tg(C9ORF72_3) line 112 mice. Mice brains were frozen in OCT. Slides were fixed and RNA FISH was performed using using a TYE563-(CCCCGG)3 LNA probe. RNA foci were observed in cortex of Tg(C9ORF72_3) line 112 mice (C9+) but not in WT mice (C9−). RNA foci were mainly in the nucleus and are depicted as white spots.

FIG. 27a. Reduction of RNA foci in frontal cortex. RNA foci FISH was performed on brain sections from Tg(C9ORF72_3) line 112 mice treated with AAV5-miC32 and AAV5-miC46. A reduction of RNA foci was observed in cortex of mice treated with both AAV5-miC32 and AAV5-miC46 compared to the mice treated with the formulation buffer (mock).

FIG. 27b. Quantification of RNA foci in frontal cortex of mice. Cells with 0, 1-5 or >5 foci were counted and the percentage of cells containing 0, 1-5 or more than 5 foci was calculated from 6 different images per treatment group (N=3).

About 20% of cells in the cortex had 0 RNA foci (white bar) per cell in the Tg(C9ORF72_3) line 112 mice treated with mock. After treatment with AAV5-miC32 and AAV5-miC46, this was increased to ˜50% of cells with no RNA foci. About 23% of Cells had 1-5 RNA foci (grey bar) in mice treated with mock. After treatment with AAV5-miC32 and AAV5-miC46, about 35% had 1-5 RNA foci. About 60% of Cells had more that 5 RNA foci (black bar) in mice treated with mock. After treatment with AAV5-miC32 and AAV5-miC46, this was decreased to ˜10-15%.

FIG. 28a. Differentiation of iPSC cells into frontal brain like neurons and astrocytes. iPSC cells were seeded on AggreWell800 plates and cultured in STEMdiff Neural Induction Medium until day 5 to induce embryoid bodies (EBs)formation. Embryoid bodies were harvested and replated in STEMdiff Neural Induction Medium for 7 days. At day 12, Rosettes were selected with rosette selection medium and differentiated in STEMdiff Neuron Differentiation medium or STEMdiff astrocyte Differentiation medium (STEMCELL) for 5 days. The cells were then maturated into mature frontal brain like neurons (FBN) or astrocytes (Astr) for several weeks.

FIG. 28b. Characterization of FBN. FBN were stained with Anti-beta III Tubulin (B tub III) and about 60% of cells were positive.

FIG. 28c. Characterization of astrocyte. Astrocytes were stained with anti Glial fibrillary acidic protein (GFAP) and about 90% of cells were positive.

FIG. 29. Reduction of RNA foci in hippocampus. RNA foci FISH was performed on brain sections from Tg(C9ORF72_3) line 112 mice after AAV5 treatment. A reduction of RNA foci was observed in hippocampus of mice treated with AAV5-miC32 and AAV5-miC46 compared to the mice treated with the formulation buffer (mock).

FIG. 30. DNA sequence of an expression cassette (SEQ ID NO.150) encoding a miR101 scaffold comprising a first RNA sequence of 21 nucleotides targeting C32 (SEQ ID NO.86). The expression cassette consists of a CAG promotor shown in bold (position 43-1712), encoding the second RNA sequence, shown underlined (encoding SEQ ID NO. 121, position 2025-2046), encoding the loop sequence, depicted in italics underlined, encoding the first RNA sequence shown in bold underlined (encoding SEQ ID NO. 86, position 2061-2081), the hGH poly A signal as encoded shown in greyscale (position 2329-2425). The pri-miRNA encoding sequence is shown in between brackets (position 2015-2089) (encoding pri-miRNA sequence SEQ ID NO 141). The encoded pri-miRNA sequence may be replaced e.g. by sequence encoding a sequence listed in table 16 comprising a pre-miRNA sequence as depicted in FIG. 33. The pre-miRNA sequence is underlined (position 2025-2081) (encoding SEQ ID NO. 123). The encoded pre-miRNA sequence may be replaced by a pre-miRNA encoding sequence, such as depicted in FIG. 33 and listed in tables 4, 7, 10 or 13. The first RNA sequence can be any sequence of 21 nucleotides selected to be complementary to a target sequence in the C9ORF72 gene (table 15). The second RNA sequence is selected and adapted to be complementary to the first RNA sequence. The secondary structure is checked on mfold by folding the RNA sequence using standard settings utilizing the RNA folding form, with folding temperature fixed at 37 degrees Celcius (as available online <URL:http://unafold.rna.albany.edu/?q=mfold>; Zuker et al., Nucleic Acids Res. 31 (13), 3406-15, (2003)) for folding, and adapted if necessary, into a miR-101 pri-miRNA structure as depicted in FIG. 4b.

FIG. 31. DNA sequence of an expression construct (SEQ ID NO. 151) encoding a miR451 scaffold comprising a first RNA sequence of 22 nucleotides targeting C32 (SEQ ID NO.91). The expression cassette comprises a CAG promotor shown in bold (position 43-1712), the sequence encoding the first RNA sequence shown in bold and underlined (position 2031-2052, encoding SEQ ID NO. 91), the second RNA sequence is shown underlined (position 2053-2070, encoding SEQ ID NO. 122), the hGH poly A signal shown in bold (2318-2414). The pri-miRNA sequence is shown between brackets (position 2015-2086, encoding SEQ ID NO. 142). The pri-miRNA sequence may be replaced e.g. by a sequence listed in table 16 encoding a pre-miRNA encoding sequence as depicted in FIG. 34. The pre-miRNA sequence comprises the first RNA sequence and the second RNA sequence and is shown underlined (position 2031-2070) (encoding SEQ ID NO. 124). The encoded pre-miRNA sequence may be replaced by another pre-miRNA encoding sequence, such as depicted in FIG. 34 and listed in tables 4, 7, 10 or 13. Such pre-miRNA sequence may be comprised e.g. in a pri-miRNA sequence as listed in table 16. The first RNA sequence can be any sequence of 22 nucleotides selected to bind and target a sequence in the C9ORF72 gene (table 15). The second RNA sequence is selected and adapted to be complementary to the first RNA sequence. The secondary structure is checked on mfold by folding the RNA sequence using standard settings utilizing the RNA folding form, with folding temperature fixed at 37 degrees Celcius (as available online <URL:http://unafold.rna.albany.edu/?q=mfold>; Zuker et al., Nucleic Acids Res. 31 (13), 3406-15, (2003)) for folding, and adapted if necessary, into a miR-451 pri-miRNA structure as depicted in FIG. 4b.

FIG. 32. DNA sequence of an expression construct (SEQ ID NO. 152) encoding a single RNA transcript with a miR451 scaffold comprising a first RNA sequence targeting C15 (SEQ ID NO.68) and a miR101 scaffold comprising a first RNA sequence targeting C31 (SEQ ID NO. 85). The construct consists of a CAG promotor shown in bold (position 43-1712), encoding a pri-miRNA sequence in a miR451 scaffold targeting C15 depicted between single brackets (position 1969-2040, encoding SEQ ID NO. 119). The pri-miRNA sequence consists of the sequence encoding the first RNA sequence targeting C15 shown in bold and underlined (position 1985-2006, encoding SEQ ID NO.139) and the second RNA sequence shown underlined (position 2007-2024, encoding SEQ ID NO.117). The pre-miRNA sequence consists of the first RNA sequence and the second RNA sequence and is shown underlined (position 1985-2024, encoding SEQ ID NO. 119). The DNA sequenc encoding a pri-miRNA sequence in a miR101 scaffold targeting C31 is shown between the double brackets (position 2457-2531, encoding SEQ ID NO. 147). The sequence encoding the second RNA sequence is shown in underlined (position 2467-2488, encoding SEQ ID NO.129) and the sequence encoding the first RNA sequence shown in bold and underlined (position 2503-2523, encoding SED ID NO. 85). The loop sequence is shown in italics and underlined. The pre-miRNA sequence is shown underlined (position 2467-2523, encoding SEQ ID NO. 134). The hGH poly A signal shown in bold (position 2775-2871. As described above for FIGS. 30 and 31, likewise, DNA sequences encoding first RNA sequences, second RNA sequences, pre-miRNA sequences and/or pri-miRNA may be replaced with corresponding sequences that target C9ORF72 sequences other than C15 and/or C31 as described herein.

FIG. 33. Predicted RNA structures of selected pri-miRNA sequences in miR101. Sequences of the secondary RNA sequences depicted are listed in Table 16. Structures were made using M-fold using standard settings, utilizing the RNA folding form, with folding temperature fixed at 37 degrees Celcius (as available online <URL:http://unafold.rna.albany.edu/?q=mfold>; Zuker et al., Nucleic Acids Res. 31 (13), 3406-15, (2003).

FIG. 34. Predicted RNA structures of selected pri-miRNA sequences in miR451. Sequences of the secondary RNA sequences depicted are listed in Table 16. Structures were made using M-fold using standard settings, utilizing the RNA folding form, with folding temperature fixed at 37 degrees Celcius (as available online <URL:http://unafold.rna.albany.edu/?q=mfold>; Zuker et al., Nucleic Acids Res. 31 (13), 3406-15, (2003).

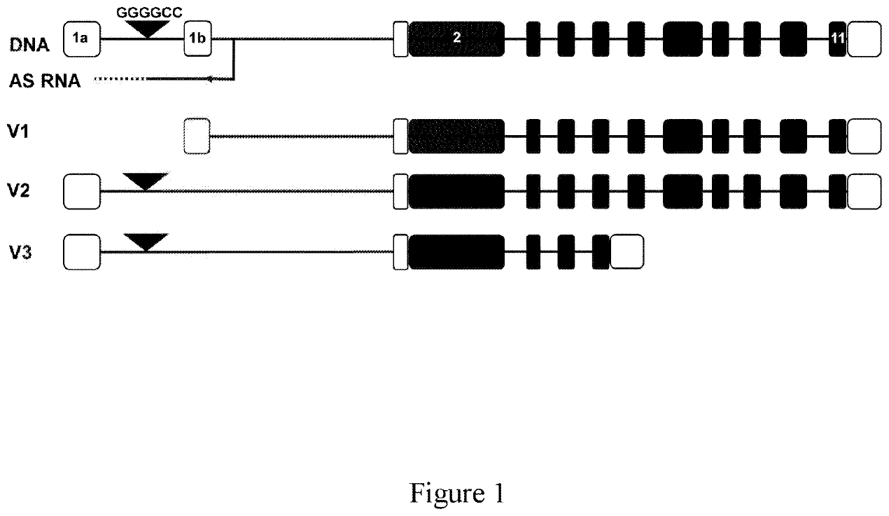

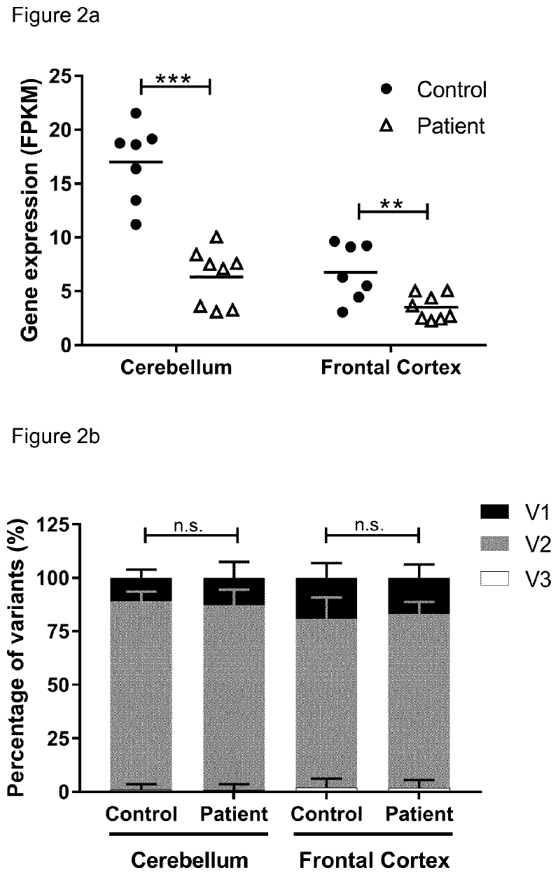

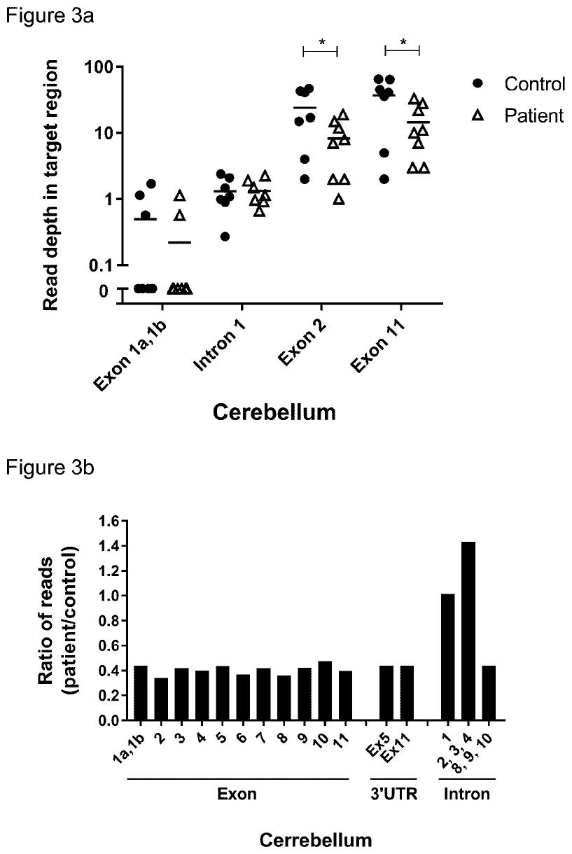

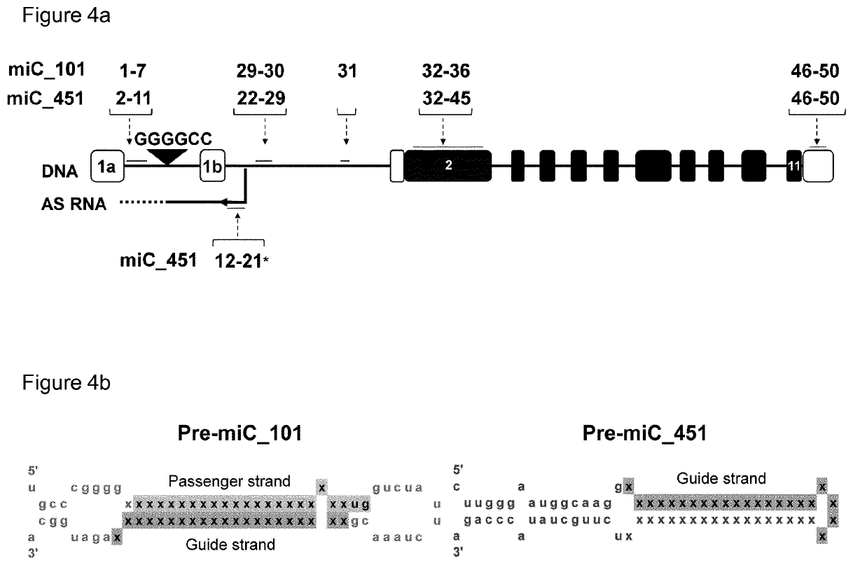

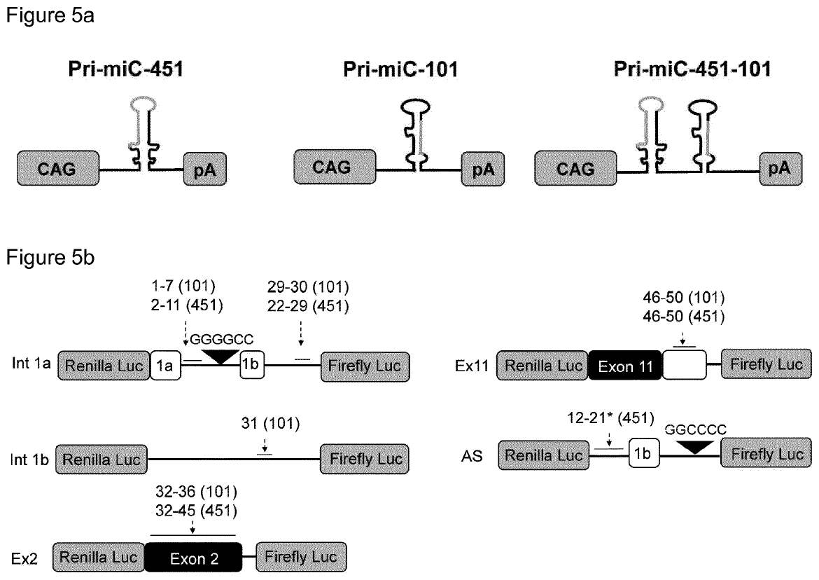

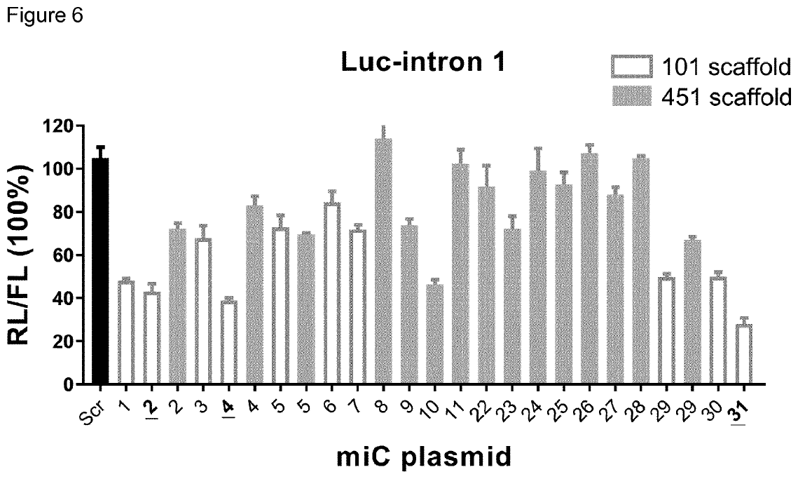

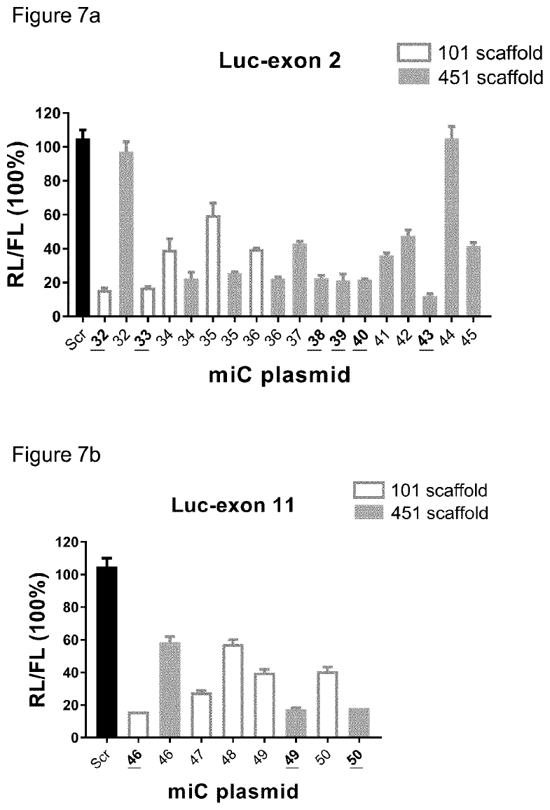

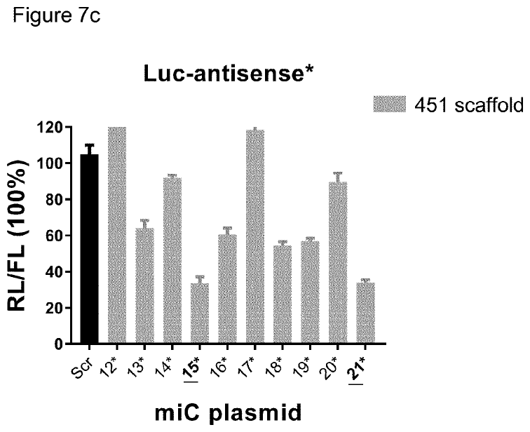

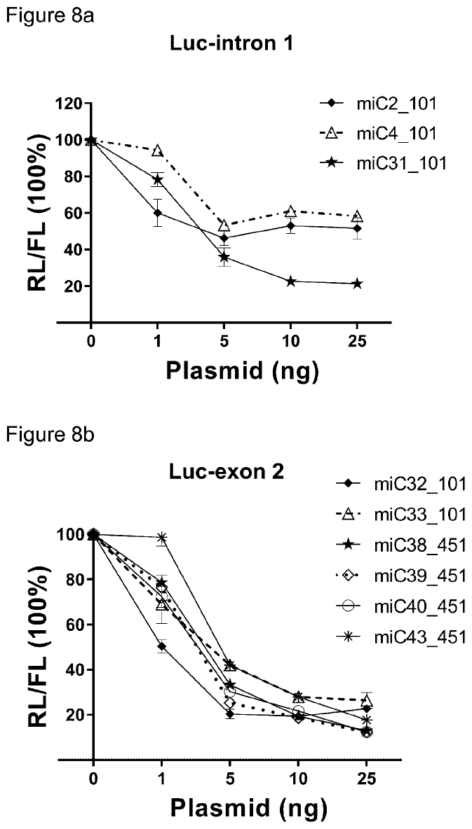

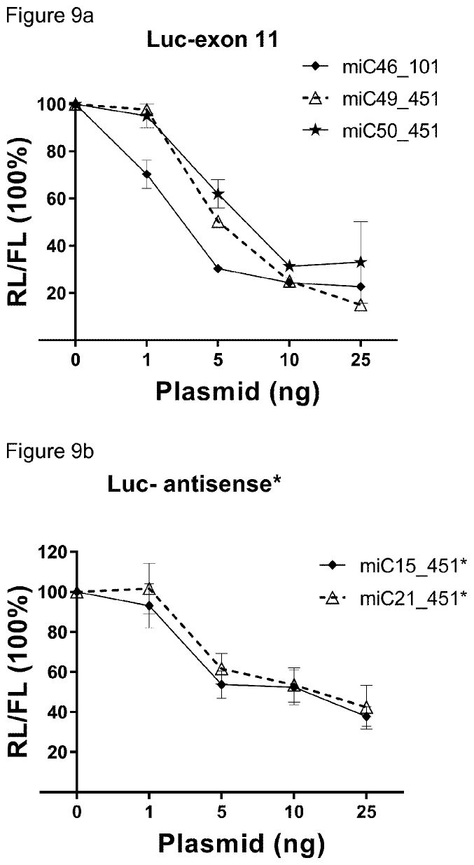

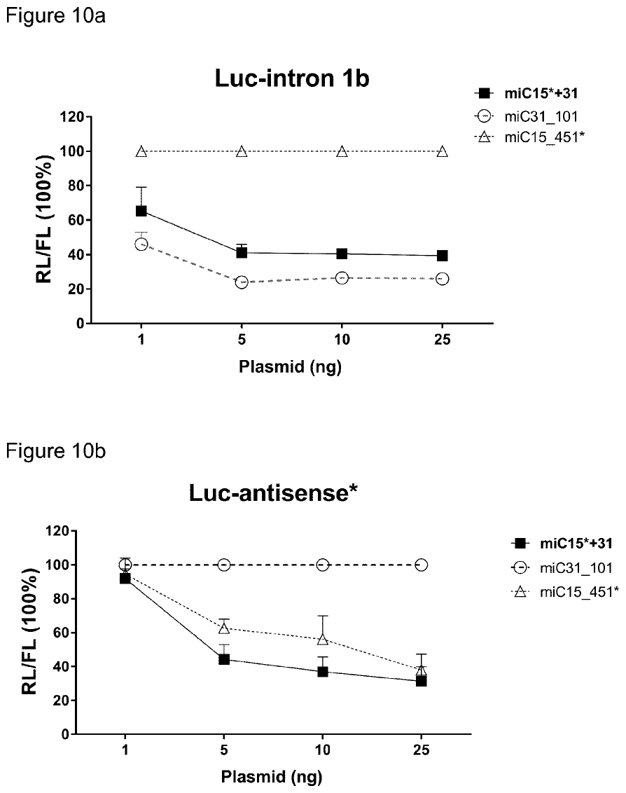

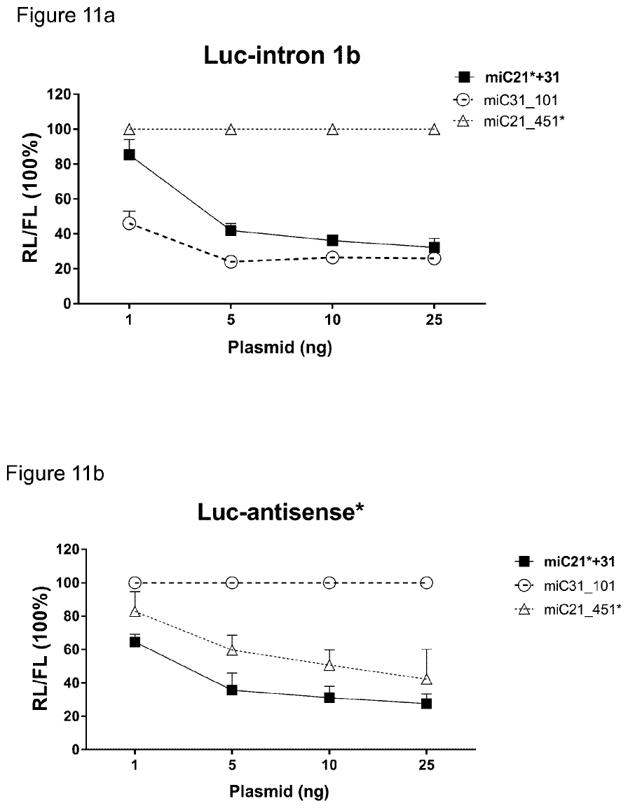

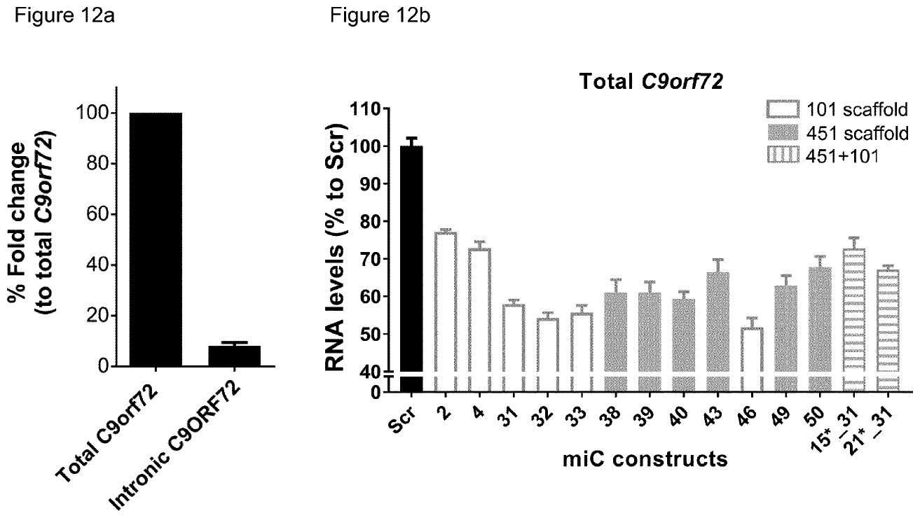

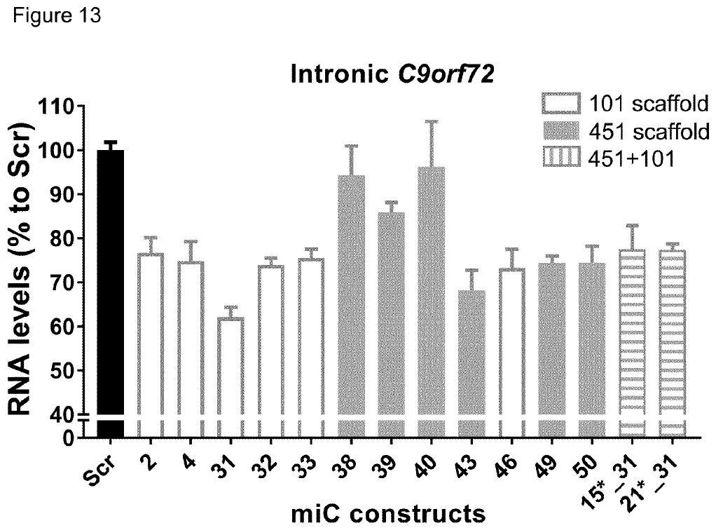

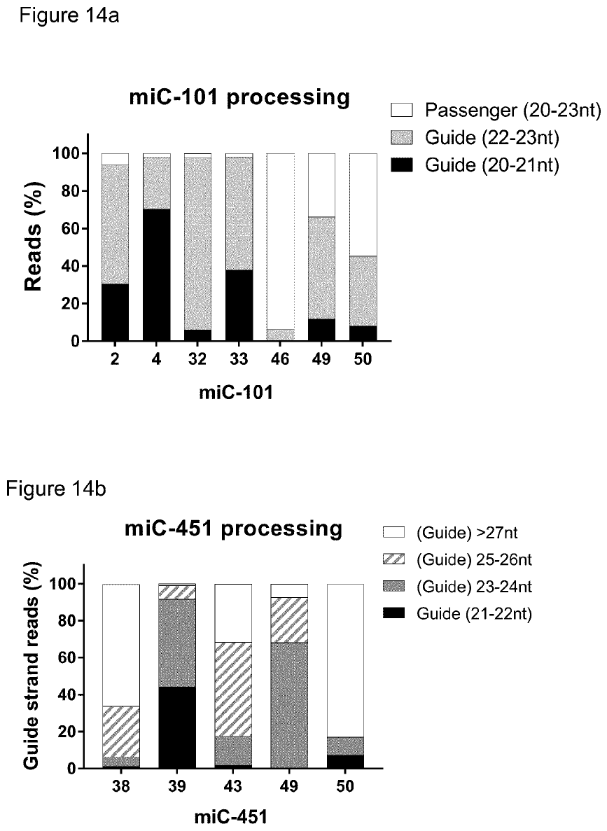

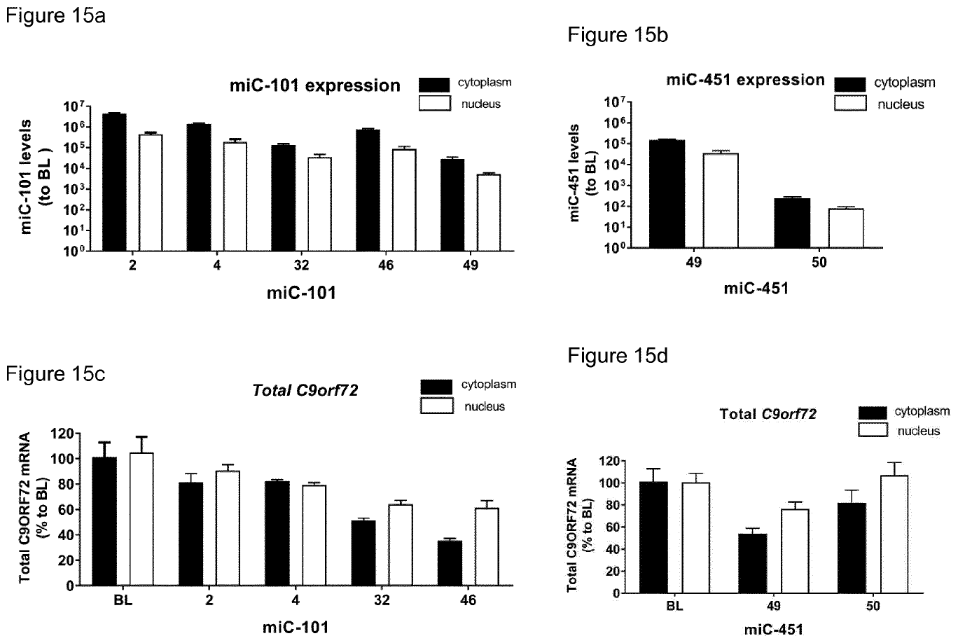

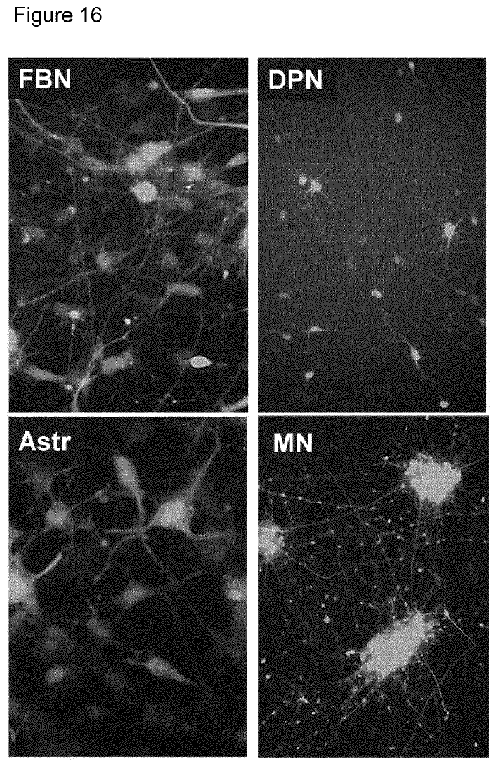

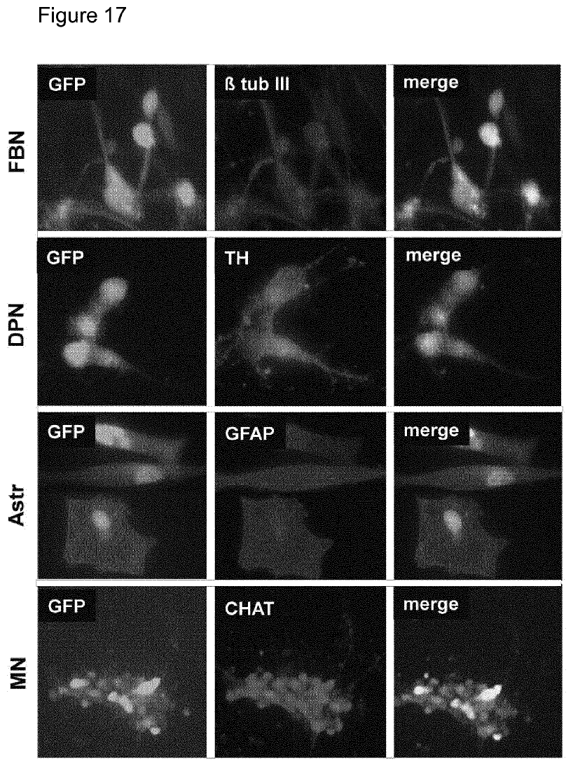

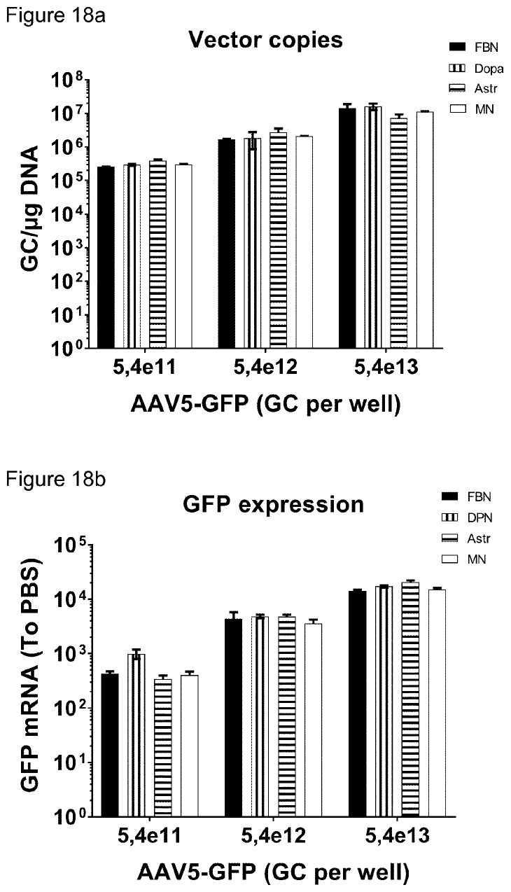

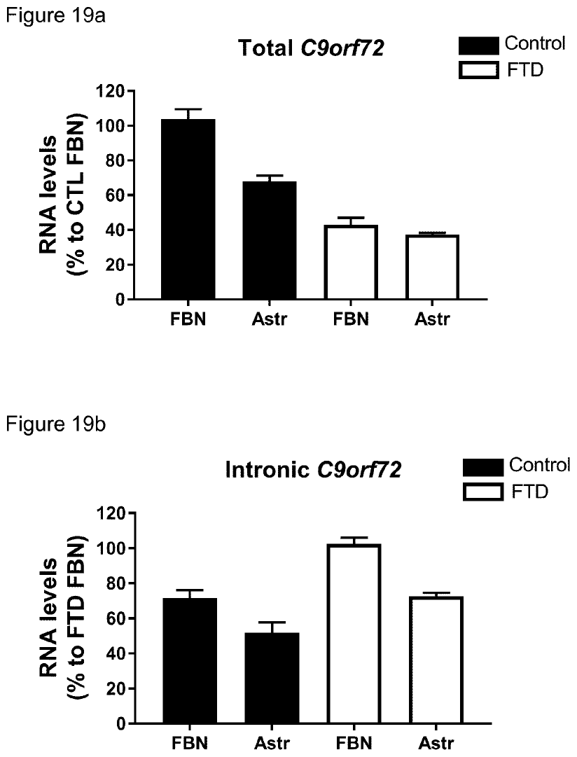

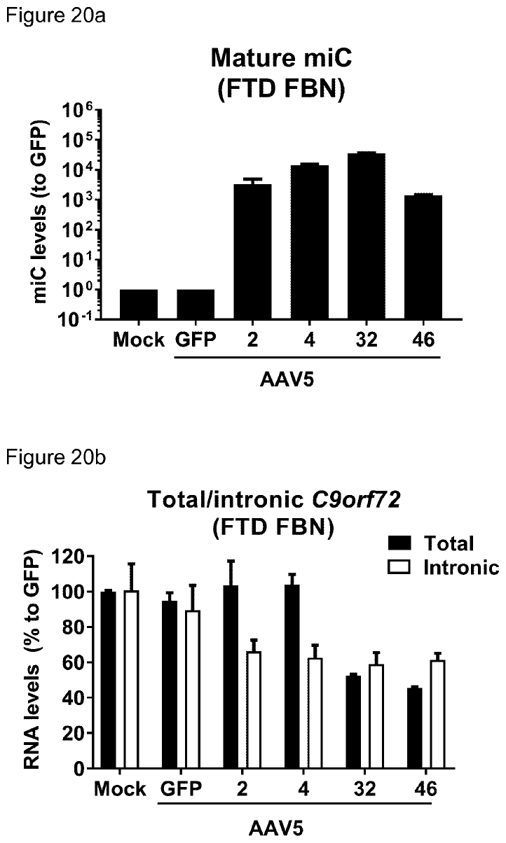

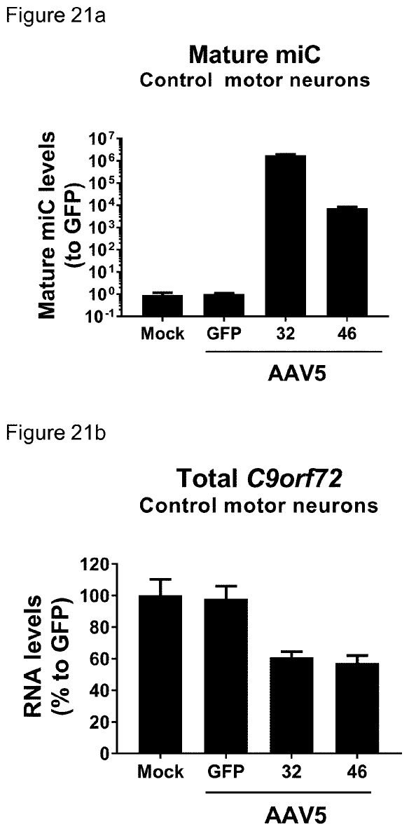

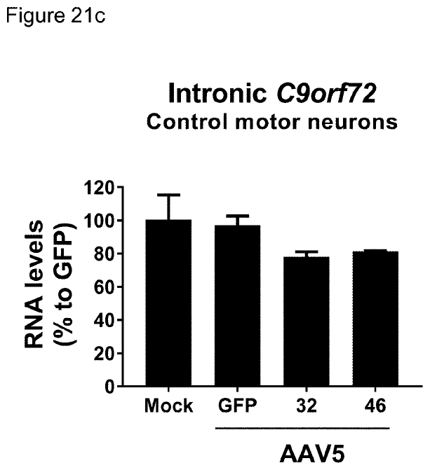

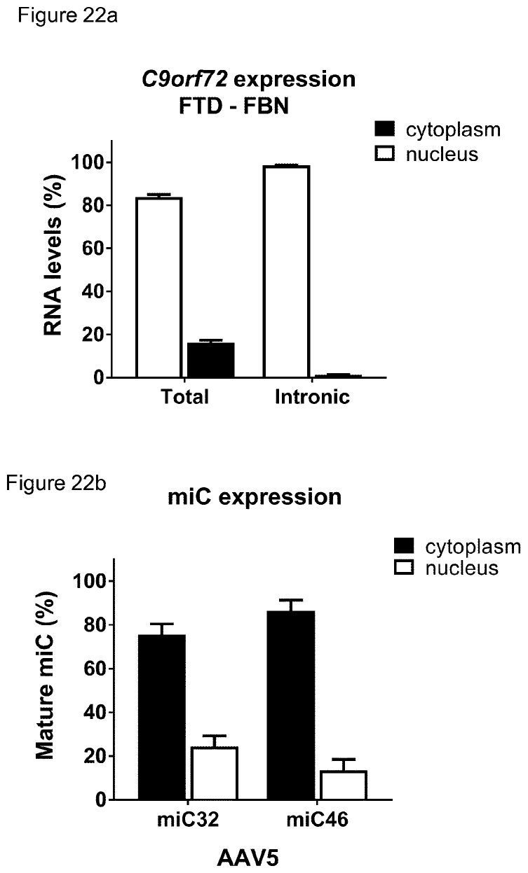

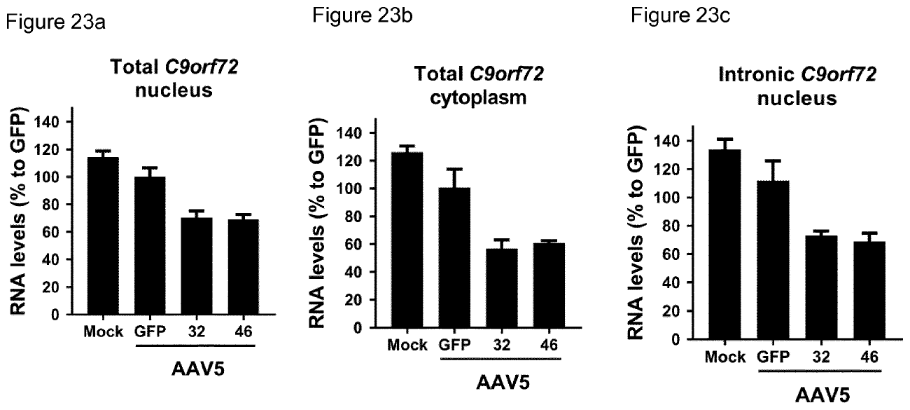

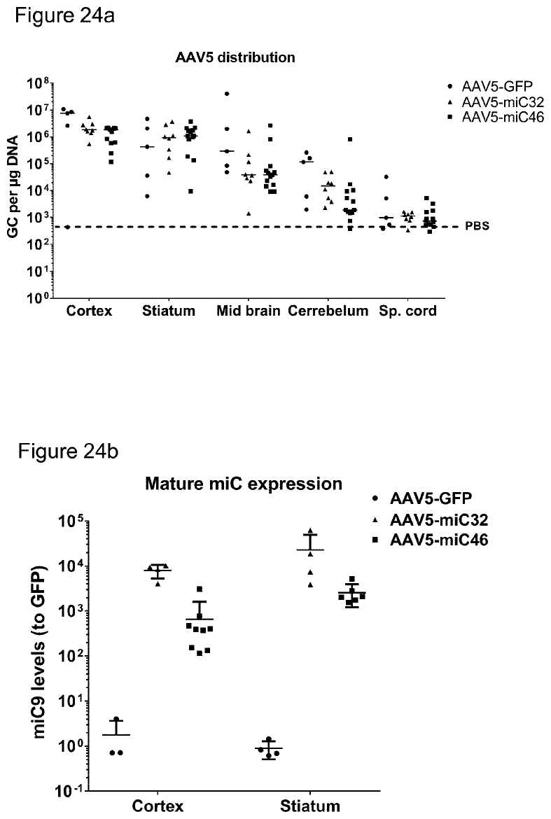

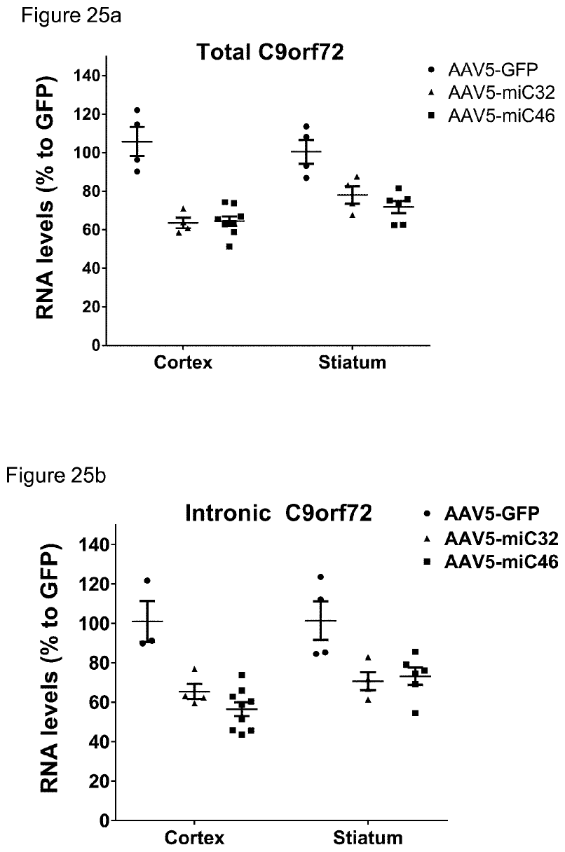

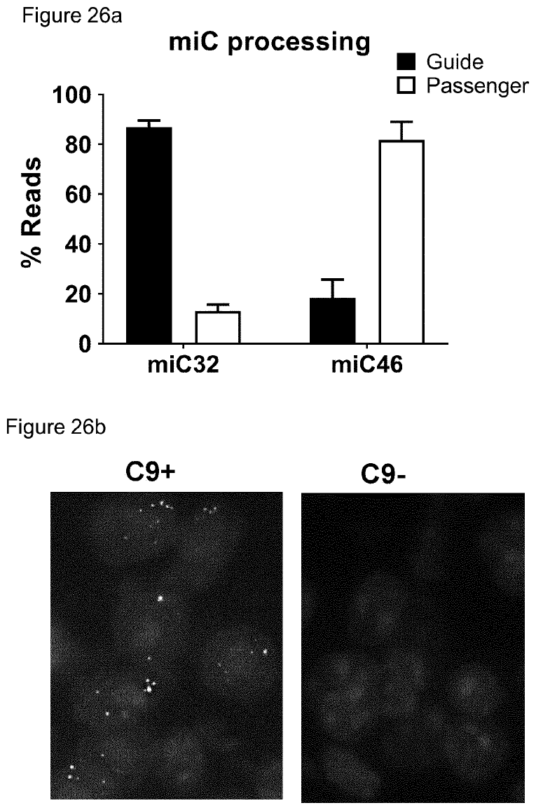

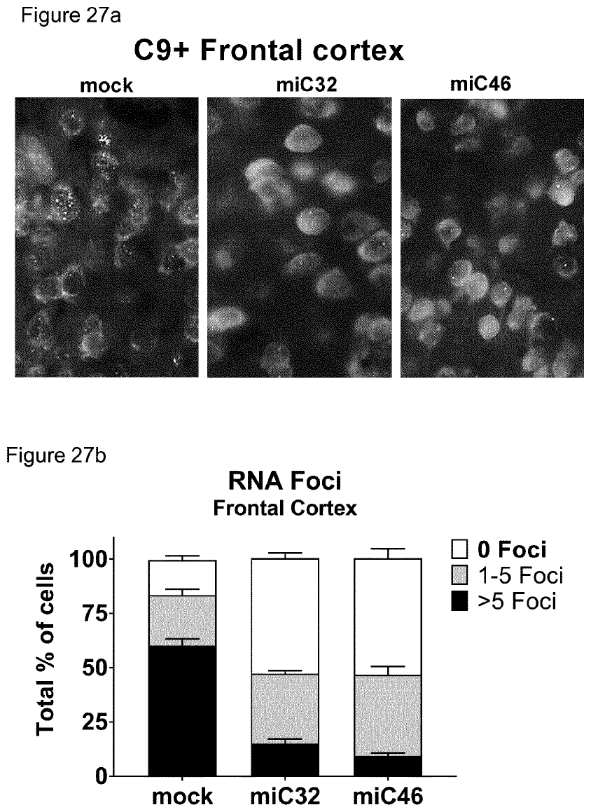

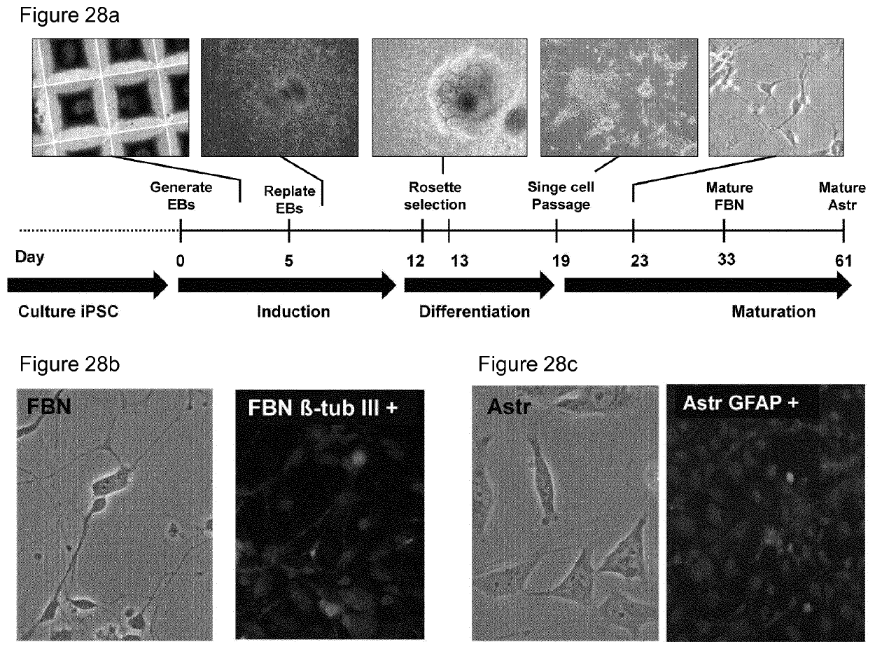

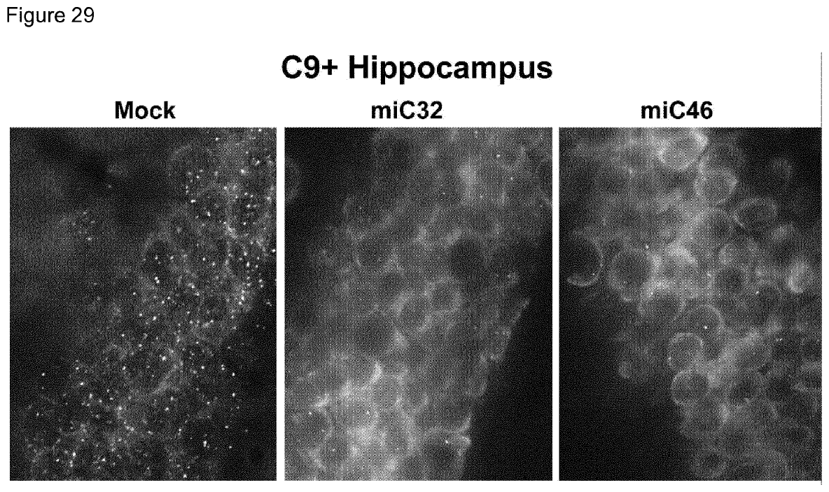

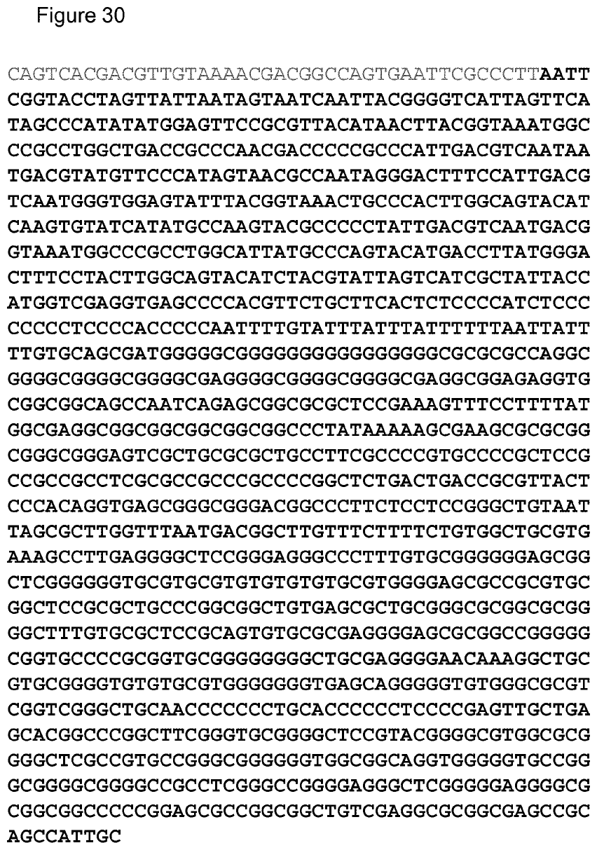

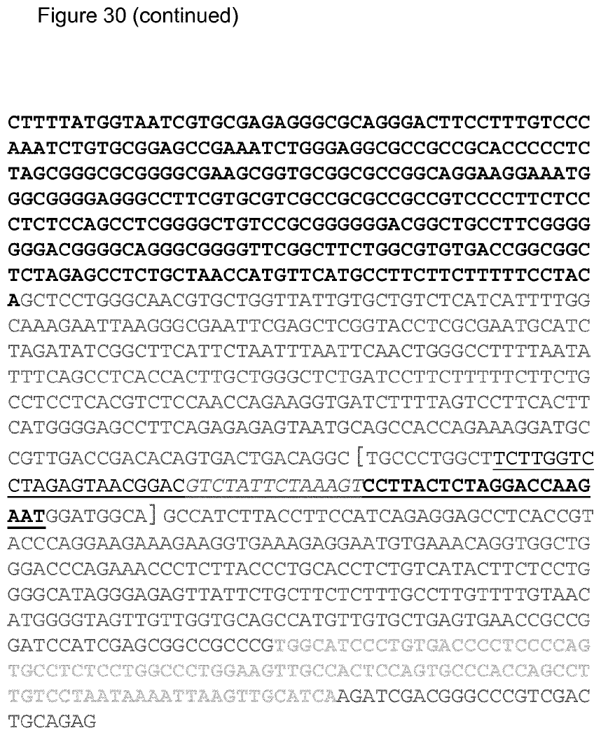

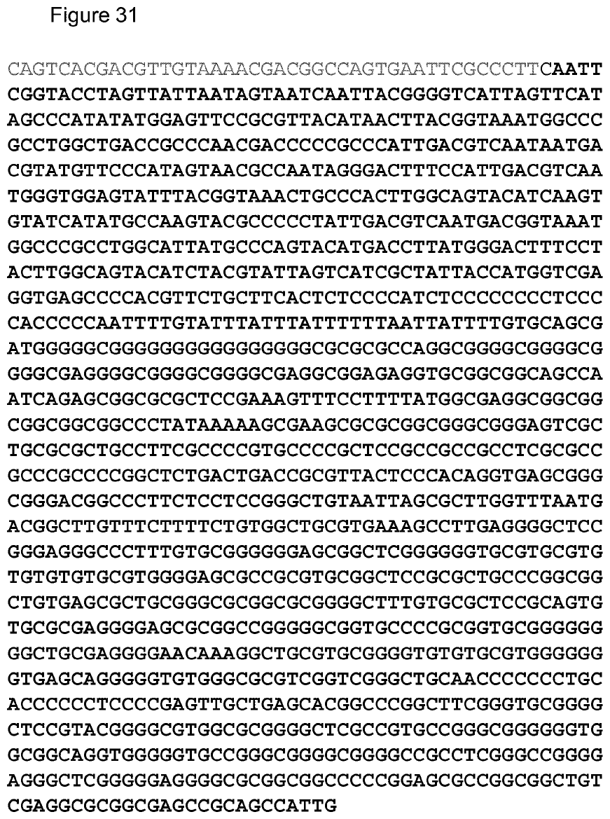

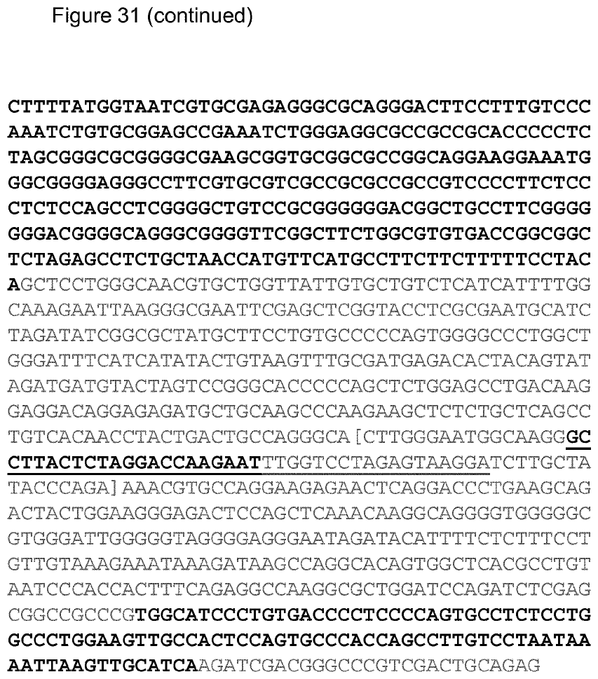

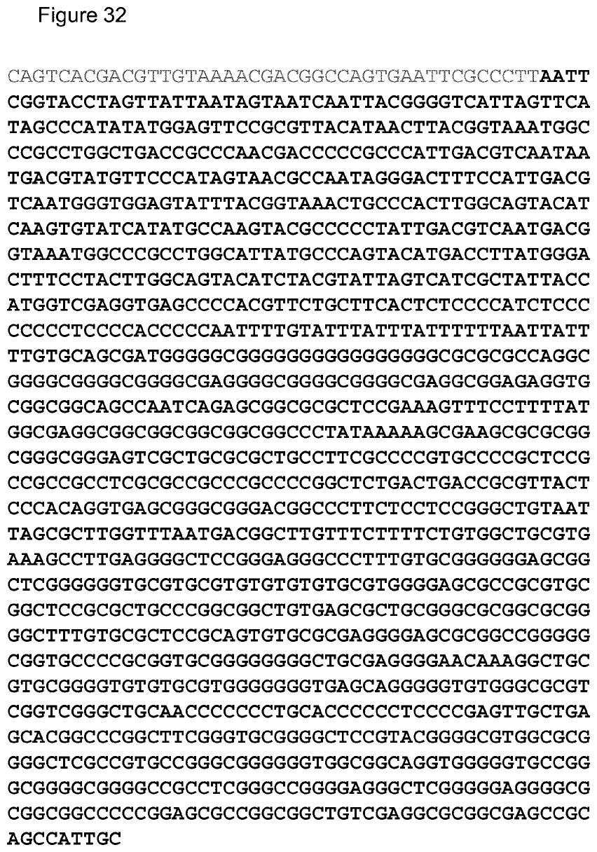

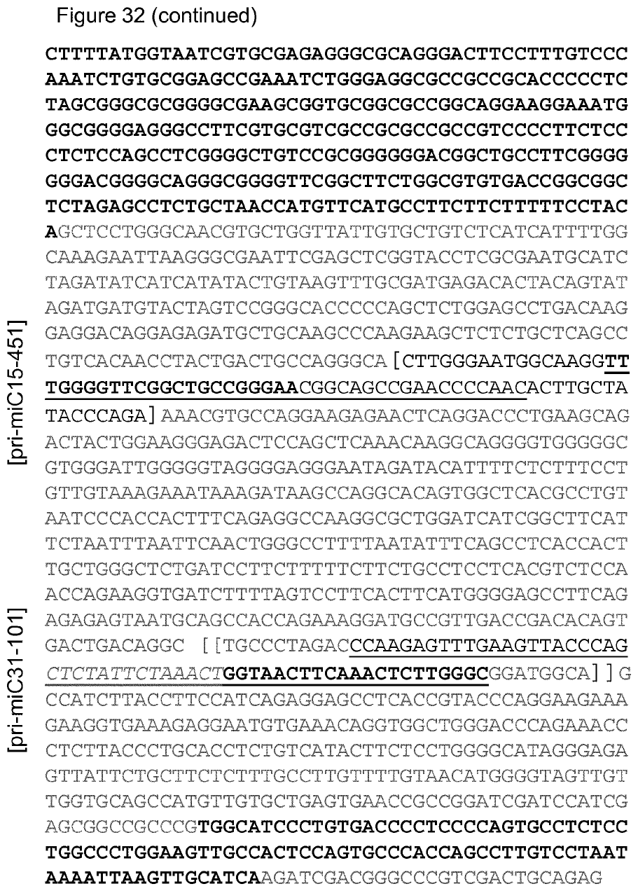

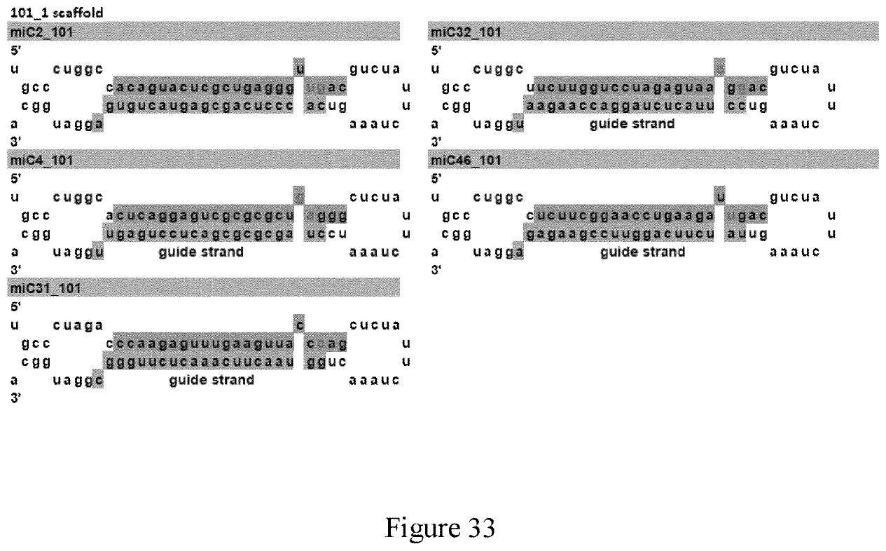

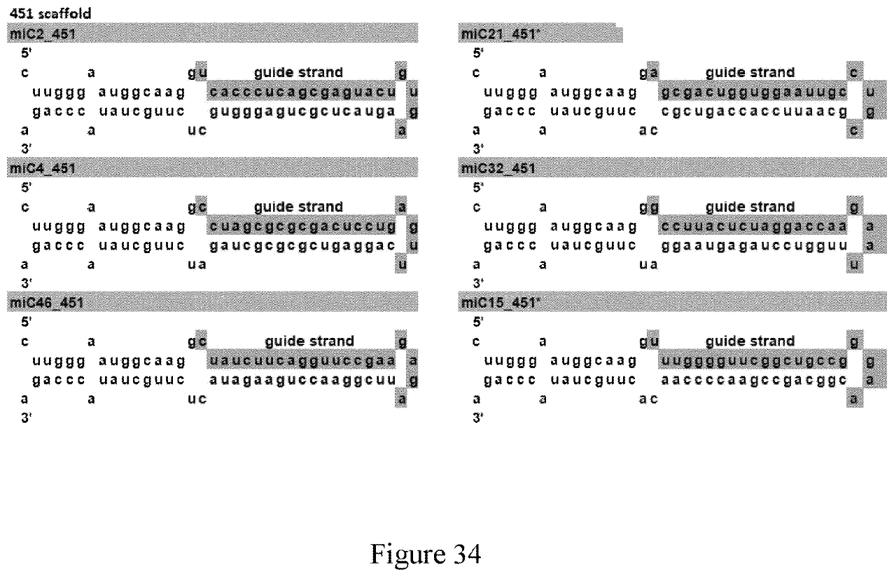

1. An expression cassette encoding a double stranded RNA comprising:
a first RNA sequence, wherein the first RNA sequence has a sequence length of at least 19 nucleotides and is substantially complementarity to SEQ ID NO. 32 and to a target RNA sequence comprised in an RNA encoded by a human C9orf72 gene, and
a second RNA sequence,
wherein the first and second RNA sequence are substantially complementary.

2. The expression cassette according to claim 1, wherein the first and second RNA sequences are comprised in a pre-miRNA scaffold, a pri-miRNA scaffold or a shRNA.

3. The expression cassette according to claim 1, wherein the first and second RNA sequences are comprised in a pre-miRNA scaffold or a pri-miRNA scaffold from miR101 or miR451.

4. The expression cassette according to claim 1, wherein the first RNA sequence is comprised in a guide sequence.

5. The expression cassette according to claim 1, wherein the first RNA sequence and the second RNA sequence, when expressed in a cell, are processed by the cell to produce a guide sequence comprising the first RNA sequence.

6. The expression cassette according to claim claim 1, wherein the first RNA sequence is SEQ ID NO. 86 or SEQ ID NO. 91.

7. The expression cassette according to claim 6, wherein the first RNA sequence and second RNA sequence are selected from the group consisting of the combinations of SEQ ID NOs. 86 and 121, and SEQ ID NOs. 91 and 122 or 157.

8. The expression cassette according to claim 7, wherein the encoded RNA comprises an RNA sequence selected from the group consisting of SEQ ID NOs. 123, 124, 141, 142 and 158.

9. An expression cassette, comprising a combination of a first and second RNA sequence as defined in claim 1 and encoding a second double stranded RNA, the second double stranded RNA comprising a first RNA sequence and a second RNA sequence wherein the first and second RNA sequence of the second double stranded RNA are substantially complementary, and wherein the first RNA sequence of the second double stranded RNA has a sequence length of at least 19 nucleotides and is substantially complementarity to a target RNA sequence comprised in an RNA encoded by a human C9orf72 gene, and wherein said first RNA sequence of said second double stranded RNA is substantially complementary to a target RNA sequence comprised in antisense RNA transcripts encoded by the human C9orf72 gene, wherein said antisense RNA transcript target sequence preferably is SEQ ID NO. 15 or SEQ ID NO. 21.

10. The expression cassette according to claim 1, wherein the expression cassette comprises a PGK promoter, a CMV promoter, a neurospecific promoter or a CBA promoter operably linked to the first RNA sequence and the second RNA sequence.

11. A gene therapy vector, comprising the expression cassette according to claim 1.

12. The gene therapy vector according to claim 11, wherein vector is an AAV vector.

13. The gene therapy vector according to claim 12, wherein the first and second RNA sequences, when expressed in a cell, reduce expression of RNA encoded by a human C9orf72 gene both in the cell nucleus as in the cytoplasm.

14. The gene therapy vector according to claim 12, wherein the first and second RNA sequences, when expressed in a cell, reduce expression of G4C2 foci and/or G2C4 foci.

15. A method of treating ALS and/or FTD, comprising administering to a subject in need thereof a gene therapy vector according according to claim 11.


SEQUENCE LISTING (166 sequences)

SEQ ID NO: 1 - RNA, 21 nt, Artificial Sequence
  Isolated oligonucleotide sequence
cccacccucucuccccacuac

SEQ ID NO: 2 - RNA, 22 nt, Artificial Sequence
  Isolated oligonucleotide sequence
ucacaguacucgcugaggguga

SEQ ID NO: 3 - RNA, 21 nt, Artificial Sequence
  Isolated oligonucleotide sequence
gagggaaacaaccgcagccug

SEQ ID NO: 4 - RNA, 22 nt, Artificial Sequence
  Isolated oligonucleotide sequence
aacucaggagucgcgcgcuagg

SEQ ID NO: 5 - RNA, 22 nt, Artificial Sequence
  Isolated oligonucleotide sequence
agucgcgcgcuaggggccgggg

SEQ ID NO: 6 - RNA, 21 nt, Artificial Sequence
  Isolated oligonucleotide sequence
cuaggggccggggccggggcc

SEQ ID NO: 7 - RNA, 21 nt, Artificial Sequence
  Isolated oligonucleotide sequence
ggggccggggccggggccggg

SEQ ID NO: 8 - RNA, 22 nt, Artificial Sequence
  Isolated oligonucleotide sequence
ucuccccacuacuugcucucac

SEQ ID NO: 9 - RNA, 22 nt, Artificial Sequence
  Isolated oligonucleotide sequence
aguacucgcugagggugaacaa

SEQ ID NO: 10 - RNA, 22 nt, Artificial Sequence
  Isolated oligonucleotide sequence
accugauaaagauuaaccagaa

SEQ ID NO: 11 - RNA, 22 nt, Artificial Sequence
  Isolated oligonucleotide sequence
acaaggagggaaacaaccgcag

SEQ ID NO: 12 - RNA, 22 nt, Artificial Sequence
  Isolated oligonucleotide sequence
ucacucacccacucgccaccgc

SEQ ID NO: 13 - RNA, 22 nt, Artificial Sequence
  Isolated oligonucleotide sequence
aggaugccgccuccucacucac

SEQ ID NO: 14 - RNA, 22 nt, Artificial Sequence
  Isolated oligonucleotide sequence
caaacagccacccgccaggaug

SEQ ID NO: 15 - RNA, 22 nt, Artificial Sequence
  Isolated oligonucleotide sequence
uucccggcagccgaaccccaaa

SEQ ID NO: 16 - RNA, 22 nt, Artificial Sequence
  Isolated oligonucleotide sequence
ccgcuucuacccgcgccucuuc

SEQ ID NO: 17 - RNA, 22 nt, Artificial Sequence
  Isolated oligonucleotide sequence
ugcgucgagcucugaggagagc

SEQ ID NO: 18 - RNA, 22 nt, Artificial Sequence
  Isolated oligonucleotide sequence
ugagagggaaaguaaaaaugcg

SEQ ID NO: 19 - RNA, 22 nt, Artificial Sequence
  Isolated oligonucleotide sequence
cgacacccagcuucggucagag

SEQ ID NO: 20 - RNA, 22 nt, Artificial Sequence
  Isolated oligonucleotide sequence
agucgcuagaggcgaaagcccg

SEQ ID NO: 21 - RNA, 22 nt, Artificial Sequence
  Isolated oligonucleotide sequence
gcaggcaauuccaccagucgcu

SEQ ID NO: 22 - RNA, 22 nt, Artificial Sequence
  Isolated oligonucleotide sequence
ucgggguucgcuaggaacccga

SEQ ID NO: 23 - RNA, 22 nt, Artificial Sequence
  Isolated oligonucleotide sequence
aggagaucaugcgggaugagau

SEQ ID NO: 24 - RNA, 22 nt, Artificial Sequence
  Isolated oligonucleotide sequence
uggagacgccugcacaauuuca

SEQ ID NO: 25 - RNA, 22 nt, Artificial Sequence
  Isolated oligonucleotide sequence
aguggugaugacuugcauauga

SEQ ID NO: 26 - RNA, 22 nt, Artificial Sequence
  Isolated oligonucleotide sequence
augcaagucggugugcucccca

SEQ ID NO: 27 - RNA, 22 nt, Artificial Sequence
  Isolated oligonucleotide sequence
ugugggacaugaccugguugcu

SEQ ID NO: 28 - RNA, 22 nt, Artificial Sequence
  Isolated oligonucleotide sequence
cagcuccgagaugacacagacu

SEQ ID NO: 29 - RNA, 22 nt, Artificial Sequence
  Isolated oligonucleotide sequence
auugugacuugggcaucacuug

SEQ ID NO: 30 - RNA, 21 nt, Artificial Sequence
  Isolated oligonucleotide sequence
uugacugaugguaaucaguug

SEQ ID NO: 31 - RNA, 21 nt, Artificial Sequence
  Isolated oligonucleotide sequence
gcccaagaguuugaaguuacc

SEQ ID NO: 32 - RNA, 22 nt, Artificial Sequence
  Isolated oligonucleotide sequence
auucuugguccuagaguaaggc

SEQ ID NO: 33 - RNA, 21 nt, Artificial Sequence
  Isolated oligonucleotide sequence
uucuugguccuagaguaaggc

SEQ ID NO: 34 - RNA, 22 nt, Artificial Sequence
  Isolated oligonucleotide sequence
cuucucagugauggagaaauaa

SEQ ID NO: 35 - RNA, 22 nt, Artificial Sequence
  Isolated oligonucleotide sequence
ccaaccacacucuaaauggaga

SEQ ID NO: 36 - RNA, 22 nt, Artificial Sequence
  Isolated oligonucleotide sequence
ggaagaauauggaugcauaaga

SEQ ID NO: 37 - RNA, 22 nt, Artificial Sequence
  Isolated oligonucleotide sequence
agcuguugccaagacagagauu

SEQ ID NO: 38 - RNA, 22 nt, Artificial Sequence
  Isolated oligonucleotide sequence
uguugccaagacagagauugcu

SEQ ID NO: 39 - RNA, 22 nt, Artificial Sequence
  Isolated oligonucleotide sequence
ugccaagacagagauugcuuua

SEQ ID NO: 40 - RNA, 22 nt, Artificial Sequence
  Isolated oligonucleotide sequence
caagacagagauugcuuuaagu

SEQ ID NO: 41 - RNA, 22 nt, Artificial Sequence
  Isolated oligonucleotide sequence
agacagagauugcuuuaagugg

SEQ ID NO: 42 - RNA, 22 nt, Artificial Sequence
  Isolated oligonucleotide sequence
uagaguaaggcacauuugggcu

SEQ ID NO: 43 - RNA, 22 nt, Artificial Sequence
  Isolated oligonucleotide sequence
gagaaauaacuuuucuugccaa

SEQ ID NO: 44 - RNA, 22 nt, Artificial Sequence
  Isolated oligonucleotide sequence
agugugguuggcaagaaaaguu

SEQ ID NO: 45 - RNA, 22 nt, Artificial Sequence
  Isolated oligonucleotide sequence
ugccaaccacacucuaaaugga

SEQ ID NO: 46 - RNA, 22 nt, Artificial Sequence
  Isolated oligonucleotide sequence
ucucuucggaaccugaagauag

SEQ ID NO: 47 - RNA, 21 nt, Artificial Sequence
  Isolated oligonucleotide sequence
cuugauuuaacagcagagggc

SEQ ID NO: 48 - RNA, 21 nt, Artificial Sequence
  Isolated oligonucleotide sequence
ucuuaacauaauaauggcucu

SEQ ID NO: 49 - RNA, 22 nt, Artificial Sequence
  Isolated oligonucleotide sequence
gagcuugaacauaggaugagcu

SEQ ID NO: 50 - RNA, 22 nt, Artificial Sequence
  Isolated oligonucleotide sequence
aauacuaccuuguaguguccca

SEQ ID NO: 51 - RNA, 21 nt, Artificial Sequence
  Isolated oligonucleotide sequence
guaguggggagagaggguggg

SEQ ID NO: 52 - RNA, 21 nt, Artificial Sequence
  Isolated oligonucleotide sequence
cacccucagcgaguacuguga

SEQ ID NO: 53 - RNA, 21 nt, Artificial Sequence
  Isolated oligonucleotide sequence
caggcugcgguuguuucccuc

SEQ ID NO: 54 - RNA, 21 nt, Artificial Sequence
  Isolated oligonucleotide sequence
cuagcgcgcgacuccugaguu

SEQ ID NO: 55 - RNA, 21 nt, Artificial Sequence
  Isolated oligonucleotide sequence
cccggccccuagcgcgcgacu

SEQ ID NO: 56 - RNA, 21 nt, Artificial Sequence
  Isolated oligonucleotide sequence
ggccccggccccggccccuag

SEQ ID NO: 57 - RNA, 21 nt, Artificial Sequence
  Isolated oligonucleotide sequence
cccggccccggccccggcccc

SEQ ID NO: 58 - RNA, 22 nt, Artificial Sequence
  Isolated oligonucleotide sequence
ucacccucagcgaguacuguga

SEQ ID NO: 59 - RNA, 22 nt, Artificial Sequence
  Isolated oligonucleotide sequence
ccuagcgcgcgacuccugaguu

SEQ ID NO: 60 - RNA, 22 nt, Artificial Sequence
  Isolated oligonucleotide sequence
cccggccccuagcgcgcgacuc

SEQ ID NO: 61 - RNA, 22 nt, Artificial Sequence
  Isolated oligonucleotide sequence
gugagagcaaguaguggggaga

SEQ ID NO: 62 - RNA, 22 nt, Artificial Sequence
  Isolated oligonucleotide sequence
uuguucacccucagcgaguacu

SEQ ID NO: 63 - RNA, 22 nt, Artificial Sequence
  Isolated oligonucleotide sequence
uucugguuaaucuuuaucaggu

SEQ ID NO: 64 - RNA, 22 nt, Artificial Sequence
  Isolated oligonucleotide sequence
cugcgguuguuucccuccuugu

SEQ ID NO: 65 - RNA, 22 nt, Artificial Sequence
  Isolated oligonucleotide sequence
gcgguggcgagugggugaguga

SEQ ID NO: 66 - RNA, 22 nt, Artificial Sequence
  Isolated oligonucleotide sequence
gugagugaggaggcggcauccu

SEQ ID NO: 67 - RNA, 22 nt, Artificial Sequence
  Isolated oligonucleotide sequence
cauccuggcggguggcuguuug

SEQ ID NO: 68 - RNA, 22 nt, Artificial Sequence
  Isolated oligonucleotide sequence
uuugggguucggcugccgggaa

SEQ ID NO: 69 - RNA, 22 nt, Artificial Sequence
  Isolated oligonucleotide sequence
gaagaggcgcggguagaagcgg

SEQ ID NO: 70 - RNA, 22 nt, Artificial Sequence
  Isolated oligonucleotide sequence
gcucuccucagagcucgacgca

SEQ ID NO: 71 - RNA, 22 nt, Artificial Sequence
  Isolated oligonucleotide sequence
cgcauuuuuacuuucccucuca

SEQ ID NO: 72 - RNA, 22 nt, Artificial Sequence
  Isolated oligonucleotide sequence
cucugaccgaagcugggugucg

SEQ ID NO: 73 - RNA, 22 nt, Artificial Sequence
  Isolated oligonucleotide sequence
cgggcuuucgccucuagcgacu

SEQ ID NO: 74 - RNA, 22 nt, Artificial Sequence
  Isolated oligonucleotide sequence
agcgacugguggaauugccugc

SEQ ID NO: 75 - RNA, 22 nt, Artificial Sequence
  Isolated oligonucleotide sequence
ucggguuccuagcgaaccccga

SEQ ID NO: 76 - RNA, 22 nt, Artificial Sequence
  Isolated oligonucleotide sequence
aucucaucccgcaugaucuccu

SEQ ID NO: 77 - RNA, 22 nt, Artificial Sequence
  Isolated oligonucleotide sequence
ugaaauugugcaggcgucucca

SEQ ID NO: 78 - RNA, 22 nt, Artificial Sequence
  Isolated oligonucleotide sequence
ucauaugcaagucaucaccacu

SEQ ID NO: 79 - RNA, 22 nt, Artificial Sequence
  Isolated oligonucleotide sequence
uggggagcacaccgacuugcau

SEQ ID NO: 80 - RNA, 22 nt, Artificial Sequence
  Isolated oligonucleotide sequence
agcaaccaggucaugucccaca

SEQ ID NO: 81 - RNA, 22 nt, Artificial Sequence
  Isolated oligonucleotide sequence
agucugugucaucucggagcug

SEQ ID NO: 82 - RNA, 21 nt, Artificial Sequence
  Isolated oligonucleotide sequence
aagugaugcccaagucacaau

SEQ ID NO: 83 - RNA, 22 nt, Artificial Sequence
  Isolated oligonucleotide sequence
caagugaugcccaagucacaau

SEQ ID NO: 84 - RNA, 21 nt, Artificial Sequence
  Isolated oligonucleotide sequence
caacugauuaccaucagucaa

SEQ ID NO: 85 - RNA, 21 nt, Artificial Sequence
  Isolated oligonucleotide sequence
gguaacuucaaacucuugggc

SEQ ID NO: 86 - RNA, 21 nt, Artificial Sequence
  Isolated oligonucleotide sequence
ccuuacucuaggaccaagaau

SEQ ID NO: 87 - RNA, 21 nt, Artificial Sequence
  Isolated oligonucleotide sequence
gccuuacucuaggaccaagaa

SEQ ID NO: 88 - RNA, 21 nt, Artificial Sequence
  Isolated oligonucleotide sequence
uauuucuccaucacugagaag

SEQ ID NO: 89 - RNA, 21 nt, Artificial Sequence
  Isolated oligonucleotide sequence
cuccauuuagagugugguugg

SEQ ID NO: 90 - RNA, 21 nt, Artificial Sequence
  Isolated oligonucleotide sequence
cuuaugcauccauauucuucc

SEQ ID NO: 91 - RNA, 22 nt, Artificial Sequence
  Isolated oligonucleotide sequence
gccuuacucuaggaccaagaau

SEQ ID NO: 92 - RNA, 22 nt, Artificial Sequence
  Isolated oligonucleotide sequence
uuauuucuccaucacugagaag

SEQ ID NO: 93 - RNA, 22 nt, Artificial Sequence
  Isolated oligonucleotide sequence
uucuccauuuagagugugguug

SEQ ID NO: 94 - RNA, 22 nt, Artificial Sequence
  Isolated oligonucleotide sequence
uuaugcauccauauucuuccuu

SEQ ID NO: 95 - RNA, 22 nt, Artificial Sequence
  Isolated oligonucleotide sequence
aaucucugucuuggcaacagcu

SEQ ID NO: 96 - RNA, 22 nt, Artificial Sequence
  Isolated oligonucleotide sequence
agcaaucucugucuuggcaaca

SEQ ID NO: 97 - RNA, 22 nt, Artificial Sequence
  Isolated oligonucleotide sequence
uaaagcaaucucugucuuggca

SEQ ID NO: 98 - RNA, 22 nt, Artificial Sequence
  Isolated oligonucleotide sequence
acuuaaagcaaucucugucuug

SEQ ID NO: 99 - RNA, 22 nt, Artificial Sequence
  Isolated oligonucleotide sequence
ccacuuaaagcaaucucugucu

SEQ ID NO: 100 - RNA, 22 nt, Artificial Sequence
  Isolated oligonucleotide sequence
agcccaaaugugccuuacucua

SEQ ID NO: 101 - RNA, 22 nt, Artificial Sequence
  Isolated oligonucleotide sequence
uuggcaagaaaaguuauuucuc

SEQ ID NO: 102 - RNA, 22 nt, Artificial Sequence
  Isolated oligonucleotide sequence
aacuuuucuugccaaccacacu

SEQ ID NO: 103 - RNA, 22 nt, Artificial Sequence
  Isolated oligonucleotide sequence
uccauuuagagugugguuggca

SEQ ID NO: 104 - RNA, 21 nt, Artificial Sequence
  Isolated oligonucleotide sequence
uaucuucagguuccgaagaga

SEQ ID NO: 105 - RNA, 21 nt, Artificial Sequence
  Isolated oligonucleotide sequence
gcccucugcuguuaaaucaag

SEQ ID NO: 106 - RNA, 21 nt, Artificial Sequence
  Isolated oligonucleotide sequence
agagccauuauuauguuaaga

SEQ ID NO: 107 - RNA, 21 nt, Artificial Sequence
  Isolated oligonucleotide sequence
gcucauccuauguucaagcuc

SEQ ID NO: 108 - RNA, 21 nt, Artificial Sequence
  Isolated oligonucleotide sequence
gggacacuacaagguaguauu

SEQ ID NO: 109 - RNA, 22 nt, Artificial Sequence
  Isolated oligonucleotide sequence
cuaucuucagguuccgaagaga

SEQ ID NO: 110 - RNA, 22 nt, Artificial Sequence
  Isolated oligonucleotide sequence
agcucauccuauguucaagcuc

SEQ ID NO: 111 - RNA, 22 nt, Artificial Sequence
  Isolated oligonucleotide sequence
ugggacacuacaagguaguauu

SEQ ID NO: 112 - RNA, 16 nt, Artificial Sequence
  Isolated oligonucleotide sequence
cuugggaauggcaagg

SEQ ID NO: 113 - RNA, 16 nt, Artificial Sequence
  Isolated oligonucleotide sequence
mwcuugcuaucccaga

SEQ ID NO: 114 - RNA, 10 nt, Artificial Sequence
  Isolated oligonucleotide sequence
  misc_feature (9)..(10): n is a, c, g, or u
ugcccyggnn

SEQ ID NO: 115 - RNA, 14 nt, Artificial Sequence
  Isolated oligonucleotide sequence
  misc_feature (13)..(14): n is a, c, g, or u
sucuauucuaaann

SEQ ID NO: 116 - RNA, 8 nt, Artificial Sequence
  Isolated oligonucleotide sequence
dsauggca

SEQ ID NO: 117 - RNA, 18 nt, Artificial Sequence
  Isolated oligonucleotide sequence
cggcagccgaaccccaac

SEQ ID NO: 118 - RNA, 18 nt, Artificial Sequence
  Isolated oligonucleotide sequence
gcaauuccaccagucgcc

SEQ ID NO: 119 - RNA, 40 nt, Artificial Sequence
  Isolated oligonucleotide sequence
uuugggguucggcugccgggaacggcagccgaaccccaac

SEQ ID NO: 120 - RNA, 40 nt, Artificial Sequence
  Isolated oligonucleotide sequence
agcgacugguggaauugccugcgcaauuccaccagucgcc

SEQ ID NO: 121 - RNA, 22 nt, Artificial Sequence
  Isolated oligonucleotide sequence
ucuugguccuagaguaacggac

SEQ ID NO: 122 - RNA, 18 nt, Artificial Sequence
  Isolated oligonucleotide sequence
uugguccuagaguaagga

SEQ ID NO: 123 - RNA, 57 nt, Artificial Sequence
  Isolated oligonucleotide sequence
ucuugguccuagaguaacggacgucuauucuaaaguccuuacucuaggaccaagaau

SEQ ID NO: 124 - RNA, 40 nt, Artificial Sequence
  Isolated oligonucleotide sequence
gccuuacucuaggaccaagaauuugguccuagaguaagga

SEQ ID NO: 125 - RNA, 22 nt, Artificial Sequence
  Isolated oligonucleotide sequence
acaguacucgcugaggguugac

SEQ ID NO: 126 - RNA, 18 nt, Artificial Sequence
  Isolated oligonucleotide sequence
aguacucgcugagggugc

SEQ ID NO: 127 - RNA, 22 nt, Artificial Sequence
  Isolated oligonucleotide sequence
cucaggagucgcgcgcugaggg

SEQ ID NO: 128 - RNA, 18 nt, Artificial Sequence
  Isolated oligonucleotide sequence
caggagucgcgcgcuaga

SEQ ID NO: 129 - RNA, 22 nt, Artificial Sequence
  Isolated oligonucleotide sequence
ccaagaguuugaaguuacccag

SEQ ID NO: 130 - RNA, 57 nt, Artificial Sequence
  Isolated oligonucleotide sequence
acaguacucgcugaggguugacgucuauucuaaagucacccucagcgaguacuguga

SEQ ID NO: 131 - RNA, 40 nt, Artificial Sequence
  Isolated oligonucleotide sequence
ucacccucagcgaguacugugaaguacucgcugagggugc

SEQ ID NO: 132 - RNA, 57 nt, Artificial Sequence
  Isolated oligonucleotide sequence
cucaggagucgcgcgcugagggcucuauucuaaauccuagcgcgcgacuccugaguu

SEQ ID NO: 133 - RNA, 40 nt, Artificial Sequence
  Isolated oligonucleotide sequence
ccuagcgcgcgacuccugaguucaggagucgcgcgcuaga

SEQ ID NO: 134 - RNA, 57 nt, Artificial Sequence
  Isolated oligonucleotide sequence
ccaagaguuugaaguuacccagcucuauucuaaacugguaacuucaaacucuugggc

SEQ ID NO: 135 - RNA, 22 nt, Artificial Sequence
  Isolated oligonucleotide sequence
ucuucggaaccugaagauugac

SEQ ID NO: 136 - RNA, 18 nt, Artificial Sequence
  Isolated oligonucleotide sequence
uucggaaccugaagauac

SEQ ID NO: 137 - RNA, 57 nt, Artificial Sequence
  Isolated oligonucleotide sequence
ucuucggaaccugaagauugacgucuauucuaaaguuaucuucagguuccgaagaga

SEQ ID NO: 138 - RNA, 40 nt, Artificial Sequence
  Isolated oligonucleotide sequence
cuaucuucagguuccgaagagauucggaaccugaagauac

SEQ ID NO: 139 - RNA, 72 nt, Artificial Sequence
  Isolated oligonucleotide sequence
cuugggaauggcaagguuugggguucggcugccgggaacggcagccgaaccccaacacuugcuauacccaga

SEQ ID NO: 140 - RNA, 72 nt, Artificial Sequence
  Isolated oligonucleotide sequence
cuugggaauggcaaggagcgacugguggaauugccugcgcaauuccaccagucgccacuugcuauacccaga

SEQ ID NO: 141 - RNA, 75 nt, Artificial Sequence
  Isolated oligonucleotide sequence
ugcccuggcuucuugguccuagaguaacggacgucuauucuaaaguccuuacucuaggaccaagaauggauggca

SEQ ID NO: 142 - RNA, 72 nt, Artificial Sequence
  Isolated oligonucleotide sequence
cuugggaauggcaagggccuuacucuaggaccaagaauuugguccuagaguaaggaucuugcuauacccaga

SEQ ID NO: 143 - RNA, 75 nt, Artificial Sequence
  Isolated oligonucleotide sequence
ugcccuggccacaguacucgcugaggguugacgucuauucuaaagucacccucagcgaguacugugaggauggca

SEQ ID NO: 144 - RNA, 72 nt, Artificial Sequence
  Isolated oligonucleotide sequence
cuugggaauggcaaggucacccucagcgaguacugugaaguacucgcugagggugcucuugcuauacccaga

SEQ ID NO: 145 - RNA, 75 nt, Artificial Sequence
  Isolated oligonucleotide sequence
ugcccuggcacucaggagucgcgcgcugagggcucuauucuaaauccuagcgcgcgacuccugaguuggauggca

SEQ ID NO: 146 - RNA, 72 nt, Artificial Sequence
  Isolated oligonucleotide sequence
cuugggaauggcaaggccuagcgcgcgacuccugaguucaggagucgcgcgcuagaucuugcuauacccaga

SEQ ID NO: 147 - RNA, 75 nt, Artificial Sequence
  Isolated oligonucleotide sequence
ugcccuagacccaagaguuugaaguuacccagcucuauucuaaacugguaacuucaaacucuugggcggauggca

SEQ ID NO: 148 - RNA, 75 nt, Artificial Sequence
  Isolated oligonucleotide sequence
ugcccuggccucuucggaaccugaagauugacgucuauucuaaaguuaucuucagguuccgaagagaggauggca

SEQ ID NO: 149 - RNA, 72 nt, Artificial Sequence
  Isolated oligonucleotide sequence
cuugggaauggcaaggcuaucuucagguuccgaagagauucggaaccugaagauacucuugcuauacccaga

SEQ ID NO: 150 - DNA, 2452 bp, Artificial Sequence
  expression cassette encoding a miR101 scaffold
cagtcacgacgttgtaaaacgacggccagtgaattcgcccttaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaagggcgaattcgagctcggtacctcgcgaatgcatctagatatcggcttcattctaatttaattcaactgggccttttaatatttcagcctcaccacttgctgggctctgatccttctttttcttctgcctcctcacgtctccaaccagaaggtgatcttttagtccttcacttcatggggagccttcagagagagtaatgcagccaccagaaaggatgccgttgaccgacacagtgactgacaggctgccctggcttcttggtcctagagtaacggacgtctattctaaagtccttactctaggaccaagaatggatggcagccatcttaccttccatcagaggagcctcaccgtacccaggaagaaagaaggtgaaagaggaatgtgaaacaggtggctgggacccagaaaccctcttaccctgcacctctgtcatacttctcctggggcatagggagagttattctgcttctctttgccttgttttgtaacatggggtagttgttggtgcagccatgttgtgctgagtgaaccgccggatccatcgagcggccgcccgtggcatccctgtgacccctccccagtgcctctcctggccctggaagttgccactccagtgcccaccagccttgtcctaataaaattaagttgcatcaagatcgacgggcccgtcgactgcagag

SEQ ID NO: 151 - DNA, 2441 bp, Artificial Sequence
  expression construct encoding a miR451 scaffold
cagtcacgacgttgtaaaacgacggccagtgaattcgcccttcaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgcttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaagggcgaattcgagctcggtacctcgcgaatgcatctagatatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggccttactctaggaccaagaatttggtcctagagtaaggatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatccagatctcgagcggccgcccgtggcatccctgtgacccctccccagtgcctctcctggccctggaagttgccactccagtgcccaccagccttgtcctaataaaattaagttgcatcaagatcgacgggcccgtcgactgcagag

SEQ ID NO: 152 - DNA, 2898 bp, Artificial Sequence
  expression construct encoding a single RNA transcript with a miR451 scaffold
cagtcacgacgttgtaaaacgacggccagtgaattcgcccttaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaagggcgaattcgagctcggtacctcgcgaatgcatctagatatcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtttggggttcggctgccgggaacggcagccgaaccccaacacttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcatcggcttcattctaatttaattcaactgggccttttaatatttcagcctcaccacttgctgggctctgatccttctttttcttctgcctcctcacgtctccaaccagaaggtgatcttttagtccttcacttcatggggagccttcagagagagtaatgcagccaccagaaaggatgccgttgaccgacacagtgactgacaggctgccctagacccaagagtttgaagttacccagctctattctaaactggtaacttcaaactcttgggcggatggcagccatcttaccttccatcagaggagcctcaccgtacccaggaagaaagaaggtgaaagaggaatgtgaaacaggtggctgggacccagaaaccctcttaccctgcacctctgtcatacttctcctggggcatagggagagttattctgcttctctttgccttgttttgtaacatggggtagttgttggtgcagccatgttgtgctgagtgaaccgccggatcgatccatcgagcggccgcccgtggcatccctgtgacccctccccagtgcctctcctggccctggaagttgccactccagtgcccaccagccttgtcctaataaaattaagttgcatcaagatcgacgggcccgtcgactgcagag

SEQ ID NO: 153 - RNA, 17 nt, Artificial Sequence
  Isolated oligonucleotide sequence
cggcagccgaaccccaa

SEQ ID NO: 154 - RNA, 17 nt, Artificial Sequence
  Isolated oligonucleotide sequence
gcaauuccaccagucgc

SEQ ID NO: 155 - RNA, 39 nt, Artificial Sequence
  Isolated oligonucleotide sequence
uuugggguucggcugccgggaacggcagccgaaccccaa

SEQ ID NO: 156 - RNA, 39 nt, Artificial Sequence
  Isolated oligonucleotide sequence
agcgacugguggaauugccugcgcaauuccaccagucgc

SEQ ID NO: 157 - RNA, 17 nt, Artificial Sequence
  Isolated oligonucleotide sequence
uugguccuagaguaagg

SEQ ID NO: 158 - RNA, 39 nt, Artificial Sequence
  Isolated oligonucleotide sequence
gccuuacucuaggaccaagaauuugguccuagaguaagg

SEQ ID NO: 159 - RNA, 17 nt, Artificial Sequence
  Isolated oligonucleotide sequence
aguacucgcugagggug

SEQ ID NO: 160 - RNA, 17 nt, Artificial Sequence
  Isolated oligonucleotide sequence
caggagucgcgcgcuag

SEQ ID NO: 161 - RNA, 39 nt, Artificial Sequence
  Isolated oligonucleotide sequence
ucacccucagcgaguacugugaaguacucgcugagggug

SEQ ID NO: 162 - RNA, 39 nt, Artificial Sequence
  Isolated oligonucleotide sequence
ccuagcgcgcgacuccugaguucaggagucgcgcgcuag

SEQ ID NO: 163 - RNA, 17 nt, Artificial Sequence
  Isolated oligonucleotide sequence
uucggaaccugaagaua

SEQ ID NO: 164 - RNA, 39 nt, Artificial Sequence
  Isolated oligonucleotide sequence
cuaucuucagguuccgaagagauucggaaccugaagaua

SEQ ID NO: 165 - RNA, 75 nt, Artificial Sequence
  Pre-miC_101
  misc_feature (10)..(30): n is a, c, g, or u
  misc_feature (47)..(67): n is a, c, g, or u
ugcccggggnnnnnnnnnnnnnnnnnnnnnuggucuauucuaaacgnnnnnnnnnnnnnnnnnnnnnagauggca

SEQ ID NO: 166 - RNA, 72 nt, Artificial Sequence
  Pre-miC_451
  misc_feature (17)..(56): n is a, c, g, or u
cuugggaauggcaaggnnnnnnnnnnnnnnnnnnnnnnnnnnnnnnnnnnnnnnnnacuugcuauacccaga